SEQUENCE LISTING (619 sequences)

SEQ ID NO: 1 - DNA, 2074 bp, Homo sapiens
tagggggtggccctgaactggggcctggccctggctggcctctcccgccgcctcactgggggacaggtccagcctgtggtgtccacaatgccccaggcctctgagcaccgcctgggccgtacccgagagccacctgttaatatccagccccgagtgggatccaagctaccatttgcccccagggcccgcagcaaggagcgcagaaacccagcctctgggccaaaccccatgttacgacctctgcctccccggccaggtctgcctgatgaacggctcaagaaactggagctgggacggggacggacctcaggccctcgtcccagaggcccccttcgagcagatcatggggttcccctgcctggctcaccacccccaacagtggctttgcctctcccatctcggaccaacttagcccgttccaagtctgtgagcagtggggacttgcgtccaatggggattgccttgggagggcaccgtggcaccggagagcttggggctgcactgagccgcttggccctccggcctgagccacccactttgagacgtagcacttctctccgccgcctagggggctttcctggaccccctaccctgttcagcatacggacagagccccctgcttcccatggctccttccacatgatatccgcccggtcctctgagcctttctactctgatgacaagatggctcatcacacactccttctgggctctggtcatgttggccttcgaaacctgggaaacacgtgcttcctgaatgctgtgctgcagtgtctgagcagcactcgacctcttcgggacttctgtctgagaagggacttccggcaagaggtgcctggaggaggccgagcccaagagctcactgaagcctttgcagatgtgattggtgccctctggcaccctgactcctgcgaagctgtgaatcctactcgattccgagctgtcttccagaaatatgttccctccttctctggatacagccagcaggatgcccaagagttcctgaagctcctcatggagcggctacaccttgaaatcaaccgccgaggccgccgggctccaccgatacttgccaatggtccagttccctctccaccccgccgaggaggggctctgctagaagaacctgagttaagtgatgatgaccgagccaacctaatgtggaaacgttacctggagcgagaggacagcaagattgtggacctgtttgtgggccagttgaaaagttgtctcaagtgccaggcctgtgggtatcgctccacgaccttcgaggttttttgtgacctgtccctgcccatccccaagaaaggatttgctgggggcaaggtgtctctgcgggattgtttcaaccttttcactaaggaagaagagctagagtcggagaatgccccagtgtgtgaccgatgtcggcagaaaactcgaagtaccaaaaagttgacagtacaaagattccctcgaatcctcgtgctccatctgaatcgattttctgcctcccgaggctccatcaaaaaaagttcagtaggtgtagactttccactgcagcgactgagcctaggggactttgccagtgacaaagccggaagtcctgtataccagctgtatgccctttgcaaccactcaggcagcgtccactatggccactacacagccctgtgccggtgccagactggttggcatgtctacaatgactctcgtgtctcccctgtcagtgaaaaccaggtggcatccagcgagggctacgtgctgttctaccaactgatgcaggagccaccccggtgcctgtgacacctctaagctctggcacctgtgaagccctttaaacacccttaagccccaggctccccgtttacctcagagacgtctatttttgtgtctttttaatcggggaggggggagggggtggttgtagctccattattttttttattaaaaaatacccttccacctggaggctcccttgtctcccagccccatgtacaaagctcaccaagcccctgcccatgtacagcccccagaccctctgcaatatcactttttgtgaataaatttattaagaaaaaaaaaaaaaaaaaaa

SEQ ID NO: 2 - DNA, 947 bp, Homo sapiens
ggctcggggccggggccagcacccacactgggtctccacagcggcatggaggcctgcgtgtcttcactgctggtgctggccctgggggccctgtcagtaggcagctcctttgggacccagatcatcgggggccgggaggtgatcccccactcgcgcccgtacatggcctcactgcagagaaatggctcccacctgtgcgggggtgtcctggtgcacccaaagtgggtgctgacggctgcccactgcctggcccagcggatggcccagctgaggctggtgctggggctccacaccctggacagccccggtctcaccttccacatcaaggcagccatccagcaccctcgctacaagcccgtccctgccctggagaacgacctcgcgctgcttcagctggacgggaaagtgaagcccagccggaccatccggccgttggccctgcccagtaagcgccaggtggtggcagcagggactcggtgcagcatggccggctgggggctgacccaccagggcgggcgcctgtcccgggtgctgcgggagctggacctccaagtgctggacacccgcatgtgtaacaacagccgcttctggaacggcagcctctcccccagcatggtctgcctggcggccgactccaaggaccaggctccctgcaagggtgactcgggcgggcccctggtgtgtggcaaaggccgggtgttggccggagtcctgtccttcagctccagggtctgcactgacatcttcaagcctcccgtggccaccgctgtggcgccttacgtgtcctggatcaggaaggtcaccggccgatcggcctgatgccctggggtgatggggaccccctcgctgtctccacaggacccttcccctccaggggtgcagtggggtgggtgaggacgggtgggagggacagggagggaccaataaatcataatgaagaaacgctc

SEQ ID NO: 3 - DNA, 2633 bp, Homo sapiens
gatttcccggggaggtcccttctgggcccccggcggaggtgggagagagtcaggcaggagccgaggccggggagccctcttcgtcagctggtgctcactgcgccgcgccagcgccagccgggactcacccgcagctccatgcttgtgcccggttcgactcgtccatcctccaagaagaggcagcccatgaggctcccagtccccactgagtgccaccctgaaggatgtcccagctctcctccaccctgaagcgctacacagaatcggcccgctacacagatgcccactatgccaagtcgggctatggtgcctacaccccgtcctcctatggggccaatctggctgcctccttactggagaaggagaaacttggtttcaagccggtccccaccagcagcttcctcacccgtccccgtacctatggcccctcctccctcctggactatgaccggggccgccccctgctgagacccgacatcactgggggtggtaagcgggcagagagccagacccggggtactgagcggcctttaggcagtggcctcagcgggggcagcggattcccttatggagtgaccaacaactgcctcagctacctgcccatcaatgcctatgaccagggggtgaccctaacccagaagctggacagccaatcagacctggcccgggatttctccagcctccggacctcagatagctaccggatagaccccaggaacctgggccgcagccccatgctggcccggacgcgcaaggagctctgcaccctgcaggggctctaccagacagccagctgccctgaatacctggtcgactacctggagaactatggtcgcaagggcagtgcatctcaggtgccctcccaggcccctccctcacgagtccctgaaatcatcagcccaacctaccgacccattggccgctacacgctgtgggagacgggaaagggtcaggcccctgggcccagccgctccagctccccgggaagagacggcatgaattctaagagtgcccagggtctggctggtcttcgaaaccttgggaacacgtgcttcatgaactcaattctgcagtgcctgagcaacactcgggagttgagagattactgcctccagaggctctacatgcgggacctgcaccacggcagcaatgcacacacagccctcgtggaagagtttgcaaaactaattcagaccatatggacttcatcccccaatgatgtggtgagcccatctgagttcaagacccagatccagagatacgcaccgcgctttgttggctataatcagcaggatgctcaggagttccttcgctttcttctggatgggctccataacgaggtgaaccgagtgacactgagacctaagtccaaccctgagaacctcgatcatcttcctgatgacgagaaaggccgacagatgtggagaaaatatctagaacgggaagacagtaggatcggggatctctttgttgggcagctaaagggctcgctgacgtgtacagattgtggttactgttctacggtcttcgaccccttctgggacctctcactgcccattgctaagcgaggttatcctgaggtgacattaatggactgcatgaggctcttcaccaaagaggatgtgcttgatggagatgaaaagccaacatgctgtcgctgccgaggcagaaaacggtgtataaagaagttctccatccagaggttcccaaagatcttggtgctccgtctgaagcggttctcagaatccaggatccgaaccagcaagctcacaacatttgtgaacttccccctaagagacctggacttaagagaatttgcctcagaaaacaccaaccatgctgtttacaacctgtacgctgtgtccaatcactccggaaccaccatgggtggccactatacagcctactgtcgcagtccagggacaggagaatggcacactttcaacgactccagcgtcactcccatgtcctccagccaagtgcgcaccagcgacgcctacctgctcttctacgaactggccagcccgccctcccgaatgtagcgccaggagccacgtcccttctcccttccccgtggtggccccgctccctaaattttttaaaaagacaaaaacaaaacaacaacaacaacacacaaacctgacaagagaaaaacaaacctgaagctgccgagcaggagtggatgcagcctgatcagggtctggagcaaggagccgggctttcctgagctgtggcccggcagggaagatcgcctggacgtggagccagcatcgccccgtgccctcggcgtttgcatttgtaaacttgtggtcttcctatgtgtcagaaacaactgtgtcttgggggggaagaccctcgctgcgccgcttcccgccgcagcgcccgcgcctccgaggggacagcgccctctggagctcgctgggagcatcaccgcctggacgcccgcgccgcggaggagccggcgcccatctccacccgcacggctcgccggtccagagccatgagccaagagccctcttcacgctgctaactccaggggacagacgaagggacatctttggaaaacgctggttttggtttttaaaaagcccaacttttttttttaatttccataactaaagtgttcagactgg

SEQ ID NO: 4 - DNA, 4307 bp, Homo sapiens
cacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatgtcccagacaggctcgcatcccgggaggggcttggcagggcgctggctgtggggagcccaaccctgcctcctgctccccattgtgccgctctcctggctggtgtggctgcttctgctactgctggcctctctcctgccctcagcccggctggccagccccctcccccgggaggaggagatcgtgtttccagagaagctcaacggcagcgtcctgcctggctcgggcacccctgccaggctgttgtgccgcttgcaggcctttggggagacgctgctactagagctggagcaggactccggtgtgcaggtcgaggggctgacagtgcagtacctgggccaggcgcctgagctgctgggtggagcagagcctggcacctacctgactggcaccatcaatggagatccggagtcggtggcatctctgcactgggatgggggagccctgttaggcgtgttacaatatcggggggctgaactccacctccagcccctggagggaggcacccctaactctgctgggggacctggggctcacatcctacgccggaagagtcctgccagcggtcaaggtcccatgtgcaacgtcaaggctcctcttggaagccccagccccagaccccgaagagccaagcgctttgcttcactgagtagatttgtggagacactggtggtggcagatgacaagatggccgcattccacggtgcggggctaaagcgctacctgctaacagtgatggcagcagcagccaaggccttcaagcacccaagcatccgcaatcctgtcagcttggtggtgactcggctagtgatcctggggtcaggcgaggaggggccccaagtggggcccagtgctgcccagaccctgcgcagcttctgtgcctggcagcggggcctcaacacccctgaggactcggaccctgaccactttgacacagccattctgtttacccgtcaggacctgtgtggagtctccacttgcgacacgctgggtatggctgatgtgggcaccgtctgtgacccggctcggagctgtgccattgtggaggatgatgggctccagtcagccttcactgctgctcatgaactgggtcatgtcttcaacatgctccatgacaactccaagccatgcatcagtttgaatgggcctttgagcacctctcgccatgtcatggcccctgtgatggctcatgtggatcctgaggagccctggtccccctgcagtgcccgcttcatcactgacttcctggacaatggctatgggcactgtctcttagacaaaccagaggctccattgcatctgcctgtgactttccctggcaaggactatgatgctgaccgccagtgccagctgaccttcgggcccgactcacgccattgtccacagctgccgccgccctgtgctgccctctggtgctctggccacctcaatggccatgccatgtgccagaccaaacactcgccctgggccgatggcacaccctgcgggcccgcacaggcctgcatgggtggtcgctgcctccacatggaccagctccaggacttcaatattccacaggctggtggctggggtccttggggaccatggggtgactgctctcggacctgtgggggtggtgtccagttctcctcccgagactgcacgaggcctgtcccccggaatggtggcaagtactgtgagggccgccgtacccgcttccgctcctgcaacactgaggactgcccaactggctcagccctgaccttccgcgaggagcagtgtgctgcctacaaccaccgcaccgacctcttcaagagcttcccagggcccatggactgggttcctcgctacacaggcgtggccccccaggaccagtgcaaactcacctgccaggcccgggcactgggctactactatgtgctggagccacgggtggtagatgggaccccctgttccccggacagctcctcggtctgtgtccagggccgatgcatccatgctggctgtgatcgcatcattggctccaagaagaagtttgacaagtgcatggtgtgcggaggggacggttctggttgcagcaagcagtcaggctccttcaggaaattcaggtacggatacaacaatgtggtcactatccccgcgggggccacccacattcttgtccggcagcagggaaaccctggccaccggagcatctacttggccctgaagctgccagatggctcctatgccctcaatggtgaatacacgctgatgccctcccccacagatgtggtactgcctggggcagtcagcttgcgctacagcggggccactgcagcctcagagacactgtcaggccatgggccactggcccagcctttgacactgcaagtcctagtggctggcaacccccaggacacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgccctttctgggcaccggggcctcggacttagctgggagaaagagagagcttctgttgctgcctcatgctaagactcagtggggaggggctgtgggcgtgagacctgcccctcctctctgccctaatgcgcaggctggccctgccctggtttcctgccctgggaggcagtgatgggttagtggatggaaggggctgacagacagccctccatctaaactgccccctctgccctgcgggtcacaggagggagggggaaggcagggagggcctgggccccagttgtatttatttagtatttattcacttttatttagcaccagggaaggggacaaggactagggtcctggggaacctgacccctgacccctcatagccctcaccctggggctaggaaatccagggtggtggtgataggtataagtggtgtgtgtatgcgtgtgtgtgtgtgtgtgaaaatgtgtgtgtgcttatgtatgaggtacaacctgttctgctttcctcttcctgaattttattttttgggaaaagaaaagtcaagggtagggtgggccttcagggagtgagggattatctttttttttttttctttctttctttcttttttttttttgagacagaatctcgctctgtcgcccaggctggagtgcaatggcacaatctcggctcactgcatcctccgcctcccgggttcaagtgattctcatgcctcagcctcctgagtagctgggattacaggctcctgccaccacgcccagctaatttttgttttgttttgtttggagacagagtctcgctattgtcaccagggctggaatgatttcagctcactgcaaccttcgccacctgggttccagcaattctcctgcctcagcctcccgagtagctgagattataggcacctaccaccacgcccggctaatttttgtatttttagtagagacggggtttcaccatgttggccaggctggtctcgaactcctgaccttaggtgatccactcgccttcatctcccaaagtgctgggattacaggcgtgagccaccgtgcctggccacgcccaactaatttttgtatttttagtagagacagggtttcaccatgttggccaggctgctcttgaactcctgacctcaggtaatcgacctgcctcggcctcccaaagtgctgggattacaggtgtgagccaccacgcccggtacatattttttaaattgaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataac

SEQ ID NO: 5 - DNA, 1540 bp, Homo sapiens
cggccgcgagtcctgcgtgatcgcgagcatgtgtgcgtgcgcgtgtttatctgaggcgcgtgcggcggccaccccagcctagtcctcttcttggtgccactggctaactaggttgagaaaccggcgccacaggcggcacacctggcccggagctggcccgctcctccccgccgagccggcgccccaacaacgcgccctctcccagtcctcacaaaggggcctagtccggcccccggctctggccgtgagggagcgctgtgggggcgcgctgccttctgcctggaagtgttgggcaggtggtgggagagcgtcaggcttgaacaacatgattttaaagcacgtgtctgtctgtcgttttttacttttagggttttggccaaattgggcgagggcacaaaataaccacttaccccttctcaccgaggaagagcgggagaaagggtatggcacagtcacaagggtgggtgaaaagatacatcaaggccttttgtaaaggcttctttgtggcggtgcctgtggcagtgactttcttggatcgggtcgcctgtgtggcaagagtagaaggagcatcgatgcagccttctttgaatcctggggggagccagtcatctgatgtggtgcttttgaaccactggaaagtgaggaattttgaagtacaccgtggtgacattgtatcattggtgtctcctaaaaacccagaacagaagatcattaagagagtgattgctcttgaaggagatattgtcagaaccataggacacaaaaaccggtatgtcaaagtcccccgtggtcacatctgggttgaaggtgatcatcatggacacagttttgacagtaattcttttgggccggtttccctaggacttctgcatgcccatgccacacatatcctgtggcccccagagcgctggcagaaattggaatctgttcttcctccagagcgcttaccagtacagagagaagaggaatgactgcatgaatctacctgagttgctggcattgggaggccagttactggaaaggaatggaaaaaagaagcctccaaaagggaaaaacttctgacaatatgatgctgtgcgagaaatatttacagcacattaaaacgatctgtattattaaataaataattttcaaatgttaaacagtattaaatggcacctgattttgtgttaaattttagttccctgttgtttaatgcccccaaaatatgcagacctttgggaatataaaaatattgcacccacatgtcttaatggggctgaatttcagattatttgttacatatacttattatattgattgttgggttttgattttggtgcttgctgctgaaataaattgaaaattaatattcaataaaaatgatgtatttgtcacttgctgttaaggtagaacaagaaggaacacatattatacacataatgatgggaattacattgtgatttgtactatctacaaaggagtaactgccagttaggaagaacagtaaacagtgatgttaaaataaaacaatgatttgtaaaaaaaaaaaaaaaaaaaa

SEQ ID NO: 6 - DNA, 4673 bp, Homo sapiens
agccagaaggatggggtggctcccactcctgctgcttctgactcaatgcttaggggtccctggtgagtgcccccaaccttgatccccatctgccttcaggaggggcttggccccattctcctattctgggatgagaaaaaagtcagggagccagaggctcagtgggcatggggcagtgaccttggcctcttgagcacagctgggaagccctaggaacacatagacatggcccacttaggcctctattagcacgtctgctctagcactgaagcagtgttaggaccacacagatgcacgcacacagcaggcagtgacccctcctgagcctgatctacccctctaacctagcgtatgcctttgtgcaggtgagagcccagctttggagtctgaatgcctagccagggcccctggctgggtaatgtgatggctctgagccttagcattctcatttgagagatgagatggggcaagctccatcacccactgctctcacagagcgtgtgtgttagatctgagcccggtgcctgggccactacacagaggcaccggtgataactaccaagtctgggcctgcttcccaggggaaattttttgacaagtatctgtgcaggggggctagactggcccttgaaagtgcatacagggtccatcccagaagccttgtagctttgatcccctgaatgaacaaactgtggacatgccaatacacattactgacatgtatgcccacctgacctgcacccactctgcagggcagcgctcgccattgaatgacttctaggtgctccggggcacagagctacagcgccgctacaagcggtggtgcccgggccttggcaggaggatgtggcagatgctgaagagtgtgctggtcgctgtgggcccttaatggactgccggtgagtggccactgggcatagataagactgggggcaggggagcctgggccgtggcgttaccttgtgccttcttctctccagggcgttccactacaatgtgagcagccatggttgccaactgctgccatggactcaacactcaccccacacgaggctgcggcattctgggcgctgtgacctcttccaggagaaaggcgagtgggggtggagaggggcagggtgggagacaggggacctcagcccaagttgatcttctgtctcttgctcccagactacatacggacctgcatcatgaacaatggggttgggtaccggggcaccatggccacgaccgtgggtggcctgtcctgccaggcttggagccacaagttcccgaacgatcaccagtgggacaaacaccttccctccgtcccggcctgggaccttcccccagcacacactatagtgatgctctgggccctcaggtacatgcccacgctccggaatggcctggaagagaacttctgccgtaaccctgatggcgaccccggaggtccttggtgccacacaacagaccctgccgtgcgcttccagagctgcggcatcaaatcctgccgggtgggtaagcggcgccgggtcaagctgggagagtggagggacaagcccacgcccatccacgaacccactggctctttgtctccagccgcgtgtgtctggtgcaatggcgaggaataccgcggcgcggtagaccgcaccgagtcagggcgcgagtgccagcgctgggatcttcagcacccgcaccagcaccccttcgagccgggcaagtacgcgtaggcggtatcggcgccctgggggccgggctagggaaggtccaggactccaggggcagggctccgtgtagggcaactgggcggggccagataagccagagtcccagggtcttcttcacgccccattaccgcccccaggttcctcgaccaaggtctggacgacaactattgccggaatcctgacggctccgagcggccatggtgctacactacggatccgcagatcgagcgagaattctgtgacctcccccgctgcggtaggcggcggggaccaggcctgggagggtacctgggaaccttggggaggggcgtggcttggccggggaggtcagaggggctgggcgtgacctgagagcatatcccgtggagtaccgtacacctgggaaaggcgggtttggtcccagccccagagggatctcagctgtcgctcggggccggacctatctcggtccatctaagggtccgaggcacagccccgccaagaggccacaagtgtcagctgcttccgcgggaagggtgagggctaccggggcacagccaataccaccaccgcgggcgtaccttgccagcgttgggacgcgcaaatcccgcatcagcaccgatttacgccagaaaaatacgcgtgcaagtgaggtgggggggcgggcgttgggacgtgctgctgcgggtgagacgggaggagggtagtcacgggcttagggctggaggctggcgggctagggctgagtgcagcgcctgcttagagaccttcgggagaacttctgccggaaccccgacggctcagaggcgccctggtgcttcacactgcggcccggcatgcgcgtgggcttttgctaccagatccggcgttgtacagacgacgtgcggccccagggtgaggcccaagcttgggggctacagagccggggctggaagcctggaaccggagggccggggcggggtctcggcctgatggctgcccgcaccggccgcagactgctaccacggcgcgggggagcagtaccgcggcacggtcagcaagacccgcaagggtgtccagtgccagcgcgggtccgctgagacgccgcacaagccgcagtgaatccctggtgctcccggccccgccagggccctaaccctggggcggcatgctttgatgtctgggaccagagcctggaaatggttgagactaccctgccacgacttcgctcccgctcccgcctcggttcacgtttacctccgaaccgcatgcacaactggaggagaacttctgccagacccagatggggatagccatgggccctggtgctacacgatggacccaaggaccccattcgactactgtgccctgcgacgctgcggtgagcactagtgacgcttgccccatgaccctgcctcagccctcaccaccaaaggctggctcccttaaccgcagtgaactttgtctttcagctgatgaccagccgccatcaatcctggacccccccaggttaggagttgggccagttatgggtcaggccctttagcccacgacatccacacagtctgggtttcatccagcccaccccatcctacagaccaggtgcagtttgagaagtgtggcaagagggtggatcggctggatcagcgttgttccaagctgcgcgtggctgggggccatccgggcaactcaccctggacagtcagcttgcggaattggtgaggcacaactgcctgtctcccacagagaggagctgaggttgtgtcctctgtggttatgccactgggggctgggaatctatccctgcccccagaggtcctagccagaagatggcaggtctagcatctgtcccaggagtctgttccctgtcctaattccccactcctctaggcagggccagcatttctgcggggggtctctagtgaaggagcagtggatactgactgcccggcagtgcttctcctcctggtgagcctcccttgtgtttggggacccagtctcatcccaccttcccctttccccaggcaagctaacaagtgagccttggggcaacggactgagagtcacaaatgacctagcagagcttctctcccagccatatgcctctcacgggctatgaggtatggttgggcaccctgttccagaacccacaacatggagagccaggcctacagcgggtcccagtagccaagatgctgtgtgggccctcaggctctcagcttgtcctgctcaagctggagaggtatgtggacaacctgggagggtgtgaggtggggctgagccttgtggcctcagaccctgagtgcccccattcttgctaaagatctgtgaccctgaaccagcgtgtggccctgatctgcctgccgcctgaatgatatgtggtgcctccagggaccaagtgtgagattgcaggccggggtgagaccaaaggtaagagcatagtgcacaggactgctggtggccaggaggcccagccctggatcttcctccaggaccgtctccttctccccattcccctcactgcaggtacgggtaatgacacagtcctaaatgtggccttgctgaatgtcatctccaaccaggagtgtaacatcaagcaccgaggacatgtgcgggagagcgagatgtgcactgagggactgttggcccctgtgggggcctgtgaggttggtggcagggccctgggccagccctggaagggtatggggggctagaaatgaactattttatcatgaagcaggctagtcatggctgtggcccagggccctcatcagttctcctacctgccagggtgactacgggggcccacttgcctgctttacccacaactgctgggtcctgaaaggaattagaatccccaaccgagtatgcgcaaggtcgcgctggccagccgtcttcacgcgtgtctctgtgtttgtggactggattcacaaggtcatgagactgggttaggcccagccttgacgccatatgctttggggaggacaaaacttgtaagtacagtca

SEQ ID NO: 7 - protein, 565 aa, Homo sapiens
MPQASEHRLGRTREPPVNIQPRVGSKLPFAPRARSKERRNPASGPNPMLRPLPPRPGLPDERLKKLELGRGRTSGPRPRGPLRADHGVPLPGSPPPTVALPLPSRTNLARSKSVSSGDLRPMGIALGGHRGTGELGAALSRLALRPEPPTLRRSTSLRRLGGFPGPPTLFSIRTEPPASHGSFHMISARSSEPFYSDDKMAHHTLLLGSGHVGLRNLGNTCFLNAVLQCLSSTRPLRDFCLRRDFRQEVPGGGRAQELTEAFADVIGALWHPDSCEAVNPTRFRAVFQKYVPSFSGYSQQDAQEFLKLLMERLHLEINRRGRRAPPILANGPVPSPPRRGGALLEEPELSDDDRANLMWKRYLEREDSKIVDLFVGQLKSCLKCQACGYRSTTFEVFCDLSLPIPKKGFAGGKVSLRDCFNLFTKEEELESENAPVCDRCRQKTRSTKKLTVQRFPRILVLHLNRFSASRGSIKKSSVGVDFPLQRLSLGDFASDKAGSPVYQLYALCNHSGSVHYGHYTALCRCQTGWHVYNDSRVSPVSENQVASSEGYVLFYQLMQEPPRCL

SEQ ID NO: 8 - protein, 257 aa, Homo sapiens
MEACVSSLLVLALGALSVGSSFGTQIIGGREVIPHSRPYMASLQRNGSHLCGGVLVHPKWVLTAAHCLAQRMAQLRLVLGLHTLDSPGLTFHIKAAIQHPRYKPVPALENDLALLQLDGKVKPSRTIRPLALPSKRQVVAAGTRCSMAGWGLTHQGGRLSRVLRELDLQVLDTRMCNNSRFWNGSLSPSMVCLAADSKDQAPCKGDSGGPLVCGKGRVLAGVLSFSSRVCTDIFKPPVATAVAPYVSWIRKVTGRSA

SEQ ID NO: 9 - protein, 605 aa, Homo sapiens
MSQLSSTLKRYTESARYTDAHYAKSGYGAYTPSSYGANLAASLLEKEKLGFKPVPTSSFLTRPRTYGPSSLLDYDRGRPLLRPDITGGGKRAESQTRGTERPLGSGLSGGSGFPYGVTNNCLSYLPINAYDQGVTLTQKLDSQSDLARDFSSLRTSDSYRIDPRNLGRSPMLARTRKELCTLQGLYQTASCPEYLVDYLENYGRKGSASQVPSQAPPSRVPEIISPTYRPIGRYTLWETGKGQAPGPSRSSSPGRDGMNSKSAQGLAGLRNLGNTCFMNSILQCLSNTRELRDYCLQRLYMRDLHHGSNAHTALVEEFAKLIQTIWTSSPNDVVSPSEFKTQIQRYAPRFVGYNQQDAQEFLRFLLDGLHNEVNRVTLRPKSNPENLDHLPDDEKGRQMWRKYLEREDSRIGDLFVGQLKGSLTCTDCGYCSTVFDPFWDLSLPIAKRGYPEVTLMDCMRLFTKEDVLDGDEKPTCCRCRGRKRCIKKFSIQRFPKILVLRLKRFSESRIRTSKLTTFVNFPLRDLDLREFASENTNHAVYNLYAVSNHSGTTMGGHYTAYCRSPGTGEWHTFNDSSVTPMSSSQVRTSDAYLLFYELASPPSRM

SEQ ID NO: 10 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGTPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQARALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNPGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 11 - protein, 175 aa, Homo sapiens
MAQSQGWVKRYIKAFCKGFFVAVPVAVTFLDRVACVARVEGASMQPSLNPGGSQSSDVVLLNHWKVRNFEVHRGDIVSLVSPKNPEQKIIKRVIALEGDIVRTIGHKNRYVKVPRGHIWVEGDHHGHSFDSNSFGPVSLGLLHAHATHILWPPERWQKLESVLPPERLPVQREEE

SEQ ID NO: 12 - protein, 648 aa, Homo sapiens
MGWLPLLLLLTQCLGVPGAPGHRATAPLQAVVPGPWQEDVADAEECAGRCGPLMDCRAFHYNVSSHGCQLLPWTQHSPHTRLRHSGRCDLFQEKGEWGYMPTLRNGLEENFCRNPDGDPGGPWCHTTDPAVRFQSCGIKSCRVAACVWCNGEEYRGAVDRTESGRECQRWDLQHPHQHPFEPGKFLDQGLDDNYCRNPDGSERPWCYTTDPQIEREFCDLPRCGSEAQPRQEATSVSCFRGKGEGYRGTANTTTAGVPCQRWDAQIPHQHRFTPEKYACKDLRENFCRNPDGSEAPWCFTLRPGMRVGFCYQIRRCTDDVRPQDCYHGAGEQYRGTVSKTRKGVQCQRGAWKWLRLPCHDFAPAPASVHVYLRTACTTGGELLPDPDGDSHGPWCYTMDPRTPFDYCALRRCDQVQFEKCGKRVDRLDQRCSKLRVAGGHPGNSPWTVSLRNWQGQHFCGGSLVKEQWILTARQCFSSCHMPLTGYEVWLGTLFQNPQHGEPGLQRVPVAKMLCGPSGSQLVLLKLERYVDNLGGWTKCEIAGRGETKGTGNDTVLNVALLNVISNQECNIKHRGHVRESEMCTEGLLAPVGACEGDYGGPLACFTHNCWVLKGIRIPNRVCARSRWPAVFTRVSVFVDWIHKVMRLG

SEQ ID NO: 13 - RNA, 7 nt, Artificial Sequence
  Linker sequence
uugcuau

SEQ ID NO: 14 - DNA, 19 bp, Artificial Sequence
  Knock-Down Sequence
cggctcaagaaactggagc

SEQ ID NO: 15 - DNA, 19 bp, Artificial Sequence
  Knock-Down Sequence
gatggctcatcacacactc

SEQ ID NO: 16 - DNA, 19 bp, Artificial Sequence
  Knock-Down Sequence
cctgggaaacacgtgcttc

SEQ ID NO: 17 - DNA, 19 bp, Artificial Sequence
  Knock-Down Sequence
acacgtgcttcctgaatgc

SEQ ID NO: 18 - DNA, 19 bp, Artificial Sequence
  Knock-Down Sequence
tcgattccgagctgtcttc

SEQ ID NO: 19 - DNA, 19 bp, Artificial Sequence
  Knock-Down Sequence
gttgtctcaagtgccaggc

SEQ ID NO: 20 - DNA, 19 bp, Artificial Sequence
  Knock-Down Sequence
agccggaagtcctgtatac

SEQ ID NO: 21 - DNA, 19 bp, Artificial Sequence
  Knock-Down Sequence
gccggaagtcctgtatacc

SEQ ID NO: 22 - DNA, 19 bp, Artificial Sequence
  Knock-Down Sequence
tggaactgggacgaggtgc

SEQ ID NO: 23 - DNA, 19 bp, Artificial Sequence
  Knock-Down Sequence
ttgatgcaggttgcaaacc

SEQ ID NO: 24 - DNA, 19 bp, Artificial Sequence
  Knock-Down Sequence
cggcctacatacccagagc

SEQ ID NO: 25 - DNA, 19 bp, Artificial Sequence
  Knock-Down Sequence
gcaagttcactacagcatc

SEQ ID NO: 26 - DNA, 19 bp, Artificial Sequence
  Knock-Down Sequence
cttccacatcaaggcagcc

SEQ ID NO: 27 - DNA, 19 bp, Homo sapiens
atcaaggcagccatccagc

SEQ ID NO: 28 - DNA, 19 bp, Artificial Sequence
  Knock-Down Sequence
ccgcatgtgtaacaacagc

SEQ ID NO: 29 - DNA, 19 bp, Artificial Sequence
  Knock-Down Sequence
tagagctggagcaggactc

SEQ ID NO: 30 - DNA, 19 bp, Artificial Sequence
  Knock-Down Sequence
tgacttcctggacaatggc

SEQ ID NO: 31 - DNA, 19 bp, Artificial Sequence
  Knock-Down Sequence
acgcctccgatacagcttc

SEQ ID NO: 32 - DNA, 19 bp, Artificial Sequence
  Knock-Down Sequence
ttctcctgcctcagcctcc

SEQ ID NO: 33 - DNA, 23 bp, Artificial Sequence
  PCR Primer
ctgcgaagctgtgaatcctactc

SEQ ID NO: 34 - DNA, 20 bp, Artificial Sequence
  PCR Primer
ggcatcctgctggctgtatc

SEQ ID NO: 35 - DNA, 18 bp, Artificial Sequence
  PCR Primer
agatacgcaccgcgcttt

SEQ ID NO: 36 - DNA, 21 bp, Artificial Sequence
  PCR Primer
tatggagcccatccagaagaa

SEQ ID NO: 37 - DNA, 24 bp, Artificial Sequence
  PCR Primer
tttgacacagccattctgtttacc

SEQ ID NO: 38 - DNA, 20 bp, Artificial Sequence
  PCR Primer
gagcccatcatcctccacaa

SEQ ID NO: 39 - DNA, 22 bp, Artificial Sequence
  PCR Primer
tacatggcctcactgcagagaa

SEQ ID NO: 40 - DNA, 19 bp, Artificial Sequence
  PCR Primer
agccgtcagcacccacttt

SEQ ID NO: 41 - DNA, 23 bp, Artificial Sequence
  PCR Primer
agcaagattgtggacctgtttgt

SEQ ID NO: 42 - DNA, 19 bp, Artificial Sequence
  PCR Primer
cgaaggtcgtggagcgata

SEQ ID NO: 43 - DNA, 23 bp, Artificial Sequence
  PCR Primer
cccactaagagacctggacttga

SEQ ID NO: 44 - DNA, 24 bp, Artificial Sequence
  PCR Primer
gattggacacagcatacaggttgt

SEQ ID NO: 45 - DNA, 18 bp, Artificial Sequence
  PCR Primer
tcccatttcccgcagaac

SEQ ID NO: 46 - DNA, 23 bp, Artificial Sequence
  PCR Primer
ttgtcatctgctaccaccagtgt

SEQ ID NO: 47 - DNA, 19 bp, Artificial Sequence
  PCR Primer
cccctgcaagggtgactct

SEQ ID NO: 48 - DNA, 23 bp, Artificial Sequence
  PCR Primer
acaggtggcttgaagatgtctgt

SEQ ID NO: 49 - RNA, 21 nt, Artificial Sequence
  siRNA Sequence
guacaaagauucccucgaauu

SEQ ID NO: 50 - RNA, 21 nt, Artificial Sequence
  siRNA Sequence
uucgagggaaucuuuguacuu

SEQ ID NO: 51 - RNA, 21 nt, Artificial Sequence
  siRNA Sequence
gaaccugaguuaagugauguu

SEQ ID NO: 52 - RNA, 21 nt, Artificial Sequence
  siRNA Sequence
caucacuuaacucagguucuu

SEQ ID NO: 53 - RNA, 21 nt, Artificial Sequence
  siRNA Sequence
gagcugucuuccagaaauauu

SEQ ID NO: 54 - RNA, 21 nt, Artificial Sequence
  siRNA Sequence
uauuucuggaagacagcucuu

SEQ ID NO: 55 - RNA, 21 nt, Artificial Sequence
  siRNA Sequence
gagcagcacucgaccucuuuu

SEQ ID NO: 56 - RNA, 21 nt, Artificial Sequence
  siRNA Sequence
aagaggucgagugcugcucuu

SEQ ID NO: 57 - RNA, 21 nt, Artificial Sequence
  siRNA Sequence
ggucugcacugacaucuucuu

SEQ ID NO: 58 - RNA, 21 nt, Artificial Sequence
  siRNA Sequence
gaagaugucagugcagaccuu

SEQ ID NO: 59 - RNA, 21 nt, Artificial Sequence
  siRNA Sequence
ggucucaccuuccacaucauu

SEQ ID NO: 60 - RNA, 21 nt, Artificial Sequence
  Knock-Down Sequence
ugauguggaaggugagaccuu

SEQ ID NO: 61 - RNA, 21 nt, Artificial Sequence
  siRNA Sequence
gcccguacauggccucacuuu

SEQ ID NO: 62 - RNA, 21 nt, Artificial Sequence
  siRNA Sequence
agugaggccauguacgggcuu

SEQ ID NO: 63 - RNA, 21 nt, Artificial Sequence
  siRNA Sequence
cgccuuacguguccuggauuu

SEQ ID NO: 64 - RNA, 21 nt, Artificial Sequence
  siRNA Sequence
auccaggacacguaaggcguu

SEQ ID NO: 65 - RNA, 22 nt, Artificial Sequence
  siRNA Sequence
cguacgcggaauacuucgauuu

SEQ ID NO: 66 - RNA, 19 nt, Artificial Sequence
  siRNA Sequence
ucgaaguauuccgcguacg

SEQ ID NO: 67 - RNA, 21 nt, Artificial Sequence
  siRNA Sequence
ggcuacguccaggagcgcacc

SEQ ID NO: 68 - RNA, 20 nt, Artificial Sequence
  siRNA Sequence
ugcgcuccuggacguagcuu

SEQ ID NO: 69 - protein, 4 aa, Artificial Sequence
  Peptide
LRGG

SEQ ID NO: 70 - protein, 4 aa, Artificial Sequence
  Peptide
  misc_feature (1)..(1): Xaa can be any naturally occurring amino acid
XPDM

SEQ ID NO: 71 - protein, 4 aa, Artificial Sequence
  Peptide
  misc_feature (1)..(1): Xaa can be any naturally occurring amino acid
XPSM

SEQ ID NO: 72 - protein, 4 aa, Artificial Sequence
  Peptide
  misc_feature (1)..(1): Xaa can be any naturally occurring amino acid
XPAM

SEQ ID NO: 73 - protein, 4 aa, Artificial Sequence
  Peptide
AAPM

SEQ ID NO: 74 - protein, 12 aa, Artificial Sequence
  Peptide
NITEGEARGSVI

SEQ ID NO: 75 - DNA, 19 bp, Artificial Sequence
  Knock-Down Sequence
tagagctggagcaggactc

SEQ ID NO: 76 - DNA, 19 bp, Artificial Sequence
  Knock-Down Sequence
ctacctgactggcaccatc

SEQ ID NO: 77 - DNA, 19 bp, Artificial Sequence
  Knock-Down Sequence
atcctacgccggaagagtc

SEQ ID NO: 78 - DNA, 19 bp, Artificial Sequence
  Knock-Down Sequence
gagccaagcgctttgcttc

SEQ ID NO: 79 - DNA, 19 bp, Artificial Sequence
  Knock-Down Sequence
tgagtagatttgtggagac

SEQ ID NO: 80 - DNA, 19 bp, Artificial Sequence
  Knock-Down Sequence
actggtggtggcagatgac

SEQ ID NO: 81 - DNA, 19 bp, Artificial Sequence
  Knock-Down Sequence
cagtgatggcagcagcagc

SEQ ID NO: 82 - DNA, 19 bp, Artificial Sequence
  Knock-Down Sequence
ggccttcaagcacccaagc

SEQ ID NO: 83 - DNA, 19 bp, Artificial Sequence
  Knock-Down Sequence
cactttgacacagccattc

SEQ ID NO: 84 - DNA, 19 bp, Artificial Sequence
  Knock-Down Sequence
acagccattctgtttaccc

SEQ ID NO: 85 - DNA, 19 bp, Artificial Sequence
  Knock-Down Sequence
catgctccatgacaactcc

SEQ ID NO: 86 - DNA, 19 bp, Artificial Sequence
  Knock-Down Sequence
tgacttcctggacaatggc

SEQ ID NO: 87 - DNA, 19 bp, Artificial Sequence
  Knock-Down Sequence
aatggctatgggcactgtc

SEQ ID NO: 88 - DNA, 19 bp, Artificial Sequence
  Knock-Down Sequence
tggctatgggcactgtctc

SEQ ID NO: 89 - DNA, 19 bp, Artificial Sequence
  Knock-Down Sequence
aaaccagaggctccattgc

SEQ ID NO: 90 - DNA, 19 bp, Artificial Sequence
  Knock-Down Sequence
ccagaggctccattgcatc

SEQ ID NO: 91 - DNA, 19 bp, Artificial Sequence
  Knock-Down Sequence
ggactatgatgctgaccgc

SEQ ID NO: 92 - DNA, 19 bp, Artificial Sequence
  Knock-Down Sequence
tcacgccattgtccacagc

SEQ ID NO: 93 - DNA, 19 bp, Artificial Sequence
  Knock-Down Sequence
tattccacaggctggtggc

SEQ ID NO: 94 - DNA, 19 bp, Artificial Sequence
  Knock-Down Sequence
cgacctcttcaagagcttc

SEQ ID NO: 95 - DNA, 19 bp, Artificial Sequence
  Knock-Down Sequence
cagtgcaaactcacctgcc

SEQ ID NO: 96 - DNA, 19 bp, Artificial Sequence
  Knock-Down Sequence
tactatgtgctggagccac

SEQ ID NO: 97 - DNA, 19 bp, Artificial Sequence
  Knock-Down Sequence
ggttctggttgcagcaagc

SEQ ID NO: 98 - DNA, 19 bp, Artificial Sequence
  Knock-Down Sequence
aacaatgtggtcactatcc

SEQ ID NO: 99 - DNA, 19 bp, Artificial Sequence
  Knock-Down Sequence
tacacgctgatgccctccc

SEQ ID NO: 100 - DNA, 19 bp, Artificial Sequence
  Knock-Down Sequence
gtcctagtggctggcaacc

SEQ ID NO: 101 - DNA, 19 bp, Artificial Sequence
  Knock-Down Sequence
acgcctccgatacagcttc

SEQ ID NO: 102 - DNA, 19 bp, Artificial Sequence
  Knock-Down Sequence
ataacctcactatcccggc

SEQ ID NO: 103 - DNA, 19 bp, Artificial Sequence
  Knock-Down Sequence
agccctccatctaaactgc

SEQ ID NO: 104 - DNA, 19 bp, Artificial Sequence
  Knock-Down Sequence
aacctgttctgctttcctc

SEQ ID NO: 105 - DNA, 19 bp, Artificial Sequence
  Knock-Down Sequence
ctgttctgctttcctcttc

SEQ ID NO: 106 - DNA, 19 bp, Artificial Sequence
  Knock-Down Sequence
agtcaagggtagggtgggc

SEQ ID NO: 107 - DNA, 19 bp, Artificial Sequence
  Knock-Down Sequence
agaatctcgctctgtcgcc

SEQ ID NO: 108 - DNA, 19 bp, Artificial Sequence
  Knock-Down Sequence
tggcacaatctcggctcac

SEQ ID NO: 109 - DNA, 19 bp, Artificial Sequence
  Knock-Down Sequence
aatctcggctcactgcatc

SEQ ID NO: 110 - DNA, 19 bp, Artificial Sequence
  Knock-Down Sequence
tcacttgaacccgggaggc

SEQ ID NO: 111 - DNA, 19 bp, Artificial Sequence
  Knock-Down Sequence
gtgattctcatgcctcagc

SEQ ID NO: 112 - DNA, 19 bp, Artificial Sequence
  Knock-Down Sequence
tcccagctactcaggaggc

SEQ ID NO: 113 - DNA, 19 bp, Artificial Sequence
  Knock-Down Sequence
tcccagctactcaggaggc

SEQ ID NO: 114 - DNA, 19 bp, Artificial Sequence
  Knock-Down Sequence
agtagctgggattacaggc

SEQ ID NO: 115 - DNA, 19 bp, Artificial Sequence
  Knock-Down Sequence
agagtctcgctattgtcac

SEQ ID NO: 116 - DNA, 19 bp, Artificial Sequence
  Knock-Down Sequence
ctgggttccagcaattctc

SEQ ID NO: 117 - DNA, 19 bp, Artificial Sequence
  Knock-Down Sequence
gcaattctcctgcctcagc

SEQ ID NO: 118 - DNA, 19 bp, Artificial Sequence
  Knock-Down Sequence
ctcctgaccttaggtgatc

SEQ ID NO: 119 - DNA, 19 bp, Artificial Sequence
  Knock-Down Sequence
tcctgaccttaggtgatcc

SEQ ID NO: 120 - DNA, 19 bp, Artificial Sequence
  Knock-Down Sequence
acgcctgtaatcccagcac

SEQ ID NO: 121 - DNA, 19 bp, Artificial Sequence
  Knock-Down Sequence
tgggattacaggcgtgagc

SEQ ID NO: 122 - DNA, 19 bp, Artificial Sequence
  Knock-Down Sequence
ggtgaaaccctgtctctac

SEQ ID NO: 123 - DNA, 19 bp, Artificial Sequence
  Knock-Down Sequence
catggtgaaaccctgtctc

SEQ ID NO: 124 - DNA, 19 bp, Artificial Sequence
  Knock-Down Sequence
catggtgaaaccctgtctc

SEQ ID NO: 125 - DNA, 19 bp, Artificial Sequence
  Knock-Down Sequence
agggtttcaccatgttggc

SEQ ID NO: 126 - DNA, 19 bp, Artificial Sequence
  Knock-Down Sequence
tggccaacatggtgaaacc

SEQ ID NO: 127 - DNA, 19 bp, Artificial Sequence
  Knock-Down Sequence
ctggccaacatggtgaaac

SEQ ID NO: 128 - DNA, 19 bp, Artificial Sequence
  Knock-Down Sequence
ctcctgacctcaggtaatc

SEQ ID NO: 129 - DNA, 19 bp, Artificial Sequence
  Knock-Down Sequence
acacctgtaatcccagcac

SEQ ID NO: 130 - DNA, 19 bp, Artificial Sequence
  Knock-Down Sequence
tcacacctgtaatcccagc

SEQ ID NO: 131 - DNA, 19 bp, Artificial Sequence
  Knock-Down Sequence
tcacacctgtaatcccagc

SEQ ID NO: 132 - DNA, 19 bp, Artificial Sequence
  Knock-Down Sequence
cactgcagagaaatggctc

SEQ ID NO: 133 - DNA, 19 bp, Artificial Sequence
  Knock-Down Sequence
cttccacatcaaggcagcc

SEQ ID NO: 134 - DNA, 19 bp, Artificial Sequence
  Knock-Down Sequence
atcaaggcagccatccagc

SEQ ID NO: 135 - DNA, 19 bp, Artificial Sequence
  Knock-Down Sequence
cacccgcatgtgtaacaac

SEQ ID NO: 136 - DNA, 19 bp, Artificial Sequence
  Knock-Down Sequence
ccgcatgtgtaacaacagc

SEQ ID NO: 137 - DNA, 19 bp, Artificial Sequence
  Knock-Down Sequence
actgacatcttcaagcctc

SEQ ID NO: 138 - DNA, 19 bp, Artificial Sequence
  Knock-Down Sequence
tgacatcttcaagcctccc

SEQ ID NO: 139 - DNA, 19 bp, Artificial Sequence
  Knock-Down Sequence
agggagggaccaataaatc

SEQ ID NO: 140 - DNA, 19 bp, Artificial Sequence
  Knock-Down Sequence
acttggtttcaagccggtc

SEQ ID NO: 141 - DNA, 19 bp, Artificial Sequence
  Knock-Down Sequence
cttggtttcaagccggtcc

SEQ ID NO: 142 - DNA, 19 bp, Artificial Sequence
  Knock-Down Sequence
ttggtttcaagccggtccc

SEQ ID NO: 143 - DNA, 19 bp, Artificial Sequence
  Knock-Down Sequence
caacaactgcctcagctac

SEQ ID NO: 144 - DNA, 19 bp, Artificial Sequence
  Knock-Down Sequence
aactgcctcagctacctgc

SEQ ID NO: 145 - DNA, 19 bp, Artificial Sequence
  Knock-Down Sequence
cctaacccagaagctggac

SEQ ID NO: 146 - DNA, 19 bp, Artificial Sequence
  Knock-Down Sequence
gctggacagccaatcagac

SEQ ID NO: 147 - DNA, 19 bp, Artificial Sequence
  Knock-Down Sequence
agccagctgccctgaatac

SEQ ID NO: 148 - DNA, 19 bp, Artificial Sequence
  Knock-Down Sequence
tacctggagaactatggtc

SEQ ID NO: 149 - DNA, 19 bp, Artificial Sequence
  Knock-Down Sequence
atcatcagcccaacctacc

SEQ ID NO: 150 - DNA, 19 bp, Artificial Sequence
  Knock-Down Sequence
ccttgggaacacgtgcttc

SEQ ID NO: 151 - DNA, 19 bp, Artificial Sequence
  Knock-Down Sequence
tcgggagttgagagattac

SEQ ID NO: 152 - DNA, 19 bp, Artificial Sequence
  Knock-Down Sequence
gacccagatccagagatac

SEQ ID NO: 153 - DNA, 19 bp, Artificial Sequence
  Knock-Down Sequence
gaggtgaaccgagtgacac

SEQ ID NO: 154 - DNA, 19 bp, Artificial Sequence
  Knock-Down Sequence
actgagacctaagtccaac

SEQ ID NO: 155 - DNA, 19 bp, Artificial Sequence
  Knock-Down Sequence
tgagacctaagtccaaccc

SEQ ID NO: 156 - DNA, 19 bp, Artificial Sequence
  Knock-Down Sequence
gtccaaccctgagaacctc

SEQ ID NO: 157 - DNA, 19 bp, Artificial Sequence
  Knock-Down Sequence
cctgagaacctcgatcatc

SEQ ID NO: 158 - DNA, 19 bp, Artificial Sequence
  Knock-Down Sequence
agggctcgctgacgtgtac

SEQ ID NO: 159 - DNA, 19 bp, Artificial Sequence
  Knock-Down Sequence
gtgtacagattgtggttac

SEQ ID NO: 160 - DNA, 19 bp, Artificial Sequence
  Knock-Down Sequence
tgttctacggtcttcgacc

SEQ ID NO: 161 - DNA, 19 bp, Artificial Sequence
  Knock-Down Sequence
gccaacatgctgtcgctgc

SEQ ID NO: 162 - DNA, 19 bp, Artificial Sequence
  Knock-Down Sequence
gttctccatccagaggttc

SEQ ID NO: 163 - DNA, 19 bp, Artificial Sequence
  Knock-Down Sequence
caccaaccatgctgtttac

SEQ ID NO: 164 - DNA, 19 bp, Artificial Sequence
  Knock-Down Sequence
caaccatgctgtttacaac

SEQ ID NO: 165 - DNA, 19 bp, Artificial Sequence
  Knock-Down Sequence
ctgtacgctgtgtccaatc

SEQ ID NO: 166 - DNA, 19 bp, Artificial Sequence
  Knock-Down Sequence
aggagaatggcacactttc

SEQ ID NO: 167 - DNA, 19 bp, Artificial Sequence
  Knock-Down Sequence
tttcaacgactccagcgtc

SEQ ID NO: 168 - DNA, 19 bp, Artificial Sequence
  Knock-Down Sequence
aacaacacacaaacctgac

SEQ ID NO: 169 - DNA, 19 bp, Artificial Sequence
  Knock-Down Sequence
aaacctgaagctgccgagc

SEQ ID NO: 170 - DNA, 19 bp, Artificial Sequence
  Knock-Down Sequence
atgttacgacctctgcctc

SEQ ID NO: 171 - DNA, 19 bp, Artificial Sequence
  Knock-Down Sequence
cggctcaagaaactggagc

SEQ ID NO: 172 - DNA, 19 bp, Artificial Sequence
  Knock-Down Sequence
aagaaactggagctgggac

SEQ ID NO: 173 - DNA, 19 bp, Artificial Sequence
  Knock-Down Sequence
aacagtggctttgcctctc

SEQ ID NO: 174 - DNA, 19 bp, Artificial Sequence
  Knock-Down Sequence
cagtggctttgcctctccc

SEQ ID NO: 175 - DNA, 19 bp, Artificial Sequence
  Knock-Down Sequence
catctcggaccaacttagc

SEQ ID NO: 176 - DNA, 19 bp, Artificial Sequence
  Knock-Down Sequence
atctcggaccaacttagcc

SEQ ID NO: 177 - DNA, 19 bp, Artificial Sequence
  Knock-Down Sequence
ggaccaacttagcccgttc

SEQ ID NO: 178 - DNA, 19 bp, Artificial Sequence
  Knock-Down Sequence
acccactttgagacgtagc

SEQ ID NO: 179 - DNA, 19 bp, Artificial Sequence
  Knock-Down Sequence
ccactttgagacgtagcac

SEQ ID NO: 180 - DNA, 19 bp, Artificial Sequence
  Knock-Down Sequence
ttcccatggctccttccac

SEQ ID NO: 181 - DNA, 19 bp, Artificial Sequence
  Knock-Down Sequence
cttccacatgatatccgcc

SEQ ID NO: 182 - DNA, 19 bp, Artificial Sequence
  Knock-Down Sequence
ttccacatgatatccgccc

SEQ ID NO: 183 - DNA, 19 bp, Artificial Sequence
  Knock-Down Sequence
tctgatgacaagatggctc

SEQ ID NO: 184 - DNA, 19 bp, Artificial Sequence
  Knock-Down Sequence
aagatggctcatcacacac

SEQ ID NO: 185 - DNA, 19 bp, Artificial Sequence
  Knock-Down Sequence
gatggctcatcacacactc

SEQ ID NO: 186 - DNA, 19 bp, Artificial Sequence
  Knock-Down Sequence
acacactccttctgggctc

SEQ ID NO: 187 - DNA, 19 bp, Artificial Sequence
  Knock-Down Sequence
tctggtcatgttggccttc

SEQ ID NO: 188 - DNA, 19 bp, Artificial Sequence
  Knock-Down Sequence
ttcgaaacctgggaaacac

SEQ ID NO: 189 - DNA, 19 bp, Artificial Sequence
  Knock-Down Sequence
cctgggaaacacgtgcttc

SEQ ID NO: 190 - DNA, 19 bp, Artificial Sequence
  Knock-Down Sequence
ctgggaaacacgtgcttcc

SEQ ID NO: 191 - DNA, 19 bp, Artificial Sequence
  Knock-Down Sequence
acacgtgcttcctgaatgc

SEQ ID NO: 192 - DNA, 19 bp, Artificial Sequence
  Knock-Down Sequence
ttcctgaatgctgtgctgc

SEQ ID NO: 193 - DNA, 19 bp, Artificial Sequence
  Knock-Down Sequence
tcgacctcttcgggacttc

SEQ ID NO: 194 - DNA, 19 bp, Artificial Sequence
  Knock-Down Sequence
tgtctgagaagggacttcc

SEQ ID NO: 195 - DNA, 19 bp, Artificial Sequence
  Knock-Down Sequence
agatgtgattggtgccctc

SEQ ID NO: 196 - DNA, 19 bp, Artificial Sequence
  Knock-Down Sequence
tcctgcgaagctgtgaatc

SEQ ID NO: 197 - DNA, 19 bp, Artificial Sequence
  Knock-Down Sequence
gaagctgtgaatcctactc

SEQ ID NO: 198 - DNA, 19 bp, Artificial Sequence
  Knock-Down Sequence
tgtgaatcctactcgattc

SEQ ID NO: 199 - DNA, 19 bp, Artificial Sequence
  Knock-Down Sequence
tactcgattccgagctgtc

SEQ ID NO: 200 - DNA, 19 bp, Artificial Sequence
  Knock-Down Sequence
tcgattccgagctgtcttc

SEQ ID NO: 201 - DNA, 19 bp, Artificial Sequence
  Knock-Down Sequence
cgatacttgccaatggtcc

SEQ ID NO: 202 - DNA, 19 bp, Artificial Sequence
  Knock-Down Sequence
ttgccaatggtccagttcc

SEQ ID NO: 203 - DNA, 19 bp, Artificial Sequence
  Knock-Down Sequence
ctaatgtggaaacgttacc

SEQ ID NO: 204 - DNA, 19 bp, Artificial Sequence
  Knock-Down Sequence
gacagcaagattgtggacc

SEQ ID NO: 205 - DNA, 19 bp, Artificial Sequence
  Knock-Down Sequence
gttgtctcaagtgccaggc

SEQ ID NO: 206 - DNA, 19 bp, Artificial Sequence
  Knock-Down Sequence
agccggaagtcctgtatac

SEQ ID NO: 207 - DNA, 19 bp, Artificial Sequence
  Knock-Down Sequence
gccggaagtcctgtatacc

SEQ ID NO: 208 - DNA, 19 bp, Artificial Sequence
  Knock-Down Sequence
tatggccactacacagccc

SEQ ID NO: 209 - DNA, 19 bp, Artificial Sequence
  Knock-Down Sequence
aatgactctcgtgtctccc

SEQ ID NO: 210 - DNA, 19 bp, Artificial Sequence
  Knock-Down Sequence
caactgatgcaggagccac

SEQ ID NO: 211 - DNA, 19 bp, Artificial Sequence
  Knock-Down Sequence
acctctaagctctggcacc

SEQ ID NO: 212 - DNA, 19 bp, Artificial Sequence
  Knock-Down Sequence
gctctggcacctgtgaagc

SEQ ID NO: 213 - DNA, 19 bp, Artificial Sequence
  Knock-Down Sequence
tacccttccacctggaggc

SEQ ID NO: 214 - DNA, 19 bp, Artificial Sequence
  Knock-Down Sequence
aagttcccgaacgatcacc

SEQ ID NO: 215 - DNA, 19 bp, Artificial Sequence
  Knock-Down Sequence
aagtgtcagctgcttccgc

SEQ ID NO: 216 - DNA, 19 bp, Artificial Sequence
  Knock-Down Sequence
ctagcagagcttctctccc

SEQ ID NO: 217 - DNA, 19 bp, Artificial Sequence
  Knock-Down Sequence
agagcgtgtgtgttagatc

SEQ ID NO: 218 - DNA, 19 bp, Artificial Sequence
  Knock-Down Sequence
cggtgataactaccaagtc

SEQ ID NO: 219 - DNA, 19 bp, Artificial Sequence
  Knock-Down Sequence
tttcttggatcgggtcgcc

SEQ ID NO: 220 - DNA, 19 bp, Artificial Sequence
  Knock-Down Sequence
accgtggtgacattgtatc

SEQ ID NO: 221 - DNA, 19 bp, Artificial Sequence
  Knock-Down Sequence
atctgggttgaaggtgatc

SEQ ID NO: 222 - DNA, 19 bp, Artificial Sequence
  Knock-Down Sequence
ttctgcatgcccatgccac

SEQ ID NO: 223 - DNA, 19 bp, Artificial Sequence
  Knock-Down Sequence
tctgttcttcctccagagc

SEQ ID NO: 224 - DNA, 21 bp, Artificial Sequence
  Knock-Down Sequence
ccatgttacgacctctgcctc

SEQ ID NO: 225 - DNA, 21 bp, Artificial Sequence
  Knock-Down Sequence
aacggctcaagaaactggagc

SEQ ID NO: 226 - DNA, 21 bp, Artificial Sequence
  Knock-Down Sequence
tcaagaaactggagctgggac

SEQ ID NO: 227 - DNA, 21 bp, Artificial Sequence
  Knock-Down Sequence
ccaacagtggctttgcctctc

SEQ ID NO: 228 - DNA, 21 bp, Artificial Sequence
  Knock-Down Sequence
aacagtggctttgcctctccc

SEQ ID NO: 229 - DNA, 21 bp, Artificial Sequence
  Knock-Down Sequence
cccatctcggaccaacttagc

SEQ ID NO: 230 - DNA, 21 bp, Artificial Sequence
  Knock-Down Sequence
ccatctcggaccaacttagcc

SEQ ID NO: 231 - DNA, 21 bp, Artificial Sequence
  Knock-Down Sequence
tcggaccaacttagcccgttc

SEQ ID NO: 232 - DNA, 21 bp, Artificial Sequence
  Knock-Down Sequence
ccacccactttgagacgtagc

SEQ ID NO: 233 - DNA, 21 bp, Artificial Sequence
  Knock-Down Sequence
acccactttgagacgtagcac

SEQ ID NO: 234 - DNA, 21 bp, Artificial Sequence
  Knock-Down Sequence
acttcccatggctccttccac

SEQ ID NO: 235 - DNA, 21 bp, Artificial Sequence
  Knock-Down Sequence
tccttccacatgatatccgcc

SEQ ID NO: 236 - DNA, 21 bp, Artificial Sequence
  Knock-Down Sequence
ccttccacatgatatccgccc

SEQ ID NO: 237 - DNA, 21 bp, Artificial Sequence
  Knock-Down Sequence
actctgatgacaagatggctc

SEQ ID NO: 238 - DNA, 21 bp, Artificial Sequence
  Knock-Down Sequence
acaagatggctcatcacacac

SEQ ID NO: 239 - DNA, 21 bp, Artificial Sequence
  Knock-Down Sequence
aagatggctcatcacacactc

SEQ ID NO: 240 - DNA, 21 bp, Artificial Sequence
  Knock-Down Sequence
tcacacactccttctgggctc

SEQ ID NO: 241 - DNA, 21 bp, Artificial Sequence
  Knock-Down Sequence
gctctggtcatgttggccttc

SEQ ID NO: 242 - DNA, 21 bp, Artificial Sequence
  Knock-Down Sequence
ccttcgaaacctgggaaacac

SEQ ID NO: 243 - DNA, 21 bp, Artificial Sequence
  Knock-Down Sequence
aacctgggaaacacgtgcttc

SEQ ID NO: 244 - DNA, 21 bp, Artificial Sequence
  Knock-Down Sequence
acctgggaaacacgtgcttcc

SEQ ID NO: 245 - DNA, 21 bp, Artificial Sequence
  Knock-Down Sequence
aaacacgtgcttcctgaatgc

SEQ ID NO: 246 - DNA, 21 bp, Artificial Sequence
  Knock-Down Sequence
gcttcctgaatgctgtgctgc

SEQ ID NO: 247 - DNA, 21 bp, Artificial Sequence
  Knock-Down Sequence
actcgacctcttcgggacttc

SEQ ID NO: 248 - DNA, 21 bp, Artificial Sequence
  Knock-Down Sequence
tctgtctgagaagggacttcc

SEQ ID NO: 249 - DNA, 21 bp, Artificial Sequence
  Knock-Down Sequence
gcagatgtgattggtgccctc

SEQ ID NO: 250 - DNA, 21 bp, Artificial Sequence
  Knock-Down Sequence
actcctgcgaagctgtgaatc

SEQ ID NO: 251 - DNA, 21 bp, Artificial Sequence
  Knock-Down Sequence
gcgaagctgtgaatcctactc

SEQ ID NO: 252 - DNA, 21 bp, Artificial Sequence
  Knock-Down Sequence
gctgtgaatcctactcgattc

SEQ ID NO: 253 - DNA, 21 bp, Artificial Sequence
  Knock-Down Sequence
cctactcgattccgagctgtc

SEQ ID NO: 254 - DNA, 21 bp, Artificial Sequence
  Knock-Down Sequence
actcgattccgagctgtcttc

SEQ ID NO: 255 - DNA, 21 bp, Artificial Sequence
  Knock-Down Sequence
accgatacttgccaatggtcc

SEQ ID NO: 256 - DNA, 21 bp, Artificial Sequence
  Knock-Down Sequence
acttgccaatggtccagttcc

SEQ ID NO: 257 - DNA, 21 bp, Artificial Sequence
  Knock-Down Sequence
acctaatgtggaaacgttacc

SEQ ID NO: 258 - DNA, 21 bp, Artificial Sequence
  Knock-Down Sequence
aagacagcaagattgtggacc

SEQ ID NO: 259 - DNA, 21 bp, Artificial Sequence
  Knock-Down Sequence
aagttgtctcaagtgccaggc

SEQ ID NO: 260 - DNA, 21 bp, Artificial Sequence
  Knock-Down Sequence
aaagccggaagtcctgtatac

SEQ ID NO: 261 - DNA, 21 bp, Artificial Sequence
  Knock-Down Sequence
aagccggaagtcctgtatacc

SEQ ID NO: 262 - DNA, 21 bp, Artificial Sequence
  Knock-Down Sequence
actatggccactacacagccc

SEQ ID NO: 263 - DNA, 21 bp, Artificial Sequence
  Knock-Down Sequence
acaatgactctcgtgtctccc

SEQ ID NO: 264 - DNA, 21 bp, Artificial Sequence
  Knock-Down Sequence
accaactgatgcaggagccac

SEQ ID NO: 265 - DNA, 21 bp, Artificial Sequence
  Knock-Down Sequence
acacctctaagctctggcacc

SEQ ID NO: 266 - DNA, 21 bp, Artificial Sequence
  Knock-Down Sequence
aagctctggcacctgtgaagc

SEQ ID NO: 267 - DNA, 21 bp, Artificial Sequence
  Knock-Down Sequence
aatacccttccacctggaggc

SEQ ID NO: 268 - DNA, 21 bp, Artificial Sequence
  Knock-Down Sequence
ccatgttacgacctctgcctc

SEQ ID NO: 269 - DNA, 21 bp, Artificial Sequence
  Knock-Down Sequence
aacggctcaagaaactggagc

SEQ ID NO: 270 - DNA, 21 bp, Artificial Sequence
  Knock-Down Sequence
tcaagaaactggagctgggac

SEQ ID NO: 271 - DNA, 21 bp, Artificial Sequence
  Knock-Down Sequence
ccaacagtggctttgcctctc

SEQ ID NO: 272 - DNA, 21 bp, Artificial Sequence
  Knock-Down Sequence
aacagtggctttgcctctccc

SEQ ID NO: 273 - DNA, 21 bp, Artificial Sequence
  Knock-Down Sequence
cccatctcggaccaacttagc

SEQ ID NO: 274 - DNA, 21 bp, Artificial Sequence
  Knock-Down Sequence
ccatctcggaccaacttagcc

SEQ ID NO: 275 - DNA, 21 bp, Artificial Sequence
  Knock-Down Sequence
tcggaccaacttagcccgttc

SEQ ID NO: 276 - DNA, 21 bp, Artificial Sequence
  Knock-Down Sequence
ccacccactttgagacgtagc

SEQ ID NO: 277 - DNA, 21 bp, Artificial Sequence
  Knock-Down Sequence
acccactttgagacgtagcac

SEQ ID NO: 278 - DNA, 21 bp, Artificial Sequence
  Knock-Down Sequence
acttcccatggctccttccac

SEQ ID NO: 279 - DNA, 21 bp, Artificial Sequence
  Knock-Down Sequence
tccttccacatgatatccgcc

SEQ ID NO: 280 - DNA, 21 bp, Artificial Sequence
  Knock-Down Sequence
ccttccacatgatatccgccc

SEQ ID NO: 281 - DNA, 21 bp, Artificial Sequence
  Knock-Down Sequence
actctgatgacaagatggctc

SEQ ID NO: 282 - DNA, 21 bp, Artificial Sequence
  Knock-Down Sequence
acaagatggctcatcacacac

SEQ ID NO: 283 - DNA, 21 bp, Artificial Sequence
  Knock-Down Sequence
aagatggctcatcacacactc

SEQ ID NO: 284 - DNA, 21 bp, Artificial Sequence
  Knock-Down Sequence
tcacacactccttctgggctc

SEQ ID NO: 285 - DNA, 21 bp, Artificial Sequence
  Knock-Down Sequence
gctctggtcatgttggccttc

SEQ ID NO: 286 - DNA, 21 bp, Artificial Sequence
  Knock-Down Sequence
ccttcgaaacctgggaaacac

SEQ ID NO: 287 - DNA, 21 bp, Artificial Sequence
  Knock-Down Sequence
aacctgggaaacacgtgcttc

SEQ ID NO: 288 - DNA, 21 bp, Artificial Sequence
  Knock-Down Sequence
acctgggaaacacgtgcttcc

SEQ ID NO: 289 - DNA, 21 bp, Artificial Sequence
  Knock-Down Sequence
aaacacgtgcttcctgaatgc

SEQ ID NO: 290 - DNA, 21 bp, Artificial Sequence
  Knock-Down Sequence
gcttcctgaatgctgtgctgc

SEQ ID NO: 291 - DNA, 21 bp, Artificial Sequence
  Knock-Down Sequence
actcgacctcttcgggacttc

SEQ ID NO: 292 - DNA, 21 bp, Artificial Sequence
  Knock-Down Sequence
tctgtctgagaagggacttcc

SEQ ID NO: 293 - DNA, 21 bp, Artificial Sequence
  Knock-Down Sequence
gcagatgtgattggtgccctc

SEQ ID NO: 294 - DNA, 21 bp, Artificial Sequence
  Knock-Down Sequence
actcctgcgaagctgtgaatc

SEQ ID NO: 295 - DNA, 21 bp, Artificial Sequence
  Knock-Down Sequence
gcgaagctgtgaatcctactc

SEQ ID NO: 296 - DNA, 21 bp, Artificial Sequence
  Knock-Down Sequence
gctgtgaatcctactcgattc

SEQ ID NO: 297 - DNA, 21 bp, Artificial Sequence
  Knock-Down Sequence
cctactcgattccgagctgtc

SEQ ID NO: 298 - DNA, 21 bp, Artificial Sequence
  Knock-Down Sequence
actcgattccgagctgtcttc

SEQ ID NO: 299 - DNA, 21 bp, Artificial Sequence
  Knock-Down Sequence
accgatacttgccaatggtcc

SEQ ID NO: 300 - DNA, 21 bp, Artificial Sequence
  Knock-Down Sequence
acttgccaatggtccagttcc

SEQ ID NO: 301 - DNA, 21 bp, Artificial Sequence
  Knock-Down Sequence
acctaatgtggaaacgttacc

SEQ ID NO: 302 - DNA, 21 bp, Artificial Sequence
  Knock-Down Sequence
aagacagcaagattgtggacc

SEQ ID NO: 303 - DNA, 21 bp, Artificial Sequence
  Knock-Down Sequence
aagttgtctcaagtgccaggc

SEQ ID NO: 304 - DNA, 21 bp, Artificial Sequence
  Knock-Down Sequence
actatggccactacacagccc

SEQ ID NO: 305 - DNA, 21 bp, Artificial Sequence
  Knock-Down Sequence
acaatgactctcgtgtctccc

SEQ ID NO: 306 - DNA, 21 bp, Artificial Sequence
  Knock-Down Sequence
accaactgatgcaggagccac

SEQ ID NO: 307 - DNA, 21 bp, Artificial Sequence
  Knock-Down Sequence
acacctctaagctctggcacc

SEQ ID NO: 308 - DNA, 21 bp, Artificial Sequence
  Knock-Down Sequence
aagctctggcacctgtgaagc

SEQ ID NO: 309 - DNA, 21 bp, Artificial Sequence
  Knock-Down Sequence
aatacccttccacctggaggc

SEQ ID NO: 310 - DNA, 19 bp, Artificial Sequence
  Knock-Down Sequence
atgttacgacctctgcctc

SEQ ID NO: 311 - DNA, 19 bp, Artificial Sequence
  Knock-Down Sequence
cggctcaagaaactggagc

SEQ ID NO: 312 - DNA, 19 bp, Artificial Sequence
  Knock-Down Sequence
aagaaactggagctgggac

SEQ ID NO: 313 - DNA, 19 bp, Artificial Sequence
  Knock-Down Sequence
aacagtggctttgcctctc

SEQ ID NO: 314 - DNA, 19 bp, Artificial Sequence
  Knock-Down Sequence
cagtggctttgcctctccc

SEQ ID NO: 315 - DNA, 19 bp, Artificial Sequence
  Knock-Down Sequence
catctcggaccaacttagc

SEQ ID NO: 316 - DNA, 19 bp, Artificial Sequence
  Knock-Down Sequence
atctcggaccaacttagcc

SEQ ID NO: 317 - DNA, 19 bp, Artificial Sequence
  Knock-Down Sequence
ggaccaacttagcccgttc

SEQ ID NO: 318 - DNA, 19 bp, Artificial Sequence
  Knock-Down Sequence
acccactttgagacgtagc

SEQ ID NO: 319 - DNA, 19 bp, Artificial Sequence
  Knock-Down Sequence
ccactttgagacgtagcac

SEQ ID NO: 320 - DNA, 19 bp, Artificial Sequence
  Knock-Down Sequence
ttcccatggctccttccac

SEQ ID NO: 321 - DNA, 19 bp, Artificial Sequence
  Knock-Down Sequence
cttccacatgatatccgcc

SEQ ID NO: 322 - DNA, 19 bp, Artificial Sequence
  Knock-Down Sequence
ttccacatgatatccgccc

SEQ ID NO: 323 - DNA, 19 bp, Artificial Sequence
  Knock-Down Sequence
tctgatgacaagatggctc

SEQ ID NO: 324 - DNA, 19 bp, Artificial Sequence
  Knock-Down Sequence
aagatggctcatcacacac

SEQ ID NO: 325 - DNA, 19 bp, Artificial Sequence
  Knock-Down Sequence
gatggctcatcacacactc

SEQ ID NO: 326 - DNA, 19 bp, Artificial Sequence
  Knock-Down Sequence
acacactccttctgggctc

SEQ ID NO: 327 - DNA, 19 bp, Artificial Sequence
  Knock-Down Sequence
tctggtcatgttggccttc

SEQ ID NO: 328 - DNA, 19 bp, Artificial Sequence
  Knock-Down Sequence
ttcgaaacctgggaaacac

SEQ ID NO: 329 - DNA, 19 bp, Artificial Sequence
  Knock-Down Sequence
cctgggaaacacgtgcttc

SEQ ID NO: 330 - DNA, 19 bp, Artificial Sequence
  Knock-Down Sequence
ctgggaaacacgtgcttcc

SEQ ID NO: 331 - DNA, 19 bp, Artificial Sequence
  Knock-Down Sequence
acacgtgcttcctgaatgc

SEQ ID NO: 332 - DNA, 19 bp, Artificial Sequence
  Knock-Down Sequence
ttcctgaatgctgtgctgc

SEQ ID NO: 333 - DNA, 19 bp, Artificial Sequence
  Knock-Down Sequence
tcgacctcttcgggacttc

SEQ ID NO: 334 - DNA, 19 bp, Artificial Sequence
  Knock-Down Sequence
tgtctgagaagggacttcc

SEQ ID NO: 335 - DNA, 19 bp, Artificial Sequence
  Knock-Down Sequence
agatgtgattggtgccctc

SEQ ID NO: 336 - DNA, 19 bp, Artificial Sequence
  Knock-Down Sequence
tcctgcgaagctgtgaatc

SEQ ID NO: 337 - DNA, 19 bp, Artificial Sequence
  Knock-Down Sequence
gaagctgtgaatcctactc

SEQ ID NO: 338 - DNA, 19 bp, Artificial Sequence
  Knock-Down Sequence
tgtgaatcctactcgattc

SEQ ID NO: 339 - DNA, 19 bp, Artificial Sequence
  Knock-Down Sequence
tactcgattccgagctgtc

SEQ ID NO: 340 - DNA, 19 bp, Artificial Sequence
  Knock-Down Sequence
tcgattccgagctgtcttc

SEQ ID NO: 341 - DNA, 19 bp, Artificial Sequence
  Knock-Down Sequence
cgatacttgccaatggtcc

SEQ ID NO: 342 - DNA, 19 bp, Artificial Sequence
  Knock-Down Sequence
ttgccaatggtccagttcc

SEQ ID NO: 343 - DNA, 19 bp, Artificial Sequence
  Knock-Down Sequence
ctaatgtggaaacgttacc

SEQ ID NO: 344 - DNA, 19 bp, Artificial Sequence
  Knock-Down Sequence
gacagcaagattgtggacc

SEQ ID NO: 345 - DNA, 19 bp, Artificial Sequence
  Knock-Down Sequence
gttgtctcaagtgccaggc

SEQ ID NO: 346 - DNA, 19 bp, Artificial Sequence
  Knock-Down Sequence
agccggaagtcctgtatac

SEQ ID NO: 347 - DNA, 19 bp, Artificial Sequence
  Knock-Down Sequence
gccggaagtcctgtatacc

SEQ ID NO: 348 - DNA, 19 bp, Artificial Sequence
  Knock-Down Sequence
tatggccactacacagccc

SEQ ID NO: 349 - DNA, 19 bp, Artificial Sequence
  Knock-Down Sequence
aatgactctcgtgtctccc

SEQ ID NO: 350 - DNA, 19 bp, Artificial Sequence
  Knock-Down Sequence
caactgatgcaggagccac

SEQ ID NO: 351 - DNA, 19 bp, Artificial Sequence
  Knock-Down Sequence
acctctaagctctggcacc

SEQ ID NO: 352 - DNA, 19 bp, Artificial Sequence
  Knock-Down Sequence
gctctggcacctgtgaagc

SEQ ID NO: 353 - DNA, 19 bp, Artificial Sequence
  Knock-Down Sequence
tacccttccacctggaggc

SEQ ID NO: 354 - DNA, 19 bp, Artificial Sequence
  Knock-Down Sequence
atgttacgacctctgcctc

SEQ ID NO: 355 - DNA, 19 bp, Artificial Sequence
  Knock-Down Sequence
cggctcaagaaactggagc

SEQ ID NO: 356 - DNA, 19 bp, Artificial Sequence
  Knock-Down Sequence
aagaaactggagctgggac

SEQ ID NO: 357 - DNA, 19 bp, Artificial Sequence
  Knock-Down Sequence
aacagtggctttgcctctc

SEQ ID NO: 358 - DNA, 19 bp, Artificial Sequence
  Knock-Down Sequence
cagtggctttgcctctccc

SEQ ID NO: 359 - DNA, 19 bp, Artificial Sequence
  Knock-Down Sequence
catctcggaccaacttagc

SEQ ID NO: 360 - DNA, 19 bp, Artificial Sequence
  Knock-Down Sequence
atctcggaccaacttagcc

SEQ ID NO: 361 - DNA, 19 bp, Artificial Sequence
  Knock-Down Sequence
ggaccaacttagcccgttc

SEQ ID NO: 362 - DNA, 19 bp, Artificial Sequence
  Knock-Down Sequence
acccactttgagacgtagc

SEQ ID NO: 363 - DNA, 19 bp, Artificial Sequence
  Knock-Down Sequence
ccactttgagacgtagcac

SEQ ID NO: 364 - DNA, 19 bp, Artificial Sequence
  Knock-Down Sequence
ttcccatggctccttccac

SEQ ID NO: 365 - DNA, 19 bp, Artificial Sequence
  Knock-Down Sequence
cttccacatgatatccgcc

SEQ ID NO: 366 - DNA, 19 bp, Artificial Sequence
  Knock-Down Sequence
ttccacatgatatccgccc

SEQ ID NO: 367 - DNA, 19 bp, Artificial Sequence
  Knock-Down Sequence
tctgatgacaagatggctc

SEQ ID NO: 368 - DNA, 19 bp, Artificial Sequence
  Knock-Down Sequence
aagatggctcatcacacac

SEQ ID NO: 369 - DNA, 19 bp, Artificial Sequence
  Knock-Down Sequence
gatggctcatcacacactc

SEQ ID NO: 370 - DNA, 19 bp, Artificial Sequence
  Knock-Down Sequence
acacactccttctgggctc

SEQ ID NO: 371 - DNA, 19 bp, Artificial Sequence
  Knock-Down Sequence
tctggtcatgttggccttc

SEQ ID NO: 372 - DNA, 19 bp, Artificial Sequence
  Knock-Down Sequence
ttcgaaacctgggaaacac

SEQ ID NO: 373 - DNA, 19 bp, Artificial Sequence
  Knock-Down Sequence
cctgggaaacacgtgcttc

SEQ ID NO: 374 - DNA, 19 bp, Artificial Sequence
  Knock-Down Sequence
ctgggaaacacgtgcttcc

SEQ ID NO: 375 - DNA, 19 bp, Artificial Sequence
  Knock-Down Sequence
acacgtgcttcctgaatgc

SEQ ID NO: 376 - DNA, 19 bp, Artificial Sequence
  Knock-Down Sequence
ttcctgaatgctgtgctgc

SEQ ID NO: 377 - DNA, 19 bp, Artificial Sequence
  Knock-Down Sequence
tcgacctcttcgggacttc

SEQ ID NO: 378 - DNA, 19 bp, Artificial Sequence
  Knock-Down Sequence
tgtctgagaagggacttcc

SEQ ID NO: 379 - DNA, 19 bp, Artificial Sequence
  Knock-Down Sequence
agatgtgattggtgccctc

SEQ ID NO: 380 - DNA, 19 bp, Artificial Sequence
  Knock-Down Sequence
tcctgcgaagctgtgaatc

SEQ ID NO: 381 - DNA, 19 bp, Artificial Sequence
  Knock-Down Sequence
gaagctgtgaatcctactc

SEQ ID NO: 382 - DNA, 19 bp, Artificial Sequence
  Knock-Down Sequence
tgtgaatcctactcgattc

SEQ ID NO: 383 - DNA, 19 bp, Artificial Sequence
  Knock-Down Sequence
tactcgattccgagctgtc

SEQ ID NO: 384 - DNA, 19 bp, Artificial Sequence
  Knock-Down Sequence
tcgattccgagctgtcttc

SEQ ID NO: 385 - DNA, 19 bp, Artificial Sequence
  Knock-Down Sequence
cgatacttgccaatggtcc

SEQ ID NO: 386 - DNA, 19 bp, Artificial Sequence
  Knock-Down Sequence
ttgccaatggtccagttcc

SEQ ID NO: 387 - DNA, 19 bp, Artificial Sequence
  Knock-Down Sequence
ctaatgtggaaacgttacc

SEQ ID NO: 388 - DNA, 19 bp, Artificial Sequence
  Knock-Down Sequence
gacagcaagattgtggacc

SEQ ID NO: 389 - DNA, 19 bp, Artificial Sequence
  Knock-Down Sequence
gttgtctcaagtgccaggc

SEQ ID NO: 390 - DNA, 19 bp, Artificial Sequence
  Knock-Down Sequence
tatggccactacacagccc

SEQ ID NO: 391 - DNA, 19 bp, Artificial Sequence
  Knock-Down Sequence
aatgactctcgtgtctccc

SEQ ID NO: 392 - DNA, 19 bp, Artificial Sequence
  Knock-Down Sequence
caactgatgcaggagccac

SEQ ID NO: 393 - DNA, 19 bp, Artificial Sequence
  Knock-Down Sequence
acctctaagctctggcacc

SEQ ID NO: 394 - DNA, 19 bp, Artificial Sequence
  Knock-Down Sequence
gctctggcacctgtgaagc

SEQ ID NO: 395 - DNA, 19 bp, Artificial Sequence
  Knock-Down Sequence
tacccttccacctggaggc

SEQ ID NO: 396 - DNA, 21 bp, Artificial Sequence
  Knock-Down Sequence
ctcactgcagagaaatggctc

SEQ ID NO: 397 - DNA, 21 bp, Artificial Sequence
  Knock-Down Sequence
accttccacatcaaggcagcc

SEQ ID NO: 398 - DNA, 21 bp, Artificial Sequence
  Knock-Down Sequence
acatcaaggcagccatccagc

SEQ ID NO: 399 - DNA, 21 bp, Artificial Sequence
  Knock-Down Sequence
gacacccgcatgtgtaacaac

SEQ ID NO: 400 - DNA, 21 bp, Artificial Sequence
  Knock-Down Sequence
acccgcatgtgtaacaacagc

SEQ ID NO: 401 - DNA, 21 bp, Artificial Sequence
  Knock-Down Sequence
gcactgacatcttcaagcctc

SEQ ID NO: 402 - DNA, 21 bp, Artificial Sequence
  Knock-Down Sequence
actgacatcttcaagcctccc

SEQ ID NO: 403 - DNA, 21 bp, Artificial Sequence
  Knock-Down Sequence
acagggagggaccaataaatc

SEQ ID NO: 404 - DNA, 19 bp, Artificial Sequence
  Knock-Down Sequence
cactgcagagaaatggctc

SEQ ID NO: 405 - DNA, 19 bp, Artificial Sequence
  Knock-Down Sequence
cttccacatcaaggcagcc

SEQ ID NO: 406 - DNA, 19 bp, Artificial Sequence
  Knock-Down Sequence
atcaaggcagccatccagc

SEQ ID NO: 407 - DNA, 19 bp, Artificial Sequence
  Knock-Down Sequence
cacccgcatgtgtaacaac

SEQ ID NO: 408 - DNA, 19 bp, Artificial Sequence
  Knock-Down Sequence
ccgcatgtgtaacaacagc

SEQ ID NO: 409 - DNA, 19 bp, Artificial Sequence
  Knock-Down Sequence
actgacatcttcaagcctc

SEQ ID NO: 410 - DNA, 19 bp, Artificial Sequence
  Knock-Down Sequence
tgacatcttcaagcctccc

SEQ ID NO: 411 - DNA, 19 bp, Artificial Sequence
  Knock-Down Sequence
agggagggaccaataaatc

SEQ ID NO: 412 - DNA, 21 bp, Artificial Sequence
  Knock-Down Sequence
aaacttggtttcaagccggtc

SEQ ID NO: 413 - DNA, 21 bp, Artificial Sequence
  Knock-Down Sequence
aacttggtttcaagccggtcc

SEQ ID NO: 414 - DNA, 21 bp, Artificial Sequence
  Knock-Down Sequence
acttggtttcaagccggtccc

SEQ ID NO: 415 - DNA, 21 bp, Artificial Sequence
  Knock-Down Sequence
accaacaactgcctcagctac

SEQ ID NO: 416 - DNA, 21 bp, Artificial Sequence
  Knock-Down Sequence
acaactgcctcagctacctgc

SEQ ID NO: 417 - DNA, 21 bp, Artificial Sequence
  Knock-Down Sequence
accctaacccagaagctggac

SEQ ID NO: 418 - DNA, 21 bp, Artificial Sequence
  Knock-Down Sequence
aagctggacagccaatcagac

SEQ ID NO: 419 - DNA, 21 bp, Artificial Sequence
  Knock-Down Sequence
acagccagctgccctgaatac

SEQ ID NO: 420 - DNA, 21 bp, Artificial Sequence
  Knock-Down Sequence
actacctggagaactatggtc

SEQ ID NO: 421 - DNA, 21 bp, Artificial Sequence
  Knock-Down Sequence
aaatcatcagcccaacctacc

SEQ ID NO: 422 - DNA, 21 bp, Artificial Sequence
  Knock-Down Sequence
aaccttgggaacacgtgcttc

SEQ ID NO: 423 - DNA, 21 bp, Artificial Sequence
  Knock-Down Sequence
actcgggagttgagagattac

SEQ ID NO: 424 - DNA, 21 bp, Artificial Sequence
  Knock-Down Sequence
aagacccagatccagagatac

SEQ ID NO: 425 - DNA, 21 bp, Artificial Sequence
  Knock-Down Sequence
acgaggtgaaccgagtgacac

SEQ ID NO: 426 - DNA, 21 bp, Artificial Sequence
  Knock-Down Sequence
acactgagacctaagtccaac

SEQ ID NO: 427 - DNA, 21 bp, Artificial Sequence
  Knock-Down Sequence
actgagacctaagtccaaccc

SEQ ID NO: 428 - DNA, 21 bp, Artificial Sequence
  Knock-Down Sequence
aagtccaaccctgagaacctc

SEQ ID NO: 429 - DNA, 21 bp, Artificial Sequence
  Knock-Down Sequence
accctgagaacctcgatcatc

SEQ ID NO: 430 - DNA, 21 bp, Artificial Sequence
  Knock-Down Sequence
aaagggctcgctgacgtgtac

SEQ ID NO: 431 - DNA, 21 bp, Artificial Sequence
  Knock-Down Sequence
acgtgtacagattgtggttac

SEQ ID NO: 432 - DNA, 21 bp, Artificial Sequence
  Knock-Down Sequence
actgttctacggtcttcgacc

SEQ ID NO: 433 - DNA, 21 bp, Artificial Sequence
  Knock-Down Sequence
aagccaacatgctgtcgctgc

SEQ ID NO: 434 - DNA, 21 bp, Artificial Sequence
  Knock-Down Sequence
aagttctccatccagaggttc

SEQ ID NO: 435 - DNA, 21 bp, Artificial Sequence
  Knock-Down Sequence
aacaccaaccatgctgtttac

SEQ ID NO: 436 - DNA, 21 bp, Artificial Sequence
  Knock-Down Sequence
accaaccatgctgtttacaac

SEQ ID NO: 437 - DNA, 21 bp, Artificial Sequence
  Knock-Down Sequence
acctgtacgctgtgtccaatc

SEQ ID NO: 438 - DNA, 21 bp, Artificial Sequence
  Knock-Down Sequence
acaggagaatggcacactttc

SEQ ID NO: 439 - DNA, 21 bp, Artificial Sequence
  Knock-Down Sequence
actttcaacgactccagcgtc

SEQ ID NO: 440 - DNA, 21 bp, Artificial Sequence
  Knock-Down Sequence
acaacaacacacaaacctgac

SEQ ID NO: 441 - DNA, 21 bp, Artificial Sequence
  Knock-Down Sequence
acaaacctgaagctgccgagc

SEQ ID NO: 442 - DNA, 21 bp, Artificial Sequence
  Knock-Down Sequence
aaccttgggaacacgtgcttc

SEQ ID NO: 443 - DNA, 21 bp, Artificial Sequence
  Knock-Down Sequence
actcgggagttgagagattac

SEQ ID NO: 444 - DNA, 21 bp, Artificial Sequence
  Knock-Down Sequence
aagacccagatccagagatac

SEQ ID NO: 445 - DNA, 21 bp, Artificial Sequence
  Knock-Down Sequence
acgaggtgaaccgagtgacac

SEQ ID NO: 446 - DNA, 21 bp, Artificial Sequence
  Knock-Down Sequence
acactgagacctaagtccaac

SEQ ID NO: 447 - DNA, 21 bp, Artificial Sequence
  Knock-Down Sequence
actgagacctaagtccaaccc

SEQ ID NO: 448 - DNA, 21 bp, Artificial Sequence
  Knock-Down Sequence
aagtccaaccctgagaacctc

SEQ ID NO: 449 - DNA, 21 bp, Artificial Sequence
  Knock-Down Sequence
accctgagaacctcgatcatc

SEQ ID NO: 450 - DNA, 21 bp, Artificial Sequence
  Knock-Down Sequence
acgtgtacagattgtggttac

SEQ ID NO: 451 - DNA, 21 bp, Artificial Sequence
  Knock-Down Sequence
actgttctacggtcttcgacc

SEQ ID NO: 452 - DNA, 21 bp, Artificial Sequence
  Knock-Down Sequence
aagccaacatgctgtcgctgc

SEQ ID NO: 453 - DNA, 21 bp, Artificial Sequence
  Knock-Down Sequence
aagttctccatccagaggttc

SEQ ID NO: 454 - DNA, 21 bp, Artificial Sequence
  Knock-Down Sequence
aacaccaaccatgctgtttac

SEQ ID NO: 455 - DNA, 21 bp, Artificial Sequence
  Knock-Down Sequence
accaaccatgctgtttacaac

SEQ ID NO: 456 - DNA, 21 bp, Artificial Sequence
  Knock-Down Sequence
acctgtacgctgtgtccaatc

SEQ ID NO: 457 - DNA, 21 bp, Artificial Sequence
  Knock-Down Sequence
acaggagaatggcacactttc

SEQ ID NO: 458 - DNA, 21 bp, Artificial Sequence
  Knock-Down Sequence
actttcaacgactccagcgtc

SEQ ID NO: 459 - DNA, 19 bp, Artificial Sequence
  Knock-Down Sequence
acttggtttcaagccggtc

SEQ ID NO: 460 - DNA, 19 bp, Artificial Sequence
  Knock-Down Sequence
cttggtttcaagccggtcc

SEQ ID NO: 461 - DNA, 19 bp, Artificial Sequence
  Knock-Down Sequence
ttggtttcaagccggtccc

SEQ ID NO: 462 - DNA, 19 bp, Artificial Sequence
  Knock-Down Sequence
caacaactgcctcagctac

SEQ ID NO: 463 - DNA, 19 bp, Artificial Sequence
  Knock-Down Sequence
aactgcctcagctacctgc

SEQ ID NO: 464 - DNA, 19 bp, Artificial Sequence
  Knock-Down Sequence
cctaacccagaagctggac

SEQ ID NO: 465 - DNA, 19 bp, Artificial Sequence
  Knock-Down Sequence
gctggacagccaatcagac

SEQ ID NO: 466 - DNA, 19 bp, Artificial Sequence
  Knock-Down Sequence
agccagctgccctgaatac

SEQ ID NO: 467 - DNA, 19 bp, Artificial Sequence
  Knock-Down Sequence
tacctggagaactatggtc

SEQ ID NO: 468 - DNA, 19 bp, Artificial Sequence
  Knock-Down Sequence
atcatcagcccaacctacc

SEQ ID NO: 469 - DNA, 19 bp, Artificial Sequence
  Knock-Down Sequence
ccttgggaacacgtgcttc

SEQ ID NO: 470 - DNA, 19 bp, Artificial Sequence
  Knock-Down Sequence
tcgggagttgagagattac

SEQ ID NO: 471 - DNA, 19 bp, Artificial Sequence
  Knock-Down Sequence
gacccagatccagagatac

SEQ ID NO: 472 - DNA, 19 bp, Artificial Sequence
  Knock-Down Sequence
gaggtgaaccgagtgacac

SEQ ID NO: 473 - DNA, 19 bp, Artificial Sequence
  Knock-Down Sequence
actgagacctaagtccaac

SEQ ID NO: 474 - DNA, 19 bp, Artificial Sequence
  Knock-Down Sequence
tgagacctaagtccaaccc

SEQ ID NO: 475 - DNA, 19 bp, Artificial Sequence
  Knock-Down Sequence
gtccaaccctgagaacctc

SEQ ID NO: 476 - DNA, 19 bp, Artificial Sequence
  Knock-Down Sequence
cctgagaacctcgatcatc

SEQ ID NO: 477 - DNA, 19 bp, Artificial Sequence
  Knock-Down Sequence
agggctcgctgacgtgtac

SEQ ID NO: 478 - DNA, 19 bp, Artificial Sequence
  Knock-Down Sequence
gtgtacagattgtggttac

SEQ ID NO: 479 - DNA, 19 bp, Artificial Sequence
  Knock-Down Sequence
tgttctacggtcttcgacc

SEQ ID NO: 480 - DNA, 19 bp, Artificial Sequence
  Knock-Down Sequence
gccaacatgctgtcgctgc

SEQ ID NO: 481 - DNA, 19 bp, Artificial Sequence
  Knock-Down Sequence
gttctccatccagaggttc

SEQ ID NO: 482 - DNA, 19 bp, Artificial Sequence
  Knock-Down Sequence
caccaaccatgctgtttac

SEQ ID NO: 483 - DNA, 19 bp, Artificial Sequence
  Knock-Down Sequence
caaccatgctgtttacaac

SEQ ID NO: 484 - DNA, 19 bp, Artificial Sequence
  Knock-Down Sequence
ctgtacgctgtgtccaatc

SEQ ID NO: 485 - DNA, 19 bp, Artificial Sequence
  Knock-Down Sequence
aggagaatggcacactttc

SEQ ID NO: 486 - DNA, 19 bp, Artificial Sequence
  Knock-Down Sequence
tttcaacgactccagcgtc

SEQ ID NO: 487 - DNA, 19 bp, Artificial Sequence
  Knock-Down Sequence
aacaacacacaaacctgac

SEQ ID NO: 488 - DNA, 19 bp, Artificial Sequence
  Knock-Down Sequence
aaacctgaagctgccgagc

SEQ ID NO: 489 - DNA, 19 bp, Artificial Sequence
  Knock-Down Sequence
ccttgggaacacgtgcttc

SEQ ID NO: 490 - DNA, 19 bp, Artificial Sequence
  Knock-Down Sequence
tcgggagttgagagattac

SEQ ID NO: 491 - DNA, 19 bp, Artificial Sequence
  Knock-Down Sequence
gacccagatccagagatac

SEQ ID NO: 492 - DNA, 19 bp, Artificial Sequence
  Knock-Down Sequence
gaggtgaaccgagtgacac

SEQ ID NO: 493 - DNA, 19 bp, Artificial Sequence
  Knock-Down Sequence
actgagacctaagtccaac

SEQ ID NO: 494 - DNA, 19 bp, Artificial Sequence
  Knock-Down Sequence
tgagacctaagtccaaccc

SEQ ID NO: 495 - DNA, 19 bp, Artificial Sequence
  Knock-Down Sequence
gtccaaccctgagaacctc

SEQ ID NO: 496 - DNA, 19 bp, Artificial Sequence
  Knock-Down Sequence
cctgagaacctcgatcatc

SEQ ID NO: 497 - DNA, 19 bp, Artificial Sequence
  Knock-Down Sequence
gtgtacagattgtggttac

SEQ ID NO: 498 - DNA, 19 bp, Artificial Sequence
  Knock-Down Sequence
tgttctacggtcttcgacc

SEQ ID NO: 499 - DNA, 19 bp, Artificial Sequence
  Knock-Down Sequence
gccaacatgctgtcgctgc

SEQ ID NO: 500 - DNA, 19 bp, Artificial Sequence
  Knock-Down Sequence
gttctccatccagaggttc

SEQ ID NO: 501 - DNA, 19 bp, Artificial Sequence
  Knock-Down Sequence
caccaaccatgctgtttac

SEQ ID NO: 502 - DNA, 19 bp, Artificial Sequence
  Knock-Down Sequence
caaccatgctgtttacaac

SEQ ID NO: 503 - DNA, 19 bp, Artificial Sequence
  Knock-Down Sequence
ctgtacgctgtgtccaatc

SEQ ID NO: 504 - DNA, 19 bp, Artificial Sequence
  Knock-Down Sequence
aggagaatggcacactttc

SEQ ID NO: 505 - DNA, 19 bp, Artificial Sequence
  Knock-Down Sequence
tttcaacgactccagcgtc

SEQ ID NO: 506 - DNA, 21 bp, Artificial Sequence
  Knock-Down Sequence
actagagctggagcaggactc

SEQ ID NO: 507 - DNA, 21 bp, Artificial Sequence
  Knock-Down Sequence
acctacctgactggcaccatc

SEQ ID NO: 508 - DNA, 21 bp, Artificial Sequence
  Knock-Down Sequence
acatcctacgccggaagagtc

SEQ ID NO: 509 - DNA, 21 bp, Artificial Sequence
  Knock-Down Sequence
aagagccaagcgctttgcttc

SEQ ID NO: 510 - DNA, 21 bp, Artificial Sequence
  Knock-Down Sequence
actgagtagatttgtggagac

SEQ ID NO: 511 - DNA, 21 bp, Artificial Sequence
  Knock-Down Sequence
acactggtggtggcagatgac

SEQ ID NO: 512 - DNA, 21 bp, Artificial Sequence
  Knock-Down Sequence
aacagtgatggcagcagcagc

SEQ ID NO: 513 - DNA, 21 bp, Artificial Sequence
  Knock-Down Sequence
aaggccttcaagcacccaagc

SEQ ID NO: 514 - DNA, 21 bp, Artificial Sequence
  Knock-Down Sequence
accactttgacacagccattc

SEQ ID NO: 515 - DNA, 21 bp, Artificial Sequence
  Knock-Down Sequence
acacagccattctgtttaccc

SEQ ID NO: 516 - DNA, 21 bp, Artificial Sequence
  Knock-Down Sequence
aacatgctccatgacaactcc

SEQ ID NO: 517 - DNA, 21 bp, Artificial Sequence
  Knock-Down Sequence
actgacttcctggacaatggc

SEQ ID NO: 518 - DNA, 21 bp, Artificial Sequence
  Knock-Down Sequence
acaatggctatgggcactgtc

SEQ ID NO: 519 - DNA, 21 bp, Artificial Sequence
  Knock-Down Sequence
aatggctatgggcactgtctc

SEQ ID NO: 520 - DNA, 21 bp, Artificial Sequence
  Knock-Down Sequence
acaaaccagaggctccattgc

SEQ ID NO: 521 - DNA, 21 bp, Artificial Sequence
  Knock-Down Sequence
aaccagaggctccattgcatc

SEQ ID NO: 522 - DNA, 21 bp, Artificial Sequence
  Knock-Down Sequence
aaggactatgatgctgaccgc

SEQ ID NO: 523 - DNA, 21 bp, Artificial Sequence
  Knock-Down Sequence
actcacgccattgtccacagc

SEQ ID NO: 524 - DNA, 21 bp, Artificial Sequence
  Knock-Down Sequence
aatattccacaggctggtggc

SEQ ID NO: 525 - DNA, 21 bp, Artificial Sequence
  Knock-Down Sequence
accgacctcttcaagagcttc

SEQ ID NO: 526 - DNA, 21 bp, Artificial Sequence
  Knock-Down Sequence
accagtgcaaactcacctgcc

SEQ ID NO: 527 - DNA, 21 bp, Artificial Sequence
  Knock-Down Sequence
actactatgtgctggagccac

SEQ ID NO: 528 - DNA, 21 bp, Artificial Sequence
  Knock-Down Sequence
acggttctggttgcagcaagc

SEQ ID NO: 529 - DNA, 21 bp, Artificial Sequence
  Knock-Down Sequence
acaacaatgtggtcactatcc

SEQ ID NO: 530 - DNA, 21 bp, Artificial Sequence
  Knock-Down Sequence
aatacacgctgatgccctccc

SEQ ID NO: 531 - DNA, 21 bp, Artificial Sequence
  Knock-Down Sequence
aagtcctagtggctggcaacc

SEQ ID NO: 532 - DNA, 21 bp, Artificial Sequence
  Knock-Down Sequence
acacgcctccgatacagcttc

SEQ ID NO: 533 - DNA, 21 bp, Artificial Sequence
  Knock-Down Sequence
aaataacctcactatcccggc

SEQ ID NO: 534 - DNA, 21 bp, Artificial Sequence
  Knock-Down Sequence
acagccctccatctaaactgc

SEQ ID NO: 535 - DNA, 21 bp, Artificial Sequence
  Knock-Down Sequence
acaacctgttctgctttcctc

SEQ ID NO: 536 - DNA, 21 bp, Artificial Sequence
  Knock-Down Sequence
acctgttctgctttcctcttc

SEQ ID NO: 537 - DNA, 21 bp, Artificial Sequence
  Knock-Down Sequence
aaagtcaagggtagggtgggc

SEQ ID NO: 538 - DNA, 21 bp, Artificial Sequence
  Knock-Down Sequence
acagaatctcgctctgtcgcc

SEQ ID NO: 539 - DNA, 21 bp, Artificial Sequence
  Knock-Down Sequence
aatggcacaatctcggctcac

SEQ ID NO: 540 - DNA, 21 bp, Artificial Sequence
  Knock-Down Sequence
acaatctcggctcactgcatc

SEQ ID NO: 541 - DNA, 21 bp, Artificial Sequence
  Knock-Down Sequence
aatcacttgaacccgggaggc

SEQ ID NO: 542 - DNA, 21 bp, Artificial Sequence
  Knock-Down Sequence
aagtgattctcatgcctcagc

SEQ ID NO: 543 - DNA, 21 bp, Artificial Sequence
  Knock-Down Sequence
aatcccagctactcaggaggc

SEQ ID NO: 544 - DNA, 21 bp, Artificial Sequence
  Knock-Down Sequence
aatcccagctactcaggaggc

SEQ ID NO: 545 - DNA, 21 bp, Artificial Sequence
  Knock-Down Sequence
aaagtagctgggattacaggc

SEQ ID NO: 546 - DNA, 21 bp, Artificial Sequence
  Knock-Down Sequence
acagagtctcgctattgtcac

SEQ ID NO: 547 - DNA, 21 bp, Artificial Sequence
  Knock-Down Sequence
acctgggttccagcaattctc

SEQ ID NO: 548 - DNA, 21 bp, Artificial Sequence
  Knock-Down Sequence
aagcaattctcctgcctcagc

SEQ ID NO: 549 - DNA, 21 bp, Artificial Sequence
  Knock-Down Sequence
aactcctgaccttaggtgatc

SEQ ID NO: 550 - DNA, 21 bp, Artificial Sequence
  Knock-Down Sequence
actcctgaccttaggtgatcc

SEQ ID NO: 551 - DNA, 21 bp, Artificial Sequence
  Knock-Down Sequence
tcacgcctgtaatcccagcac

SEQ ID NO: 552 - DNA, 21 bp, Artificial Sequence
  Knock-Down Sequence
actgggattacaggcgtgagc

SEQ ID NO: 553 - DNA, 21 bp, Artificial Sequence
  Knock-Down Sequence
acggtgaaaccctgtctctac

SEQ ID NO: 554 - DNA, 21 bp, Artificial Sequence
  Knock-Down Sequence
aacatggtgaaaccctgtctc

SEQ ID NO: 555 - DNA, 21 bp, Artificial Sequence
  Knock-Down Sequence
aacatggtgaaaccctgtctc

SEQ ID NO: 556 - DNA, 21 bp, Artificial Sequence
  Knock-Down Sequence
acagggtttcaccatgttggc

SEQ ID NO: 557 - DNA, 21 bp, Artificial Sequence
  Knock-Down Sequence
cctggccaacatggtgaaacc

SEQ ID NO: 558 - DNA, 21 bp, Artificial Sequence
  Knock-Down Sequence
gcctggccaacatggtgaaac

SEQ ID NO: 559 - DNA, 21 bp, Artificial Sequence
  Knock-Down Sequence
aactcctgacctcaggtaatc

SEQ ID NO: 560 - DNA, 21 bp, Artificial Sequence
  Knock-Down Sequence
tcacacctgtaatcccagcac

SEQ ID NO: 561 - DNA, 21 bp, Artificial Sequence
  Knock-Down Sequence
actcacacctgtaatcccagc

SEQ ID NO: 562 - DNA, 21 bp, Artificial Sequence
  Knock-Down Sequence
gctcacacctgtaatcccagc

SEQ ID NO: 563 - DNA, 19 bp, Artificial Sequence
  Knock-Down Sequence
tagagctggagcaggactc

SEQ ID NO: 564 - DNA, 19 bp, Artificial Sequence
  Knock-Down Sequence
ctacctgactggcaccatc

SEQ ID NO: 565 - DNA, 19 bp, Artificial Sequence
  Knock-Down Sequence
atcctacgccggaagagtc

SEQ ID NO: 566 - DNA, 19 bp, Artificial Sequence
  Knock-Down Sequence
gagccaagcgctttgcttc

SEQ ID NO: 567 - DNA, 19 bp, Artificial Sequence
  Knock-Down Sequence
tgagtagatttgtggagac

SEQ ID NO: 568 - DNA, 19 bp, Artificial Sequence
  Knock-Down Sequence
actggtggtggcagatgac

SEQ ID NO: 569 - DNA, 19 bp, Artificial Sequence
  Knock-Down Sequence
cagtgatggcagcagcagc

SEQ ID NO: 570 - DNA, 19 bp, Artificial Sequence
  Knock-Down Sequence
ggccttcaagcacccaagc

SEQ ID NO: 571 - DNA, 19 bp, Artificial Sequence
  Knock-Down Sequence
cactttgacacagccattc

SEQ ID NO: 572 - DNA, 19 bp, Artificial Sequence
  Knock-Down Sequence
acagccattctgtttaccc

SEQ ID NO: 573 - DNA, 19 bp, Artificial Sequence
  Knock-Down Sequence
catgctccatgacaactcc

SEQ ID NO: 574 - DNA, 19 bp, Artificial Sequence
  Knock-Down Sequence
tgacttcctggacaatggc

SEQ ID NO: 575 - DNA, 19 bp, Artificial Sequence
  Knock-Down Sequence
aatggctatgggcactgtc

SEQ ID NO: 576 - DNA, 19 bp, Artificial Sequence
  Knock-Down Sequence
tggctatgggcactgtctc

SEQ ID NO: 577 - DNA, 19 bp, Artificial Sequence
  Knock-Down Sequence
aaaccagaggctccattgc

SEQ ID NO: 578 - DNA, 19 bp, Artificial Sequence
  Knock-Down Sequence
ccagaggctccattgcatc

SEQ ID NO: 579 - DNA, 19 bp, Artificial Sequence
  Knock-Down Sequence
ggactatgatgctgaccgc

SEQ ID NO: 580 - DNA, 19 bp, Artificial Sequence
  Knock-Down Sequence
tcacgccattgtccacagc

SEQ ID NO: 581 - DNA, 19 bp, Artificial Sequence
  Knock-Down Sequence
tattccacaggctggtggc

SEQ ID NO: 582 - DNA, 19 bp, Artificial Sequence
  Knock-Down Sequence
cgacctcttcaagagcttc

SEQ ID NO: 583 - DNA, 19 bp, Artificial Sequence
  Knock-Down Sequence
cagtgcaaactcacctgcc

SEQ ID NO: 584 - DNA, 19 bp, Artificial Sequence
  Knock-Down Sequence
tactatgtgctggagccac

SEQ ID NO: 585 - DNA, 19 bp, Artificial Sequence
  Knock-Down Sequence
ggttctggttgcagcaagc

SEQ ID NO: 586 - DNA, 19 bp, Artificial Sequence
  Knock-Down Sequence
aacaatgtggtcactatcc

SEQ ID NO: 587 - DNA, 19 bp, Artificial Sequence
  Knock-Down Sequence
tacacgctgatgccctccc

SEQ ID NO: 588 - DNA, 19 bp, Artificial Sequence
  Knock-Down Sequence
gtcctagtggctggcaacc

SEQ ID NO: 589 - DNA, 19 bp, Artificial Sequence
  Knock-Down Sequence
acgcctccgatacagcttc

SEQ ID NO: 590 - DNA, 19 bp, Artificial Sequence
  Knock-Down Sequence
ataacctcactatcccggc

SEQ ID NO: 591 - DNA, 19 bp, Artificial Sequence
  Knock-Down Sequence
agccctccatctaaactgc

SEQ ID NO: 592 - DNA, 19 bp, Artificial Sequence
  Knock-Down Sequence
aacctgttctgctttcctc

SEQ ID NO: 593 - DNA, 19 bp, Artificial Sequence
  Knock-Down Sequence
ctgttctgctttcctcttc

SEQ ID NO: 594 - DNA, 19 bp, Artificial Sequence
  Knock-Down Sequence
agtcaagggtagggtgggc

SEQ ID NO: 595 - DNA, 19 bp, Artificial Sequence
  Knock-Down Sequence
agaatctcgctctgtcgcc

SEQ ID NO: 596 - DNA, 19 bp, Artificial Sequence
  Knock-Down Sequence
tggcacaatctcggctcac

SEQ ID NO: 597 - DNA, 19 bp, Artificial Sequence
  Knock-Down Sequence
aatctcggctcactgcatc

SEQ ID NO: 598 - DNA, 19 bp, Artificial Sequence
  Knock-Down Sequence
tcacttgaacccgggaggc

SEQ ID NO: 599 - DNA, 19 bp, Artificial Sequence
  Knock-Down Sequence
gtgattctcatgcctcagc

SEQ ID NO: 600 - DNA, 19 bp, Artificial Sequence
  Knock-Down Sequence
tcccagctactcaggaggc

SEQ ID NO: 601 - DNA, 19 bp, Artificial Sequence
  Knock-Down Sequence
tcccagctactcaggaggc

SEQ ID NO: 602 - DNA, 19 bp, Artificial Sequence
  Knock-Down Sequence
agtagctgggattacaggc

SEQ ID NO: 603 - DNA, 19 bp, Artificial Sequence
  Knock-Down Sequence
agagtctcgctattgtcac

SEQ ID NO: 604 - DNA, 19 bp, Artificial Sequence
  Knock-Down Sequence
ctgggttccagcaattctc

SEQ ID NO: 605 - DNA, 19 bp, Artificial Sequence
  Knock-Down Sequence
gcaattctcctgcctcagc

SEQ ID NO: 606 - DNA, 19 bp, Artificial Sequence
  Knock-Down Sequence
ctcctgaccttaggtgatc

SEQ ID NO: 607 - DNA, 19 bp, Artificial Sequence
  Knock-Down Sequence
tcctgaccttaggtgatcc

SEQ ID NO: 608 - DNA, 19 bp, Artificial Sequence
  Knock-Down Sequence
acgcctgtaatcccagcac

SEQ ID NO: 609 - DNA, 19 bp, Artificial Sequence
  Knock-Down Sequence
tgggattacaggcgtgagc

SEQ ID NO: 610 - DNA, 19 bp, Artificial Sequence
  Knock-Down Sequence
ggtgaaaccctgtctctac

SEQ ID NO: 611 - DNA, 19 bp, Artificial Sequence
  Knock-Down Sequence
catggtgaaaccctgtctc

SEQ ID NO: 612 - DNA, 19 bp, Artificial Sequence
  Knock-Down Sequence
catggtgaaaccctgtctc

SEQ ID NO: 613 - DNA, 19 bp, Artificial Sequence
  Knock-Down Sequence
agggtttcaccatgttggc

SEQ ID NO: 614 - DNA, 19 bp, Artificial Sequence
  Knock-Down Sequence
tggccaacatggtgaaacc

SEQ ID NO: 615 - DNA, 19 bp, Artificial Sequence
  Knock-Down Sequence
ctggccaacatggtgaaac

SEQ ID NO: 616 - DNA, 19 bp, Artificial Sequence
  Knock-Down Sequence
ctcctgacctcaggtaatc

SEQ ID NO: 617 - DNA, 19 bp, Artificial Sequence
  Knock-Down Sequence
acacctgtaatcccagcac

SEQ ID NO: 618 - DNA, 19 bp, Artificial Sequence
  Knock-Down Sequence
tcacacctgtaatcccagc

SEQ ID NO: 619 - DNA, 19 bp, Artificial Sequence
  Knock-Down Sequence
gctcacacctgtaatccca

(19) United States
(12) Patent Application Publication
(10) Pub. No.: US 2006/0281699 A1
(43) Pub. Date: Dec. 14, 2006

(54) Methods, compositions and compound assays for inhibiting amyloid-beta protein production

(76) Inventors: Pascal Gerard Merchiers, Tielen (BE); Marcel Hoffmann, Uithoorn (NL); Koenraad Frederik Florentina Spittaels, Puurs (BE); Wendy Laenen, Lier (BE)

Correspondence Address: SYNNESTVEDT & LECHNER, LLP, 2600 ARAMARK TOWER, 1101 MARKET STREET, PHILADELPHIA, PA, 191072950, US

(21) Appl. No.: 11/127,581

(22) Filed: May 12, 2005

Related U.S. Application Data
(60) Provisional application No. 60/570,352, filed on May 12, 2004.
(60) Provisional application No. 60/603,948, filed on Aug. 24, 2004.

(51) Int. Cl. A61K 48/00; A61K 31/165; C40B 30/06; C40B 40/08

(52) U.S. Cl. 514/44; 435/5; 435/7.1; 514/616

(57) ABSTRACT

A method for identifying compounds that inhibit amyloid-beta precursor protein processing in cells, comprising contacting a test compound with a PROTEASE polypeptide, or fragment thereof, and measuring a compound-PROTEASE property related to the production of amyloid-beta peptide. Cellular assays of the method measure indicators including cleaved protease substrate and/or amyloid beta peptide levels. Therapeutic methods, and pharmaceutical compositions including effective amyloid-beta precursor processing-inhibiting amounts of PROTEASE expression inhibitors, are useful for treating conditions involving cognitive impairment such as Alzheimer's disease.

### CROSS-REFERENCE TO RELATED APPLICATIONS

This application claims priority to U.S. Provisional Application No. 60/570,352, filed May 12, 2004, and U.S. Provisional Application No. 60/603,948, filed Aug. 24, 2004, the disclosures of which are incorporated herein by reference.

### FIELD OF THE INVENTION

This invention relates to the field of mammalian neuronal cell disorders, and in particular, to methods for identifying effective compounds, and therapies and compositions using such compounds, useful for the prevention and treatment of diseases associated with progressive loss of intellectual capacities in humans.

The neurological disorder that is most widely known for its progressive loss of intellectual capacities is Alzheimer's disease (AD). Worldwide, about 20 million people suffer from Alzheimer's disease. AD is clinically characterized by the initial loss of memory, followed by disorientation, impairment of judgment and reasoning, which is commonly referred to as cognitive impairment, and ultimately by full dementia. AD patients finally lapse into a severely debilitated, immobile state between four and twelve years after onset of the disease.

The key pathological evidence for AD is the presence of extracellular amyloid plaques and intracellular tau tangles in the brain, which are associated with neuronal degeneration (Ritchie and Lovestone (2002)). The extracellular amyloid plaques are believed to result from an increase in the insoluble amyloid beta peptide 1-42 produced by the metabolism of amyloid-beta precursor protein (APP). Following secretion, these amyloid beta 1-42 peptides form amyloid fibrils more readily than the amyloid beta 1-40 peptides, which are predominantly produced in healthy people. It appears that the amyloid beta peptide is on top of the neurotoxic cascade: experiments show that amyloid beta fibrils, when injected into the brains of P301L tau transgenic mice, enhance the formation of neurofibrillary tangles (Gotz et al. (2001)). In fact, a variety of amyloid beta peptides have been identified as amyloid beta peptides 1-42, 1-40, 1-39, 1-38, 1-37, which can be found in plaques and are often seen in cerebral spinal fluid.

The amyloid beta peptides are generated (or processed) from the membrane anchored APP, after cleavage by beta secretase and gamma secretase at position 1 and 40 or 42, respectively (FIG. 1A) (Annaert and De Strooper (2002)). In addition, high activity of beta secretase results in a shift of the cleavage at position 1 to position 11. Cleavage of amyloid-beta precursor protein by alpha secretase activity at position 17 and gamma secretase activity at 40 or 42 generates the non-pathological p3 peptide. Beta secretase is identified as the membrane anchored aspartyl protease BACE, while gamma secretase is a protein complex comprising presenilin 1 (PS1) or presenilin 2 (PS2), nicastrin, Anterior Pharynx Defective 1 (APH1) and Presenilin Enhancer 2 (PEN2). Of these proteins, the presenilins are widely thought to constitute the catalytic activity of the gamma secretase, while the other components play a role in the maturation and localization of the complex. The identity of the alpha secretase is still illustrious, although some results point towards the proteases ADAM 10 and TACE, which could have redundant functions.

A small fraction of AD cases (mostly early onset AD) are caused by autosomal dominant mutations in the genes encoding presenilin 1 and 2 (PS1; PS2) and the amyloid-beta precursor protein (APP), and it has been shown that mutations in APP, PS1 and PS2 alter the metabolism of amyloid-beta precursor protein leading to such increased levels of amyloid beta 1-42 produced in the brain. Although no mutations in PS1, PS2 and amyloid-beta precursor protein have been identified in late onset AD patients, the pathological characteristics are highly similar to the early onset AD patients. These increased levels of amyloid beta peptide could originate progressively with age from disturbed amyloid-beta precursor protein processing (e.g. high cholesterol levels enhance amyloid beta peptide production) or from decreased amyloid beta peptide catabolism. Therefore, it is generally accepted that AD in late onset AD patients is also caused by aberrant increased amyloid peptide levels in the brains. The level of these amyloid beta peptides, and more particularly amyloid-beta peptide 1-42, is increased in Alzheimer patients compared to the levels of these peptides in healthy persons. Thus, reducing the levels of these amyloid beta peptides is likely to be beneficial for patients with cognitive impairment.

### Reported Developments

The major current AD therapies are limited to delaying progressive memory loss by inhibiting the acetylcholinesterase enzyme, which increases acetylcholine neurotransmitter levels, which fall because the cholinergic neurons are the first neurons to degenerate during AD. This therapy does not halt the progression of the disease.

Therapies aimed at decreasing the levels of amyloid beta peptides in the brain, are increasingly being investigated and focus on the perturbed amyloid-beta precursor protein processing involving the beta- or gamma secretase enzymes.

The present invention is based on the discovery that certain known polypeptides are factors in the up-regulation and/or induction of amyloid beta precursor processing in neuronal cells, and that the inhibition of the function of such polypeptides are effective in reducing levels of amyloid beta peptides.

### SUMMARY OF THE INVENTION

The present invention relates to the relationship between the function of selected proteases (“PROTEASES”) and amyloid-beta precursor protein processing in mammalian cells.

One aspect of the present invention is a method for identifying a compound that inhibits the processing of amyloid-beta precursor protein in a mammalian cell, comprising
(a) contacting a compound with a polypeptide comprising an amino acid sequence selected from the group consisting of SEQ ID NO: 7, 8, 9, and 10; and
(b) measuring a compound-polypeptide property related to the production of amyloid-beta peptide.

Aspects of the present method include the in vitro assay of compounds using polypeptide of a PROTEASE, and cellular assays wherein PROTEASE inhibition is followed by observing indicators of efficacy, including cleaved protease substrate levels and/or amyloid beta peptide levels.

Another aspect of the invention is a method of treatment or prevention of a condition involving cognitive impairment, or a susceptibility to the condition, in a subject suffering or susceptible thereto, by administering a pharmaceutical composition comprising an effective amyloid-beta precursor processing-inhibiting amount of a PROTEASE inhibitor.

A further aspect of the present invention is a pharmaceutical composition for use in said method wherein said inhibitor comprises a polynucleotide selected from the group of an antisense polynucleotide, a ribozyme, and a small interfering RNA (siRNA), wherein said agent comprises a nucleic acid sequence complementary to, or engineered from, a naturally occurring polynucleotide sequence encoding a polypeptide, comprising an amino acid sequence selected from the group consisting of SEQ ID NO: 7, 8, 9, and 10, or a fragment thereof,

Another further aspect of the present invention is a pharmaceutical composition comprising a therapeutically effective amyloid-beta precursor processing-inhibiting amount of a PROTEASE inhibitor or its pharmaceutically acceptable salt, hydrate, solvate, or prodrug thereof in admixture with a pharmaceutically acceptable carrier. The present polynucleotides and PROTEASE inhibitor compounds are also useful for the manufacturing of a medicament for the treatment of Alzheimer's disease.

### BRIEF DESCRIPTION OF THE DRAWINGS

FIG. 1A: APP processing: The membrane anchored amyloid precursor protein (APP) is processed by two pathways: the amyloidogenic and non amyloidogenic pathway. In the latter pathway, APP is cleaved first by alpha secretase and then by gamma secretase, yielding the p3 peptides (17-40 or 17-42). The amyloidogenic pathway generates the pathogenic amyloid beta peptides (A beta) after cleavage by beta- and gamma-secretase respectively. The numbers depicted are the positions of the amino acids comprising the A beta sequences.

FIG. 2: Evaluation of the APP processing assay: Positive (PS1G384L; PS1L392V and BACE1) and negative (eGFP, LacZ and empty) control viruses are infected in Hek293APPwt at random MOI, mimicking a screening. A and B: Transduction is performed respectively with 1 and 0.2 μl of virus and amyloid beta 1-42 levels are performed. Data are represented as relative light units and correlate to pM of amyloid beta 1-42.

FIG. 3: Positive (PS1G384L and BACE1) and negative (eGFP, LacZ and empty) control viruses are infected in Hek293APPwt at random MOI. Transduction is performed respectively with 0.2 μl of virus and amyloid beta 1-42 levels are determined. Data are represented as single relative light units data points. The average and standard deviation of all negative controls is calculated and the cut off is determined using the AVERAGE+(3*STDEV) formula. The cut off is depicted as a line. All positive controls are clearly positioned above the cut-off.

FIGS. 4-7. Modulation of amyloid beta peptide levels by over-expression of the identified targets: USP21 [FIG. 4], GZMM [FIG. 5A-5B], USP2 [FIG. 6A-6B], ADAMTS4 [FIG. 7A-7B], in Hek293 APPwt cells: Hek293 APPwt cells were transduced with increasing MOI of empty adenovirus and adenoviruses harbouring cDNA's expressing the targets as indicated. Amyloid beta (Abeta) peptide levels were monitored through the amyloid beta 1-42, amyloid beta 1-40, amyloid beta 1-x and amyloid beta x-42 ELISAs, as indicated.

FIG. 8. Transfection with siRNA targeting USP21 reduces amyloid beta 1-42 levels. HEK293 APPwt c129 cells were transfected with the siRNAs targeted against eGFP, Luciferase, BACE and USP21 (A) or GZMM (B) as representatives of the targets disclosed herein, and 24 hours after transfection, medium was refreshed and cells were allowed to accumulate amyloid beta for 24 hours (48 hours post transfection). Amyloid beta (Abeta) was determined using the amyloid beta 1-42 ELISA as described intra. Data are presented in pM of amyloid beta. Cell viability was determined measuring ATP levels (ATP Glow kit, Promega, US). Amyloid beta 1-42 levels were normalized for ATP levels.

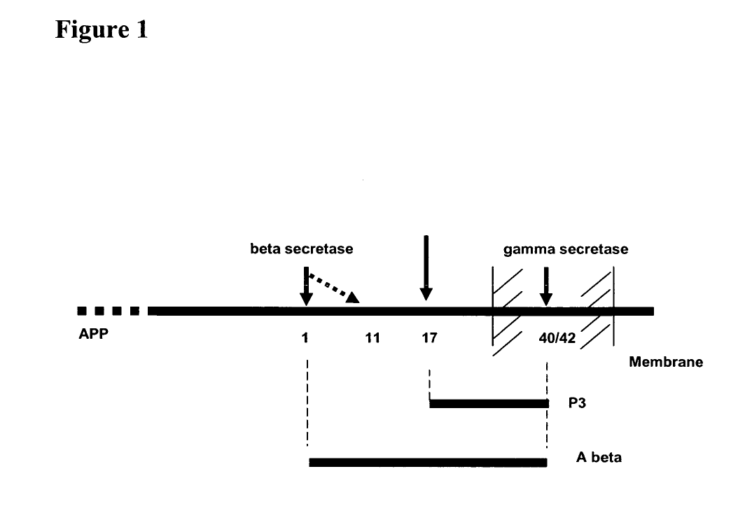

### DETAILED DESCRIPTION

The following terms are intended to have the meanings presented therewith below and are useful in understanding the description of and intended scope of the present invention.

Definitions:

The term “amyloid beta peptide” means amyloid beta peptides processed from the amyloid beta precursor protein (APP). The most common peptides include amyloid beta peptides 1-40, 1-42, 11-40 and 11-42. Other less prevalent amyloid beta peptide species are included in the subgenus of amyloid beta peptides described as x-42, whereby x ranges from 2-17, and 1-y whereby y ranges from 24-39 and 41. For descriptive and technical purposes hereinbelow, “x” has a value of 2-17, and “y” has a value of 24 to 41.

The term “carrier” means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as TWEEN™, polyethylene glycol (PEG), and PLURONICS™.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “contact” or “contacting” means bringing at least two moieties together, whether in an in vitro system or an in vivo system.

The term “condition” or “disease” means the overt presentation of symptoms (i.e., illness) or the manifestation of abnormal clinical indicators (e.g., biochemical indicators), resulting from defects in one amyloid beta protein precursor processing. Alternatively, the term “disease” refers to a genetic or environmental risk of or propensity for developing such symptoms or abnormal clinical indicators.

The term “endogenous” shall mean a material that a mammal naturally produces. Endogenous in reference to the term “protease” shall mean that which is naturally produced by a mammal (for example, and not limitation, a human). In contrast, the term non-endogenous in this context shall mean that which is not naturally produced by a mammal (for example, and not limitation, a human). Both terms can be utilized to describe both “in vivo” and “in vitro” systems. For example, and not a limitation, in a screening approach, the endogenous or non-endogenous protease may be in reference to an in vitro screening system. As a further example and not limitation, where the genome of a mammal has been manipulated to include a non-endogenous protease, screening of a candidate compound by means of an in vivo system is viable.

The term “expression” comprises both endogenous expression and overexpression by transduction.

The term “expressible nucleic acid” means a nucleic acid coding for a proteinaceous molecule, an RNA molecule, or a DNA molecule.

The term “hybridization” means any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (e.g., C₀ₜ or R₀ₜ analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed). The term “stringent conditions” refers to conditions that permit hybridization between polynucleotides and the claimed polynucleotides. Stringent conditions can be defined by salt concentration, the concentration of organic solvent, e.g., formamide, temperature, and other conditions well known in the art. In particular, reducing the concentration of salt, increasing the concentration of formamide, or raising the hybridization temperature can increase stringency.

The term “inhibit” or “inhibiting”, in relationship to the term “response” means that a response is decreased or prevented in the presence of a compound as opposed to in the absence of the compound.

The term “PROTEASE” or “PROTEASES” means the protein proteases identified in accordance with the present amyloid peptide assay to be involved in the induction of amyloid beta peptide levels. The preferred PROTEASES are identified in Table 5. The most preferred PROTEASES are the protein proteases, ubiquitin specific protease 21 (USP21), granzyme M (GZMM), ubiquitin specific protease 2 (USP2), and a disintegrin-like and metalloprotease (reprolysin type) with thrombospondin type 1 motif, 4 (ADAMTS4).

The term “ligand” means an endogenous, naturally occurring molecule specific for an endogenous, naturally occurring receptor.

The term “pharmaceutically acceptable prodrugs” as used herein means the prodrugs of the compounds useful in the present invention, which are, within the scope of sound medical judgment, suitable for use in contact with the tissues of patients with undue toxicity, irritation, allergic response commensurate with a reasonable benefit/risk ratio, and effective for their intended use of the compounds of the invention. The term “prodrug” means a compound that is transformed in vivo to yield an effective compound useful in the present invention or a pharmaceutically acceptable salt, hydrate or solvate thereof. The transformation may occur by various mechanisms, such as through hydrolysis in blood. The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as pro-drugs. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, 1-38, (1992); J. Pharm. Sci., 77,285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference. An example of the prodrugs is an ester prodrug. “Ester prodrug” means a compound that is convertible in vivo by metabolic means (e.g., by hydrolysis) to an inhibitor compound according to the present invention. For example an ester prodrug of a compound containing a carboxy group may be convertible by hydrolysis in vivo to the corresponding carboxy group.

The term “pharmaceutically acceptable salts” refers to the non-toxic, inorganic and organic acid addition salts, and base addition salts, of compounds of the present invention. These salts can be prepared in situ during the final isolation and purification of compounds useful in the present invention.

The term “polynucleotide” means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more preferably 70 percent of its base pairs are in common, most preferably 90 percent, and in a special embodiment 100 percent of its base pairs. The polynucleotides include polyribonucleic acids, polydeoxyribonucleic acids, and synthetic analogues thereof. The polynucleotides are described by sequences that vary in length, that range from about 10 to about 5000 bases, preferably about 100 to about 4000 bases, more preferably about 250 to about 2500 bases. A preferred polynucleotide embodiment comprises from about 10 to about 30 bases in length. A special embodiment of polynucleotide is the polyribonucleotide of from about 10 to about 22 nucleotides, more commonly described as small interfering RNAs (siRNAs). Another special embodiment are nucleic acids with modified backbones such as peptide nucleic acid (PNA), polysiloxane, and 2′-O-(2-methoxy)ethylphosphorothioate, or including non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection.

The term “polypeptide” relates to proteins (such as PROTEASES), proteinaceous molecules, fractions of proteins peptides and oligopeptides.

The term “solvate” means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term “subject” includes humans and other mammals.

The term “effective amount” or “therapeutically effective amount” means that amount of a compound or agent that will elicit the biological or medical response of a subject that is being sought by a medical doctor or other clinician. In particular, with regard to treating an neuronal disorder, the term “effective amount” is intended to mean that effective amyloid-beta precursor processing inhibiting amount of an compound or agent that will bring about a biologically meaningful decrease in the levels of amyloid beta peptide in the subject's brain tissue.

The term “treating” means an intervention performed with the intention of preventing the development or altering the pathology of, and thereby alleviating a disorder, disease or condition, including one or more symptoms of such disorder or condition. Accordingly, “treating” refers to both therapeutic treatment and prophylactic or preventative measures. Those in need of treating include those already with the disorder as well as those in which the disorder is to be prevented. The related term “treatment,” as used herein, refers to the act of treating a disorder, symptom, disease or condition, as the term “treating” is defined above.

The background of the present inventors' discovery is described briefly below.

Background of the PROTEASES

Ubiquitin, a highly conserved protein involved in the regulation of intracellular protein breakdown, cell cycle regulation, and stress response, is released from degraded proteins by disassembly of the polyubiquitin chains. The disassembly process is mediated by ubiquitin-specific proteases (USPs). SEQ ID NO: 1 (ubiquitin specific protease 21) and SEQ ID NO: 3 (ubiquitin specific protease 2) encode ubiquitin-specific proteases (enzymes that remove ubiquitin from ubiquitinated proteins). The encoded proteins belong to the C19 peptidase family, also known as family 2 of ubiquitin carboxyl-terminal hydrolases. The peptidases of family C19 hydrolyse bonds involving the carboxyl group of the C-terminal Gly residue of ubiquitin. These ubiquitinyl bonds can be alpha-peptide bonds to the N-terminus of another ubiquitin molecule, or isopeptide bonds to the sidechain of Lys48 in another ubiquitin molecule or to the sidechain of a Lys residue in another protein. The varied specificities of peptidases in the family have been reviewed by Amerik & Hochstrasse (Ubiquitin-specific protease Doa4 (Saccharomyces cerevisiae). In Handbook of Proteolytic Enzymes, 2 edn (Barrett, A. J., Rawlings, N. D. & Woessner, J. F. eds), p. 1229-1231, Elsevier, London. 2004), Baker (Ubiquitin-specific proteases 4 and 15. In Handbook of Proteolytic Enzymes, 2 edn (Barrett, A. J., Rawlings, N. D. & Woessner, J. F. eds), p. 1232-1236, Elsevier, London 2004.), Everett (Ubiquitin-specific protease 7. In Handbook of Proteolytic Enzymes, 2 edn (Barrett, A. J., Rawlings, N. D. & Woessner, J. F. eds), p. 1236-1238, Elsevier, London 2004) and Wilkinson (Ubiquitin isopeptidase T. In Handbook of Proteolytic Enzymes, 2 edn (Barrett, A. J., Rawlings, N. D. & Woessner, J. F. eds), p. 1239-1243, Elsevier, London 2004). USP21 has been reported to be capable of removing NEDD8 from NEDD8 conjugates (Gong, L., T. Kamitani, S. Millas, and E. T. Yeh. 2000. Identification of a novel isopeptidase with dual specificity for ubiquitin- and NEDD8-conjugated proteins. J. Biol. Chem. 275:14212-14216.). USP21 has also been described as recognizing Ub as a substrate (Wada, H., K. Kito, L. S. Caskey, E. T. Yeh, and T. Kamitani. 1998. Cleavage of the C-terminus of NEDD8 by UCH-L3. Biochem. Biophys. Res. Commun. 251:688-692.). Alternatively spliced transcript variants encoding different isoforms have been identified.

A substrate for USPs is z-LRGG-MCA) (MCA=methylcoumaryl-7-amide, fluorophore). The peptide LRGG (SEQ ID NO: 69) mimics the carboxyterminus of ubiquitin which terminus is involved in isopeptidase formation. USPs cleave between the last glycine and the MCA (Mullally et al. 2001. Cyclopentenone prostaglandins of the J series inhibit the ubiquitin isopeptidase activity of the proteasome pathway. J Biol Chem 276: 30366-73).

Low potency inhibitors of USP21 and USP2 include the cyclopentone prostaglandins of the J series (Mullally et al. 2001. Cyclopentenone prostaglandins of the J series inhibit the ubiquitin isopeptidase activity of the proteasome pathway. J Biol Chem 276: 30366-73).

Human natural killer (NK) cells and activated lymphocytes express and store a distinct subset of neutral serine proteases together with proteoglycans and other immune effector molecules in large cytoplasmic granules. Serine proteases are released with perforin from the cytotoxic granules of NK cells and cytotoxic T lymphocytes. These serine proteases are collectively termed granzymes and include 4 distinct gene products: granzyme A, granzyme B, granzyme H, and Met-ase, also known as granzyme M. SEQ ID NO: 2 encodes granzyme M. Granzyme M has a unique Met-ase activity and is expressed almost exclusively in NK cells. In the presence of perforin, the protease activity of granzyme M rapidly and effectively induces target cell death. In contrast to other granzymes, cell death induced by granzyme M does not feature obvious DNA fragmentation, occurs independently of caspases, caspase activation, and perturbation of mitochondria. Granzyme M induced cell death is not inhibited by overexpression of Bcl-2 (Kelly, J. M., Waterhouse, N. J., Cretney, E., Browne, K. A., Ellis, S., Trapani, J. A. & Smyth, M. J. (2004) Granzyme M mediates a novel form of perforin-dependent cell death. J Biol Chem, 279(21), 22236-22242).

Substrates for GZMM include peptides comprising the motif XPDM/XPSM/XPAM/AAPM/ (SEQ ID NOS: 70, 71, 72, and 73, respectively) wherein X=any amino acid and cleavage occurs after the Methinine residue (Rukamp et al. 2004. Subsite specificities of granzyme M: a study of inhibitors and newly synthesized thiobenzyl ester substrates. Arch Biochem Biophys 422: 9-22).

ADAMTS4 (SEQ ID NO: 4), also named aggrecanase 1, encodes a disintegrin and metalloproteinase with thrombospondin motifs-4, and is a member of the ADAMTS protein family. Members of the family share several distinct protein modules, including a propeptide region, a metalloproteinase domain, a disintegrin-like domain, and a thrombospondin type 1 (TS) motif. Individual members of this family differ in the number of C-terminal TS motifs, and some have unique C-terminal domains. The enzyme encoded by this gene lacks a C-terminal TS motif, and is responsible for the degradation of aggrecan, a major proteoglycan aggregating proteoglycan of articular cartilage, and brevican, a brain-specific extracellular matrix protein. It is found also in aorta tissue, discs, tendons and in the perineuronal net.

ADAMTS4 hydrolyzes aggrecan at five different sites in vitro and in vivo (Tortorella, M. D. et al. (2000) J. Biol. Chem. 275, 18566-18573; Tortorella, M. D. et al. (2002) Matrix Biology 21, 499-511; Lohmander, L. S. et al. (1993) Arthritis Rheumat. 36, 1214-1222; and Malfait, A.-M. et al. (2002) J. Biol. Chem. 277, 22201-22208). Four cleavage sites are located in the chondroitin sulfate-rich region between aggrecan globular domains G2 and G3 (sites E1667-G1668, E1480-G1481, E1771-A1772, E1871-L1872), while one site is placed in the rodlike polypeptide between globular domains G1 and G2 (E373-A374). In addition to the aggrecan cleavage sites (the most important of which appears to be NITEGE/ARGSVI (SEQ ID NO: 74) corresponding to amino acids 368-379 of aggrecan), alpha 2 macroglobulin (between amino acids 690 and 691 (M/G)) and brevican (between amino acids 395 and 396 (E/S) are also substrates for cleavage.

Applicants' Invention Based on PROTEASE Relationship to Amyloid Beta Peptides

As noted above, the present invention is based on the present inventors' discovery that PROTEASES are factors in the up-regulation and/or induction of amyloid beta precursor processing in mammalian, and principally, neuronal cells, and that the inhibition of the function of such polypeptides is effective in reducing levels of amyloid beta protein peptides.

The present inventors are unaware of any prior knowledge linking PROTEASES, and more particularly USP21, GZMM, USP2, and ADAMTS4, with amyloid beta peptide formation and secretion. Table 1 below identifies the cDNA and protein sequences for USP21, GZMM, USP2, and ADAMTS4.

TABLE 1

| | | | SEQ ID NO: | |
|---|---|---|---|---|
| Accession | Description | Code | DNA | Protein |
| NM_012475 | ubiquitin specific | USP21 | 1 | 7 |
| | protease 21 | | | |
| NM_005317 | granzyme M | GZMM | 2 | 8 |
| NM_004205 | ubiquitin specific | USP2 | 3 | 9 |
| | protease 2 | | | |
| NM_005099 | a disintegrin-like and | ADAMTS4 | 4 | 10 |
| | metalloprotease | | | |
| | (reprolysin type) | | | |
| | with thrombospondin | | | |
| | type 1 motif, 4 | | | |

As discussed in more detail in the Experimental section below, the present inventors demonstrate that the knockdown of USP21, GZMM, USP2, and ADAMTS4 reduces amyloid beta 1-42 in the conditioned medium of transduced cells. The present invention is based on these findings and the recognition that the PROTEASES, and particularly, USP21, GZMM, USP2, and ADAMTS4, may be putative drug targets for Alzheimer's disease, in view of the expression of these proteins in brain tissue.

One aspect of the present invention is a method based on the aforesaid discovery for identifying a compound that inhibits the processing of amyloid-beta precursor protein in a mammalian cell, and may therefore be useful in reducing amyloid beta peptide levels in a subject. The present method comprises contacting a drug candidate compound with a PROTEASE polypeptide, or a fragment of said polypeptide, and measuring a compound-polypeptide property related to the production of amyloid-beta protein. The “compound-polypeptide property” is a measurable phenomenon chosen by the person of ordinary skill in the art, and based on the recognition that PROTEASE activation and deactivation is a causative factor in the activation and deactivation, respectively, of amyloid beta protein precursor processing, and an increase and decrease, respectively, of amyloid beta peptide levels. The measurable property may range from the binding affinity for a peptide domain of the PROTEASE polypeptide, to the level of any one of a number of cleaved protease substrate levels resulting from the activation or deactivation of the PROTEASE, to a reporter molecule property directly linked to the aforesaid cleaved substrate, and finally to the level of amyloid beta peptide secreted by the mammalian cell contacted with the compound.

Depending on the choice of the skilled artisan, the present assay method may be designed to function as a series of measurements, each of which is designed to determine whether the drug candidate compound is indeed acting on PROTEASE to thereby facilitate the amyloid beta peptide pathway. For example, an assay designed to determine the binding affinity of a compound to PROTEASE, or fragment thereof, may be necessary, but not sufficient, to ascertain whether the test compound would be useful for reducing amyloid beta peptide levels when administered to a subject. Nonetheless, such binding information would be useful in identifying a set of test compounds for use in an assay that would measure a different property, further down the biochemical pathway. Such second assay may be designed to confirm that the test compound, having binding affinity for a PROTEASE peptide, actually down-regulates or inhibits PROTEASE function in a mammalian cell. This further assay may measure a cleaved PROTEASE substrate that is a direct consequence of the activation or deactivation of the PROTEASE, or a synthetic reporter system responding thereto. Measuring a different cleaved protease substrate, and/or confirming that the assay system itself is not being affected directly in contrast to the PROTEASE pathway may further validate the assay. In this latter regard, suitable controls should always be in place to insure against false positive readings.

The order of taking these measurements is not believed to be critical to the practice of the present invention, which may be practiced in any order. For example, one may first perform a screening assay of a set of compounds for which no information is known respecting the compounds' binding affinity for PROTEASE. Alternatively, one may screen a set of compounds identified as having binding affinity for a PROTEASE peptide domain, or a class of compounds identified as being an inhibitor of a PROTEASE. However, for the present assay to be meaningful to the ultimate use of the drug candidate compounds, a measurement of the cleaved protease substrate(s), or the ultimate amyloid beta peptide levels, is necessary. Validation studies including controls, and measurements of binding affinity to PROTEASE are nonetheless useful in identifying a compound useful in any therapeutic or diagnostic application.

The present assay method may be practiced in vitro, using one or more of the PROTEASE proteins, or fragments thereof. The amino acid sequences of the preferred PROTEASES, USP21, GZMM, USP2, and ADAMTS4, are found in SEQ ID NO: 7, 8, 9, and 10. The binding affinity of the compound with the polypeptide can be measured by methods known in the art, such as using surface plasmon resonance biosensors (Biacore), by saturation binding analysis with a labeled compound (e.g. Scatchard and Lindmo analysis), by differential UV spectrophotometer, fluorescence polarization assay, Fluorometric Imaging Plate Reader (FLIPR®) system, Fluorescence resonance energy transfer, and Bioluminescence resonance energy transfer. The binding affinity of compounds can also be expressed in dissociation constant (Kd) or as IC50 or EC50. The IC50 represents the concentration of a compound that is required for 50% inhibition of binding of another ligand to the polypeptide. The EC50 represents the concentration required for obtaining 50% of the maximum effect in any assay that measures PROTEASE function. The dissociation constant, Kd, is a measure of how well a ligand binds to the polypeptide, it is equivalent to the ligand concentration required to saturate exactly half of the binding-sites on the polypeptide. Compounds with a high affinity binding have low Kd, IC50 and EC50 values, i.e. in the range of 100 nM to 1 pM; a moderate to low affinity binding relates to a high Kd, IC50 and EC50 values, i.e. in the micromolar range.

The present assay method may also be practiced in a cellular assay, A host cell expressing PROTEASE can be a cell with endogenous expression or a cell over-expressing the PROTEASE e.g. by transduction. When the endogenous expression of the polypeptide is not sufficient to determine a baseline that can easily be measured, one may use using host cells that over-express PROTEASE. Over-expression has the advantage that the level of the cleaved protease substrate is higher than the activity level by endogenous expression. Accordingly, measuring such levels using presently available techniques is easier. In such cellular assay, the biological activity of PROTEASE may be measured by following the production of a cleaved protease substrate. Cleaved protease substrate levels may be measured by several different techniques, either directly by ELISA or radioactive technologies. Increased presence of PROTEASE in a cell increases the level of secreted amyloid beta peptides.

The present invention further relates to a method for identifying a compound that inhibits amyloid-beta precursor protein processing in a mammalian cell comprising:
(a) contacting a compound with a polypeptide comprising an amino acid sequence selected from the group consisting of SEQ ID NO: 7, 8, 9, and 10,
(b) determining the binding affinity of the compound to the polypeptide,
(c) contacting a population of mammalian cells expressing said polypeptide with the compound that exhibits a binding affinity of at least 10 micromolar, and
(d) identifying the compound that inhibits the amyloid-beta precursor protein processing in the cells.

A further embodiment of the present invention relates a method to identify a compound that inhibits the amyloid-beta precursor protein processing in a cell, wherein the activity level of the PROTEASE polypeptide is measured by determining the level of amyloid beta peptides. The levels of these peptides may be measured with specific ELISAs using antibodies specifically recognizing the different amyloid beta peptide species (see e.g. EXAMPLE 1). Secretion of the various amyloid beta peptides may also be measured using antibodies that bind all peptides. Levels of amyloid beta peptides can also be measured by Mass spectrometry analysis.

For high-throughput purposes, libraries of compounds may be used such as antibody fragment libraries, peptide phage display libraries, peptide libraries (e.g. LOPAP™, Sigma Aldrich), lipid libraries (BioMol), synthetic compound libraries (e.g. LOPAC™, Sigma Aldrich) or natural compound libraries (Specs, TimTec).

Preferred drug candidate compounds are low molecular weight compounds. Low molecular weight compounds, i.e. with a molecular weight of 500 Dalton or less, are likely to have good absorption and permeation in biological systems and are consequently more likely to be successful drug candidates than compounds with a molecular weight above 500 Dalton (Lipinski et al. (1997)). Peptides comprise another preferred class of drug candidate compounds. Peptides may be excellent drug candidates and there are multiple examples of commercially valuable peptides such as fertility hormones and platelet aggregation inhibitors. Natural compounds are another preferred class of drug candidate compound. Such compounds are found in and extracted from natural sources, and which may thereafter be synthesized. The lipids are another preferred class of drug candidate compound.

Another preferred class of drug candidate compounds is an antibody. The present invention also provides antibodies directed against PROTEASE. These antibodies should be endogenously produced to bind to the intra-cellular PROTEASE domain. These antibodies may be monoclonal antibodies or polyclonal antibodies. The present invention includes chimeric, single chain, and humanized antibodies, as well as FAb fragments and the products of a FAb expression library, and Fv fragments and the products of an Fv expression library.

In certain embodiments, polyclonal antibodies may be used in the practice of the invention. The skilled artisan knows methods of preparing polyclonal antibodies. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. Antibodies may also be generated against the intact PROTEASE protein or polypeptide, or against a fragment, derivatives including conjugates, or other epitope of the PROTEASE protein or polypeptide, such as the PROTEASE embedded in a cellular membrane, or a library of antibody variable regions, such as a phage display library.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). One skilled in the art without undue experimentation may select the immunization protocol.

In some embodiments, the antibodies may be monoclonal antibodies. Monoclonal antibodies may be prepared using methods known in the art. The monoclonal antibodies of the present invention may be “humanized” to prevent the host from mounting an immune response to the antibodies. A “humanized antibody” is one in which the complementarity determining regions (CDRs) and/or other portions of the light and/or heavy variable domain framework are derived from a non-human immunoglobulin, but the remaining portions of the molecule are derived from one or more human immunoglobulins. Humanized antibodies also include antibodies characterized by a humanized heavy chain associated with a donor or acceptor unmodified light chain or a chimeric light chain, or vice versa. The humanization of antibodies may be accomplished by methods known in the art (see, e.g. Mark and Padlan, (1994) “Chapter 4. Humanization of Monoclonal Antibodies”, The Handbook of Experimental Pharmacology Vol. 113, Springer-Verlag, New York). Transgenic animals may be used to express humanized antibodies.

Human antibodies can also be produced using various techniques known in the art, including phage display libraries (Hoogenboom and Winter, (1991) J. Mol. Biol. 227:381-8; Marks et al. (1991). J. Mol. Biol. 222:581-97). The techniques of Cole, et al. and Boerner, et al. are also available for the preparation of human monoclonal antibodies (Cole, et al. (1985) Monoclonal Antibodies and Cancer Therapy, Alan R. Liss, p. 77; Boerner, et al (1991). J. Immunol., 147(1):86-95).

Techniques known in the art for the production of single chain antibodies can be adapted to produce single chain antibodies to the PROTEASE polypeptides and proteins of the present invention. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively; the relevant cysteine residues are substituted with another amino acid residue or are deleted so as to prevent cross-linking.

Bispecific antibodies are monoclonal, preferably human or humanized, antibodies that have binding specificities for at least two different antigens and preferably for a cell-surface protein or receptor or receptor subunit. In the present case, one of the binding specificities is for one domain of the PROTEASE; the other one is for another domain of the same or different PROTEASE.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, (1983) Nature 305:537-9). Because of the random assortment of immunoglobulin heavy and light chains, these hybridomas (quadromas) produce a potential mixture of ten different antibody molecules, of which only one has the correct bispecific structure. Affinity chromatography steps usually accomplish the purification of the correct molecule. Similar procedures are disclosed in Trauneeker, et al. (1991) EMBO J. 10:3655-9.

According to another preferred embodiment, the assay method uses a drug candidate compound identified as having a binding affinity for PROTEASES, and/or has already been identified as having down-regulating activity such as antagonist activity vis-à-vis one or more PROTEASE.

Methods to isolate compounds, and resulting compounds, that inhibit the activity of PROTEASES are for example, described in WO971827, WO9725437, WO9322429 and WO9851665 and U.S. Pat. No. 6,576,664 (referring to aggrecanase (ADAMTS4) inhibitors), hereby incorporated by reference.

Another aspect of the present invention relates to a method for reducing amyloid-beta precursor protein processing in a mammalian cell, comprising by contacting said cell with an expression-inhibiting agent that inhibits the translation in the cell of a polyribonucleotide encoding a PROTEASE polypeptide. A particular embodiment relates to a composition comprising a polynucleotide including at least one antisense strand that functions to pair the agent with the target PROTEASE mRNA, and thereby down-regulate or block the expression of PROTEASE polypeptide. The inhibitory agent preferably comprises antisense polynucleotide, a ribozyme, and a small interfering RNA (siRNA), wherein said agent comprises a nucleic acid sequence complementary to, or engineered from, a naturally-occurring polynucleotide sequence encoding a portion of a polypeptide comprising an amino acid sequence selected from the group consisting of SEQ ID NO: 7, 8, 9, and 10.

A special embodiment of the present invention relates to a method wherein the expression-inhibiting agent is selected from the group consisting of antisense RNA, antisense oligodeoxynucleotide (ODN), a ribozyme that cleaves the polyribonucleotide coding for SEQ ID NO: 7, 8, 9, and 10, a small interfering RNA (siRNA) that is sufficiently homologous to a portion of the polyribonucleotide corresponding to SEQ ID NO: 7, 8, 9, and 10 such that the siRNA interferes with the translation of the PROTEASE polyribonucleotide to the PROTEASE polypeptide.

Another embodiment of the present invention relates to a method wherein the expression-inhibiting agent is a nucleic acid expressing the antisense RNA, antisense oligodeoxynucleotide (ODN), a ribozyme that cleaves the polyribonucleotide coding for SEQ ID NO: 7, 8, 9, and 10, a small interfering RNA (siRNA) that is sufficiently homologous to a portion of the polyribonucleotide corresponding to SEQ ID NO: 7, 8, 9, and 10 such that the siRNA interferes with the translation of the PROTEASE polyribonucleotide to the PROTEASE polypeptide. Preferably the expression-inhibiting agent is an antisense RNA, ribozyme, antisense oligodeoxynucleotide, or siRNA comprising a nucleotide sequence selected from the group consisting of SEQ ID NO: 14-32, 49-68, and 332-876.

The down regulation of gene expression using antisense nucleic acids can be achieved at the translational or transcriptional level. Antisense nucleic acids of the invention are preferably nucleic acid fragments capable of specifically hybridizing with all or part of a nucleic acid encoding a PROTEASE polypeptide or the corresponding messenger RNA. In addition, antisense nucleic acids may be designed which decrease expression of the nucleic acid sequence capable of encoding a PROTEASE polypeptide by inhibiting splicing of its primary transcript. Any length of antisense sequence is suitable for practice of the invention so long as it is capable of down-regulating or blocking expression of a nucleic acid coding for a PROTEASE. Preferably, the antisense sequence is at least about 17 nucleotides in length. The preparation and use of antisense nucleic acids, DNA encoding antisense RNAs and the use of oligo and genetic antisense is known in the art.

One embodiment of expression-inhibitory agent is a nucleic acid that is antisense to a nucleic acid comprising SEQ ID NO: 1, 2, 3, and 4. For example, an antisense nucleic acid (e.g. DNA) may be introduced into cells in vitro, or administered to a subject in vivo, as gene therapy to inhibit cellular expression of nucleic acids comprising SEQ ID NO: 1, 2, 3, and 4. Antisense oligonucleotides preferably comprise a sequence containing from about 17 to about 100 nucleotides and more preferably the antisense oligonucleotides comprise from about 18 to about 30 nucleotides. Antisense nucleic acids may be prepared from about 10 to about 30 contiguous nucleotides selected from the sequences of SEQ ID NO: 1, 2, 3, and 4, expressed in the opposite orientation.

The antisense nucleic acids are preferably oligonucleotides and may consist entirely of deoxyribo-nucleotides, modified deoxyribonucleotides, or some combination of both. The antisense nucleic acids can be synthetic oligonucleotides. The oligonucleotides may be chemically modified, if desired, to improve stability and/or selectivity. Since oligonucleotides are susceptible to degradation by intracellular nucleases, the modifications can include, for example, the use of a sulfur group to replace the free oxygen of the phosphodiester bond. This modification is called a phosphorothioate linkage. Phosphorothioate antisense oligonucleotides are water soluble, polyanionic, and resistant to endogenous nucleases. In addition, when a phosphorothioate antisense oligonucleotide hybridizes to its target site, the RNA-DNA duplex activates the endogenous enzyme ribonuclease (RNase) H, which cleaves the mRNA component of the hybrid molecule.

In addition, antisense oligonucleotides with phosphoramidite and polyamide (peptide) linkages can be synthesized. These molecules should be very resistant to nuclease degradation. Furthermore, chemical groups can be added to the 2′ carbon of the sugar moiety and the 5 carbon (C-5) of pyrimidines to enhance stability and facilitate the binding of the antisense oligonucleotide to its target site. Modifications may include 2′-deoxy, O-pentoxy, O-propoxy, O-methoxy, fluoro, methoxyethoxy phosphorothioates, modified bases, as well as other modifications known to those of skill in the art.

Another type of expression-inhibitory agent that reduces the levels of PROTEASES is ribozymes. Ribozymes are catalytic RNA molecules (RNA enzymes) that have separate catalytic and substrate binding domains. The substrate binding sequence combines by nucleotide complementarity and, possibly, non-hydrogen bond interactions with its target sequence. The catalytic portion cleaves the target RNA at a specific site. The substrate domain of a ribozyme can be engineered to direct it to a specified mRNA sequence. The ribozyme recognizes and then binds a target mRNA through complementary base pairing. Once it is bound to the correct target site, the ribozyme acts enzymatically to cut the target mRNA. Cleavage of the mRNA by a ribozyme destroys its ability to direct synthesis of the corresponding polypeptide. Once the ribozyme has cleaved its target sequence, it is released and can repeatedly bind and cleave at other mRNAs.

Ribozyme forms include a hammerhead motif, a hairpin motif, a hepatitis delta virus, group I intron or RNaseP RNA (in association with an RNA guide sequence) motif or Neurospora VS RNA motif. Ribozymes possessing a hammerhead or hairpin structure are readily prepared since these catalytic RNA molecules can be expressed within cells from eukaryotic promoters (Chen, et al. (1992) Nucleic Acids Res. 20:4581-9). A ribozyme of the present invention can be expressed in eukaryotic cells from the appropriate DNA vector. If desired, the activity of the ribozyme may be augmented by its release from the primary transcript by a second ribozyme (Ventura, et al. (1993) Nucleic Acids Res. 21:3249-55).

Ribozymes may be chemically synthesized by combining an oligodeoxyribonucleotide with a ribozyme catalytic domain (20 nucleotides) flanked by sequences that hybridize to the target mRNA after transcription. The oligodeoxyribonucleotide is amplified by using the substrate binding sequences as primers. The amplification product is cloned into a eukaryotic expression vector.

Ribozymes are expressed from transcription units inserted into DNA, RNA, or viral vectors. Transcription of the ribozyme sequences are driven from a promoter for eukaryotic RNA polymerase I (pol (I), RNA polymerase II (pol II), or RNA polymerase III (pol III). Transcripts from pol II or pol III promoters will be expressed at high levels in all cells; the levels of a given pol II promoter in a given cell type will depend on nearby gene regulatory sequences. Prokaryotic RNA polymerase promoters are also used, providing that the prokaryotic RNA polymerase enzyme is expressed in the appropriate cells (Gao and Huang, (1993) Nucleic Acids Res. 21:2867-72). It has been demonstrated that ribozymes expressed from these promoters can function in mammalian cells (Kashani-Sabet, et al. (1992) Antisense Res. Dev. 2:3-15).

A particularly preferred inhibitory agent is a small interfering RNA (siRNA). siRNAs mediate the post-transcriptional process of gene silencing by double stranded RNA (dsRNA) that is homologous in sequence to the silenced RNA. siRNA according to the present invention comprises a sense strand of 17-25 nucleotides complementary or homologous to a contiguous 17-25 nucleotide sequence selected from the group of sequences described in SEQ ID NO: 1, 2, 3, and 4 and an antisense strand of 17-23 nucleotides complementary to the sense strand. Exemplary sequences are described as the KD sequences of SEQ ID NO: 14-32, 49-68, and 332-876. The most preferred siRNA comprises sense and anti-sense strands that are 100 percent complementary to each other and the target polynucleotide sequence. Preferably the siRNA further comprises a loop region linking the sense and the antisense strand.

A self-complementing single stranded siRNA molecule polynucleotide according to the present invention comprises a sense portion and an antisense portion connected by a loop region linker. Preferably, the loop region sequence is 4-30 nucleotides long, more preferably 5-15 nucleotides long and most preferably 8 nucleotides long. In a most preferred embodiment the linker sequence is UUGCUAUA (SEQ ID NO: 13). Self-complementary single stranded siRNAs form hairpin loops and are more stable than ordinary dsRNA. In addition, they are more easily produced from vectors.

Analogous to antisense RNA, the siRNA can be modified to confirm resistance to nucleolytic degradation, or to enhance activity, or to enhance cellular distribution, or to enhance cellular uptake, such modifications may consist of modified internucleoside linkages, modified nucleic acid bases, modified sugars and/or chemical linkage the SiRNA to one or more moieties or conjugates. The nucleotide sequences are selected according to siRNA designing rules that give an improved reduction of the target sequences compared to nucleotide sequences that do not comply with these siRNA designing rules (For a discussion of these rules and examples of the preparation of siRNA, WO2004094636, published Nov. 4, 2004, and UA20030198627, are hereby incorporated by reference).

The present invention also relates to compositions, and methods using said compositions, comprising a DNA expression vector capable of expressing a polynucleotide capable of inhibiting amyloid beta protein precursor processing and described hereinabove as an expression inhibition agent.

A special aspect of these compositions and methods relates to the down-regulation or blocking of the expression of a PROTEASE polypeptide by the induced expression of a polynucleotide encoding an intracellular binding protein that is capable of selectively interacting with the PROTEASE polypeptide. An intracellular binding protein includes any protein capable of selectively interacting, or binding, with the polypeptide in the cell in which it is expressed and neutralizing the function of the polypeptide. Preferably, the intracellular binding protein is a neutralizing antibody or a fragment of a neutralizing antibody having binding affinity to an epitope of the PROTEASE polypeptide of SEQ ID NO: 7, 8, 9, and 10. More preferably, the intracellular binding protein is a single chain antibody.

A special embodiment of this composition comprises the expression-inhibiting agent selected from the group consisting of antisense RNA, antisense oligodeoxynucleotide (ODN), a ribozyme that cleaves the polyribonucleotide coding for SEQ ID NO: 7, 8, 9, and 10, and a small interfering RNA (siRNA) that is sufficiently homologous to a portion of the polyribonucleotide corresponding to SEQ ID NO: 7, 8, 9, and 10 such that the siRNA interferes with the translation of the PROTEASE polyribonucleotide to the PROTEASE polypeptide,

The polynucleotide expressing the expression-inhibiting agent is preferably included within a vector. The polynucleic acid is operably linked to signals enabling expression of the nucleic acid sequence and is introduced into a cell utilizing, preferably, recombinant vector constructs, which will express the antisense nucleic acid once the vector is introduced into the cell. A variety of viral-based systems are available, including adenoviral, retroviral, adeno-associated viral, lentiviral, herpes simplex viral or a sendaviral vector systems, and all may be used to introduce and express polynucleotide sequence for the expression-inhibiting agents in target cells.

Preferably, the viral vectors used in the methods of the present invention are replication defective. Such replication defective vectors will usually pack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

In a preferred embodiment, the viral element is derived from an adenovirus. Preferably, the vehicle includes an adenoviral vector packaged into an adenoviral capsid, or a functional part, derivative, and/or analogue thereof. Adenovirus biology is also comparatively well known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses. Preferred adenoviral fiber protein sequences are serotype 17, 45 and 51. Techniques or construction and expression of these chimeric vectors are disclosed in US Published Patent Applications 20030180258 and 20040071660, hereby incorporated by reference.

In a preferred embodiment, the nucleic acid derived from an adenovirus includes the nucleic acid encoding an adenoviral late protein or a functional part, derivative, and/or analogue thereof. An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof. Preferably, the nucleic acid derived from an adenovirus includes the nucleic acid encoding adenovirus E2A or a functional part, derivative, and/or analogue thereof. Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell. The adenoviral vectors used in the examples of this application are exemplary of the vectors useful in the present method of treatment invention.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Lentiviral vector systems may also be used in the practice of the present invention. Retroviral systems and herpes virus system may be preferred vehicles for transfection of neuronal cells.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

In the vector construction, the polynucleotide agents of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda P.sub.r, P.sub.1, and trp promoters. Among the eukaryotic (including viral) promoters useful for practice of this invention are ubiquitous promoters (e.g. HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells, or Flt and Flk promoters active in endothelial cells), including animal transcriptional control regions, which exhibit tissue specificity and have been utilized in transgenic animals: elastase I gene control region which is active in pancreatic acinar cells (Swift, et al. (1984) Cell 38:639-46; Ornitz, et al. (1986) Cold Spring Harbor Symp. Quant. Biol. 50:399-409; MacDonald, (1987) Hepatology 7:425-515); insulin gene control region which is active in pancreatic beta cells (Hanahan, (1985) Nature 315:115-22), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), albumin gene control region which is active in liver (Pinkert, et al. (1987) Genes and Devel. 1:268-76), alpha-fetoprotein gene control region which is active in liver (Krumlauf, et al. (1985) Mol. Cell. Biol., 5:1639-48; Hammer, et al. (1987) Science 235:53-8), alpha 1-antitrypsin gene control region which is active in the liver (Kelsey, et al. (1987) Genes and Devel., 1: 161-71), beta-globin gene control region which is active in myeloid cells (Mogram, et al. (1985) Nature 315:338-40; Kollias, et al. (1986) Cell 46:89-94), myelin basic protein gene control region which is active in oligodendrocyte cells in the brain (Readhead, et al. (1987) Cell 48:703-12), myosin light chain-2 gene control region which is active in skeletal muscle (Sani, (1985) Nature 314.283-6), and gonadotropic releasing hormone gene control region which is active in the hypothalamus (Mason, et al. (1986) Science 234:1372-8).

Other promoters which may be used in the practice of the invention include promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1a, and MLP promoters.

Additional vector systems include the non-viral systems that facilitate introduction of polynucleotide agents into a patient. For example, a DNA vector encoding a desired sequence can be introduced in vivo by lipofection. Synthetic cationic lipids designed to limit the difficulties encountered with liposome-mediated transfection can be used to prepare liposomes for in vivo transfection of a gene encoding a marker (Felgner, et. al. (1987) Proc. Natl. Acad Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Felgner and Ringold, (1989) Nature 337:387-8). Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for therapeutic purposes can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

The present invention also provides biologically compatible compositions comprising the compounds identified as PROTEASE inhibitors, and the expression-inhibiting agents as described hereinabove.

A biologically compatible composition is a composition, that may be solid, liquid, gel, or other form, in which the compound, polynucleotide, vector, and antibody of the invention is maintained in an active form, e.g., in a form able to effect a biological activity. For example, a compound of the invention would have inverse agonist or antagonist activity on the PROTEASE; a nucleic acid would be able to replicate, translate a message, or hybridize to a complementary mRNA of a PROTEASE; a vector would be able to transfect a target cell and expression the antisense, antibody, ribozyme or siRNA as described hereinabove; an antibody would bind a PROTEASE polypeptide domain.

A preferred biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well-known non-toxic physiologically acceptable carriers, adjuvants and vehicles as desired.

A particularly preferred embodiment of the present composition invention is a cognitive-enhancing pharmaceutical composition comprising a therapeutically effective amount of an expression-inhibiting agent as described hereinabove, in admixture with a pharmaceutically acceptable carrier. Another preferred embodiment is a pharmaceutical composition for the treatment or prevention of a condition involving cognitive impairment or a susceptibility to the condition, comprising an effective amyloid beta peptide inhibiting amount of a PROTEASE antagonist or inverse agonist its pharmaceutically acceptable salts, hydrates, solvates, or prodrugs thereof in admixture with a pharmaceutically acceptable carrier. Particularly preferred compounds are disclosed in U.S. Pat. No. 6,576,664, and include the compounds including the N1-(2(R)-hydroxy-1(S)-indanyl)-N4-hydroxy-2(R)-substituted-butanediamide compounds having ADAMST4 inhibitory activity, and most preferably the following exemplary compounds.
N1-(2(R)-hydroxy-1(S)-indanyl)-N4-hydroxy-2(R)-isobutyl-butanediamide;
N1-(2(R)-hydroxy-1(S)-indanyl)-N4-hydroxy-2(R)-isobutyl-3(S)-(5-hydroxycarbonyl)-pentanamide;
N1-(2(R)-hydroxy-1(S)-indanyl)-N4-hydroxy-2(R)-isobutyl-3(S)-methyl-butanediamide;
N1-(2(R)-hydroxy-1(S)-indanyl)-N4-hydroxy-2(R)-isobutyl-3(S)-propyl-butanediamide;
N1-(2(R)-hydroxy-1(S)-indanyl)-N4-hydroxy-2(R)-hexyl-3(S)-propyl-butanediamide;
N1-[2(R)-hydroxy-1(S)-indanyl]-N4-hydroxy-2(R)-[(4-hydroxy-phenyl)methyl]butanediamide;
N1-[2(R)-hydroxy-1(S)-indanyl]-N4-hydroxy-2(R)-[(4-methoxy-phenyl)methyl]butanediamide;
N1-[1(S)-indanyl]-N4-hydroxy-2(R)-[(4-hydroxy-phenyl)methyl]butanediamide;
N1-[2(R)-hydroxy-1(S)-indanyl]-N4-hydroxy-2(R)-[3-phenyl-propyl]butanediamide;
N1-[2(R)-hydroxy-1(S)-indanyl]-N4-hydroxy-2(R)-[[4-(benzyloxy)-phenyl]methyl]butanediamide;
N1-[2(R)-hydroxy-1(S)-indanyl]-N4-hydroxy-2(R)-[[3-(benzyloxy)-phenyl]methyl]butanediamide;
N1-[2(R)-hydroxy-1(S)-indanyl]-N4-hydroxy-2(R)-[(3-hydroxy-phenyl)methyl]butanediamide;
N1-[2(R)-hydroxy-1(S)-indanyl]-N4-hydroxy-2(R)-[(4-fluoro-phenyl)methyl]butanediamide;
N1-[2(R)-hydroxy-1(S)-indanyl]-N4-hydroxy-2(R)-[(3,4-methylenedioxy-phenyl)methyl]butanediamide;
N1-[2(R)-hydroxy-1(S)-indanyl]-N4-hydroxy-2(R)-[(3-methoxy-phenyl)methyl]butanediamide;
N1-[2(R)-hydroxy-1(S)-indanyl]-N4-hydroxy-2(R)-[[4-(3-trifluoromethyl-phenyl)phenyl]methyl]butanediamide;
N1-[2(R)-hydroxy-1(S)-indanyl]-N4-hydroxy-2(R)-[[4-(2-tert-butylaminosulfonyl-phenyl)phenyl]methyl]-butanediamide;
N1-[2(R)-hydroxy-1(S)-indanyl]-N4-hydroxy-2(R)-[[4-(2-methoxy-phenyl)phenyl]methyl]butanediamide;
N1-[2(R)-hydroxy-1(S)-indanyl]-N4-hydroxy-2(R)-[[4-phenylphenyl]methyl]butanediamide;
N1-[2(R)-hydroxy-1(S)-indanyl]-N4-hydroxy-2(R)-[(3-hydroxy-4-methoxy-phenyl)methyl]butanediamide;
N1-[2(R)-hydroxy-1(S)-indanyl]-N4-hydroxy-2(R)-[[4-(2-chloro-phenyl)phenyl]methyl]butanediamide;
N1-[2(R)-hydroxy-1(S)-indanyl]-N4-hydroxy-2(R)-[[4-(benzofuran-2-yl)phenyl]methyl]butanediamide;
N1-[2(R)-hydroxy-1(S)-indanyl]-N4-hydroxy-2(R)-[[4-(2-methyl-phenyl)phenyl]methyl]butanediamide;
N1-[2(R)-hydroxy-1(S)-indanyl]-N4-hydroxy-2(R)-[[(3,4-methylenedioxy-phenyl)phenyl]methyl]butanediamide;
N1-[2(R)-hydroxy-1(S)-indanyl]-N4-hydroxy-2(R)-[[4-((tetrazol-2-yl-phenyl)phenyl]methyl]butanediamide;
N1-[2(R)-hydroxy-1(S)-indanyl]-N4-hydroxy-2(R)-[[3-phenylphenyl]methyl]butanediamide;
N1-[2(R)-hydroxy-1(S)-indanyl]-N4-hydroxy-2(R)-[[(3-methyl-phenyl)phenyl]methyl]butanediamide;
N1-[2(R)-hydroxy-1(S)-indanyl]-N4-hydroxy-2(R)-[(4-amino-phenyl)methyl]butanediamide;
N1-[2(R)-hydroxy-1(S)-indanyl]-N4-hydroxy-2(R)-[[((4-benzyloxy-carbonyl)amino)phenyl]methyl]butanediamide;
N1-[2(R)-hydroxy-1(S)-indanyl]-N4-hydroxy-2(R)-[[4-(2-hydroxymethylphenyl)phenyl]methyl]butanediamide;
N1-[2(R)-hydroxy-1(S)-indanyl]-N4-hydroxy-2(R)-[[4-(3,4,5-trimethoxy-phenyl)phenyl]methyl]butanediamide;
N1-[2(R)-hydroxy-1(S)-indanyl]-N4-hydroxy-2(R)-[[4-(2,4-di-methoxy-phenyl)phenyl]methyl]butanediamide;
N1-[2(R)-hydroxy-1(S)-indanyl]-N4-hydroxy-2(R)-[[4-(3,5-di-chloro-phenyl)phenyl]methyl]butanediamide;
N1-[2(R)-hydroxy-1(S)-indanyl]-N4-hydroxy-2(R)-[[4-(2-trifluoromethyl-phenyl)phenyl]methyl]butanediamide;
N1-[2(R)-hydroxy-1(S)-indanyl]-N4-hydroxy-2(R)-[[4-(3-isopropyl-phenyl)phenyl]methyl]butanediamide;
N1-[2(R)-hydroxy-1(S)-indanyl]-N4-hydroxy-2(R)-[[4-(2,4-dichloro-phenyl)phenyl]methyl]butanediamide;
N1-[2(R)-hydroxy-1(S)-indanyl]-N4-hydroxy-2(R)-[[4-(3-chloro-4-fluoro-phenyl)phenyl]methyl]butanediamide;
N1-[2(R)-hydroxy-1(S)-indanyl]-N4-hydroxy-2(R)-[[4-(p-toluenesulfonyl-amino)phenyl]methyl]butanediamide;
N1-[2(R)-hydroxy-1(S)-indanyl]-N4-hydroxy-2(R)-phenylmethyl-3(S)-(tert-butyloxy-carbonyl-amino)-butanediamide;
N1-[2(R)-hydroxy-1(S)-indanyl]-N4-hydroxy-2(R)-[[4-(3,4-methylenedioxyphenyl)phenyl]methyl]-3(S)-(tert-butyloxy-carbonyl-amino)-butanediamide;
N1-[2(R)-hydroxy-1(S)-indanyl]-N4-hydroxy-2(R)-[[4-(3-methoxyphenyl)phenyl]methyl]butanediamide;
N1-[2(R)-hydroxy-1(S)-indanyl]-N4-hydroxy-2(R)-[[4-(3-fluorophenyl)phenyl]methyl]butanediamide;
N1-[2(R)-hydroxy-1(S)-indanyl]-N4-hydroxy-2(R)-[(3-fluoro-phenyl)methyl]-3(S)-(tert-butyloxy-carbonyl-amino)-butanediamide;
N1-[2(R)-hydroxy-1(S)-indanyl]-N4-hydroxy-2(R)-[(3-hydroxy-phenyl)methyl]-3(S)-(tert-butyloxy-carbonyl-amino)-butanediamide;
N1-[2(R)-hydroxy-1(S)-indanyl]-N4-hydroxy-2(R)-[[4-(3-nitrophenyl)phenyl]methyl]butanediamide;
N1-[2(R)-hydroxy-1(S)-indanyl]-N4-hydroxy-2(R)-[[4-(3-(methylsulfonyl-amino)-phenyl)phenyl]methyl]-butanediamide;
N1-[2(R)-hydroxy-1(S)-indanyl]-N4-hydroxy-2(R)-[(3-hydroxy-phenyl)methyl]-3(S)-(3-trimethylsilyl-propyl)-butanediamide;
N1-[2(R)-bydroxy-1(S)-indanyl]-N4-hydroxy-2(R)-[(3-hydroxy-phenyl)methyl]-3(S)-(2,2-dimethyl-propionamido)-butanediamide;
N1-[2(R)-hydroxy-1(S)-indanyl]-N4-hydroxy-2(R)-[(3-hydroxy-phenyl)methyl]-3(S)-(ethyloxy-carbonyl-amino)-butanediamide;
N1-[2(R)-hydroxy-1(S)-indanyl]-N4-hydroxy-2(R)-[(3-hydroxy-phenyl)methyl]-3 (S)-(iso-butyloxy-carbonyl-amino)-butanediamide;
N1-[2(R)-hydroxy-1(S)-indanyl]-N4-hydroxy-2(R)-[(3-hydroxy-phenyl)methyl]-3(S)-(propionamido)-butanediamide;
N1-[2(R)-hydroxy-1(S)-indanyl]-N4-hydroxy-2(R)-[(3-hydroxy-phenyl)methyl]-3(S)-(1-methyl-cyclopropane Carboxamido-1-yl)-butanediamide;
N1-[2(R)-hydroxy-1(S)-indanyl]-N4-hydroxy-2(R)-[(3-hydroxy-phenyl)methyl]-3(S)-(2,2-dimethylpropyl-amino)-butanediamide;
N1-[2(R)-hydroxy-1(S)-indanyl]-N4-hydroxy-2(R)-[(3-hydroxy-phenyl)methyl]-3(S)-(methylsulfonyl-amino)-butanediamide;
N1-[2(R)-hydroxy-1(S)-indanyl]-N4-hydroxy-2(R)-[(3-hydroxy-phenyl)methyl]-3(S)-amino-butanediamide;
N1-[2(R)-hydroxy-1(S)-indanyl]-N4-hydroxy-2(R)-[(4-(methylsulfonylamino)-phenyl)methyl]-butanediamide;
N1-[2(R)-hydroxy-1(S)-indanyl]-N4-hydroxy-2(R)-[(3-hydroxy-phenyl)methyl]-3(S)-(cyclobutane Carboxamido-1-yl)-butanediamide;
N1-[2(R)-hydroxy-1(S)-indanyl]-N4-hydroxy-2(R)-[(3-hydroxy-phenyl)methyl]-3(S)-(2-hydroxymethyl-isobutanamide)-butanediamide;
N1-[2(R)-hydroxy-1(S)-indanyl]-N4-hydroxy-2(R)-[(3-hydroxy-phenyl)methyl]-3(S)-(1-hydroxyl-cyclopropane Carboxamido-1-yl)-butanediamide;
N1-[2(R)-hydroxy-1(S)-indanyl]-N4-hydroxy-2(R)-[(3-hydroxy-phenyl)methyl]-3(S)-(1-phenyl-cyclopropane Carboxamido-1-yl)-butanediamide;
N1-[2(R)-hydroxy-1(S)-indanyl]-N4-hydroxy-2(R)-[(3-hydroxy-phenyl)methyl]-3(S)-(bezene Carboxamido-1-yl)-butanediamide;
N1-[2(R)-hydroxy-1(S)-indanyl]-N4-hydroxy-2(R)-[(3-hydroxy-phenyl)methyl]-3(S)-(1-cyano-cyclopropane Carboxamido-1-yl)-butanediamide;
N1-[2(R)-hydroxy-1(S)-indanyl]-N4-hydroxy-2(R)-[(3-hydroxy-phenyl)methyl]-3(S)-(1-phenyl-cyclopentane Carboxamido-1-yl)-butanediamide;
N1-[2(R)-hydroxy-1(S)-indanyl]-N4-hydroxy-2(R)-[(3-hydroxy-phenyl)methyl]-3(S)-(1-methyl-cyclohexane Carboxamido-1-yl)-butanediamide;
N1-[2(R)-hydroxy-1(S)-indanyl]-N4-hydroxy-2(R)-[(3-hydroxy-phenyl)methyl]-3(S)-(2-indole carboxamido)-butanediamide;
N1-[2(R)-hydroxy-1(S)-indanyl]-N4-hydroxy-2(R)-[(3-hydroxy-phenyl)methyl]-3(S)-(2-furan carboxamido)-butanediamide;
N1-[2(R)-hydroxy-1(S)-indanyl]-N4-hydroxy-2(R)-[(3-hydroxy-phenyl)methyl]-3(S)-(2-quinoline carboxamido)-butanediamide;
N1-[2(R)-hydroxy-1(S)-indanyl]-N4-hydroxy-2(R)-[(3-hydroxy-phenyl)methyl]-3(S)-(3,4,5-trimethoxy benzene Carboxamido-1-yl)-butanediamide;
N1-[2(R)-hydroxy-1(S)-indanyl]-N4-hydroxy-2(R)-[(3-hydroxy-phenyl)methyl]-3(S)-(2-methyl-3-amino-benzene Carboxamido-1-yl)-butanediamide;
N1-[2(R)-hydroxy-1(S)-indanyl]-N4-hydroxy-2(R)-[(3-hydroxy-phenyl)methyl]-3(S)-(2-methyl-6-amino-benzene Carboxamido-1-yl)-butanediamide;
N1-[2(R)-hydroxy-1(S)-indanyl]-N4-hydroxy-2(R)-[(3-hydroxy-phenyl)methyl]-3(S)-(3-pyridine Carboxamido-1-yl)-butanediamide;
N1-[2(R)-hydroxy-1(S)-indanyl]-N4-hydroxy-2(R)-[(3-hydroxy-phenyl)methyl]-3(S)-(1-(2,4-dichloro-phenyl)-cyclopropane Carboxamido-1-yl)-butanediamide;
N1-[2(R)-hydroxy-1(S)-indanyl]-N4-hydroxy-2(R)-[(3-hydroxy-phenyl)methyl]-3(S)-(1-(4-chloro-phenyl)-cyclopropane Carboxamido-1-yl)-butanediamide;
N1-[2(R)-hydroxy-1(S)-indanyl]-N4-hydroxy-2(R)-[(3-hydroxy-phenyl)methyl]-3(S)-(3-methylsulfonyl)-benzene Carboxamido-1-yl)-butanediamide;
N1-[2(R)-hydroxy-1(S)-indanyl]-N4-hydroxy-2(R)-[(3-hydroxy-phenyl)methyl]-3(S)-(2-methylsulfonyl-benzene Carboxamido-1-yl)-butanediamide;
N1-[2(R)-hydroxy-1(S)-indanyl]-N4-hydroxy-2(R)-[(3-hydroxy-phenyl)methyl]-3(S)-(3-cyano-benzene Carboxamido-1-yl)-butanediamide;
N1-[2(R)-hydroxy-1(S)-indanyl]-N4-hydroxy-2(R)-[(3-hydroxy-phenyl)methyl]-3(S)-(6-quinoline carboxamido)-butanediamide;
N1-[2(R)-hydroxy-1(S)-indanyl]-N4-hydroxy-2(R)-[(3-hydroxy-phenyl)methyl]-3(S)-(1-ethyl,3-methyl-pyrazole 5-carboxamido)-butanediamide;
N1-[2(R)-hydroxy-1(S)-indanyl]-N4-hydroxy-2(R)-[(3-hydroxy-phenyl)methyl]-3-(4-morpholino-benzene Carboxamido-1-yl)-butanediamide;
N1-[2(R)-hydroxy-1(S)-indanyl]-N4-hydroxy-2(R)-[(3-hydroxy-phenyl)methyl]-3(S)-(2-chloro-4-methylsulfonyl-benzene Carboxamido-1-yl)-butanediamide;
N1-[2(R)-hydroxy-1(S)-indanyl]-N4-hydroxy-2(R)-[(3-hydroxy-phenyl)methyl]-3(S)-(4-(imidazol-1-yl)benzene Carboxamido-1-yl)-butanediamide;
N1-[2(R)-hydroxy-1(S)-indanyl]-N4-hydroxy-2(R)-[(3-hydroxy-phenyl)methyl]-3(S)-(2-thiophene Carboxamido-1-yl)-butanediamide;
N1-[2(R)-hydroxy-1(S)-indanyl]-N4-hydroxy-2(R)-[(3-hydroxy-phenyl)methyl]-3(S)-(1-tert-butyl,3-methyl-pyrazole 5-carboxamido)-butanediamide;
N1-[2(R)-hydroxy-1(S)-indanyl]-N4-hydroxy-2(R)-[(3-hydroxy-phenyl)methyl]-3(S)-(4-aminomethyl benzene Carboxamido-1-yl)-butanediamide;
N1-[2(R)-hydroxy-1(S)-indanyl]-N4-hydroxy-2(R)-[(3-hydroxy-phenyl)methyl]-3(S)-(2-hydroxyl-isobutanamido)-butanediamide;
N1-[2(R)-hydroxy-1(S)-indanyl]-N4-hydroxy-2(R)-[(3-hydroxy-phenyl)methyl]-3(S)-(cyclopropane Carboxamido-1-yl)-butanediamide;
N1-[2(R)-hydroxy-1(S)-indanyl]-N4-hydroxy-2(R)-[(3-hydroxy-phenyl)methyl]-3 (S)-(cyclopentane Carboxamido-1-yl)-butanedi amide;
N1-[2(R)-hydroxy-1(S)-indanyl]-N4-hydroxy-2(R)-[(3-hydroxy-phenyl)methyl]-3(S)-(2-cyclopentyl acetamido)-butanediamide;
N1-[2(R)-hydroxy-1(S)-indanyl]-N4-hydroxy-2(R)-[(3-hydroxy-phenyl)methyl]-3(S)-(cyclohexane Carboxamido-1-yl)-butanediamide;
N1-[2(R)-hydroxy-1(S)-indanyl]-N4-hydroxy-2(R)-[(3-hydroxy-phenyl)methyl]-3(S)-(4-(4-N-Boc-piperazinyl-1-yl)benzene Carboxamido-1-yl)-butanediamide;
N1-[2(R)-hydroxy-1(S)-indanyl]-N4-hydroxy-2(R)-[(3-hydroxy-phenyl)methyl]-3(S)-(4-(piperazinyl-1-yl)benzene Carboxamido-1-yl)-butanediamide;
N1-[2(R)-hydroxy-1(S)-indanyl]-N4-hydroxy-2(R)-[(3-hydroxy-phenyl)methyl]-3(S)-(2-fluoro-6-chloro-benzene Carboxamido-1-yl)-butanediamide;
N1-[2(R)-hydroxy-1(S)-indanyl]-N4-hydroxy-2(R)-[(3-hydroxy-phenyl)methyl]-3 (S)-(1-amino-cyclohexane Carboxamido-1-yl)-butanediamide;
N1-[2(R)-hydroxy-1(S)-indanyl]-N4-hydroxy-2(R)-[(3-hydroxy-phenyl)methyl]-3(S)-(2-methylthio-acetamido)-butanediamide;
N1-[2(R)-hydroxy-1(S)-indanyl]-N4-hydroxy-2(R)-[(3-hydroxy-phenyl)methyl]-3(S)-(2-methoxy-acetamido)-butanediamide;
N1-[2(R)-hydroxy-1(S)-indanyl]-N4-hydroxy-2(R)-[(3-hydroxy-phenyl)methyl]-3(S)-(1-allyl-cyclopentane Carboxamido-1-yl)-butanediamide;
N1-[2(R)-hydroxy-1(S)-indanyl]-N4-hydroxy-2(R)-[(3-hydroxy-phenyl)methyl]-3(S)-(1-n-propyl-cyclopentane Carboxamido-1-yl)-butanediamide;
N1-[2(R)-hydroxy-1(S)-indanyl]-N4-hydroxy-2(R)-[(3-hydroxy-phenyl)methyl]-3(S)-(1-allyl-cyclopropane Carboxamido-1-yl)-butanediamide;
N1-[2(R)-hydroxy-1(S)-indanyl]-N4-hydroxy-2(R)-[(3-hydroxy-phenyl)methyl]-3(S)-(8-quinoline-sulfonamido)-butanediamide;
N1-[2(R)-hydroxy-1(S)-indanyl]-N4-hydroxy-2(R)-[(3-hydroxy-phenyl)methyl]-3(S)-(4-nitro-benzene sulfonamido)-butanediamide;
N1-[2(R)-hydroxy-1(S)-indanyl]-N4-hydroxy-2(R)-[(3-hydroxy-phenyl)methyl]-3(S)-(1,4-di-methyl-2-chloro-pyrazole-3-sulfonamido)-butanediamide;
N1-[2(R)-hydroxy-1(S)-indanyl]-N4-hydroxy-2(R)-[(3-hydroxy-phenyl)methyl]-3(S)-(1,5-dimethyl-isoxazole 3-sulfonamido)-butanediamide;
N1-[2(R)-hydroxy-1(S)-indanyl]-N4-hydroxy-2(R)-[(3-hydroxy-phenyl)methyl]-3(S)-(1-methyl-imidazole 3-sulfonamido)-butanediamide;
N1-[2(R)-hydroxy-1(S)-indanyl]-N4-hydroxy-2(R)-[(3-hydroxy-phenyl)methyl]-3(S)-(benzene sulfonamido)-butanediamide;
N1-[2(R)-hydroxy-1(S)-indanyl]-N4-hydroxy-2(R)-[(3-hydroxy-phenyl)methyl]-3(S)-(1,4-dimethyl pyrazole 3-sulfonamido)-butanediamide;
N1-[2(R)-hydroxy-1(S)-indanyl]-N4-hydroxy-2(R)-[(3-hydroxy-phenyl)methyl]-3(S)-(2-methylsulfonyl benzene sulfonamido-1-yl)-butanediamide;
N1-[2(R)-hydroxy-1(S)-indanyl]-N4-hydroxy-2(R)-[(3-hydroxy-phenyl)methyl]-3(S)-(cyclohexylamino)-butanediamide;
N1-[2(R)-hydroxy-1(S)-indanyl]-N4-hydroxy-2(R)-[(3-hydroxy-phenyl)methyl]-3(S)-(iso-propylamino)-butanediamide;
N1-[2(R)-hydroxy-1(S)-indanyl]-N4-hydroxy-2(R)-[4-(2-trifluoromethylphenyl)-phenylmethyl]-3(S)-(2,2-dimethylpropyl-amino)-butanediamide;
N1-[2(R)-hydroxy-1(S)-indanyl]-N4-hydroxy-2(R)-[(3-hydroxy-phenyl)methyl]-3(S)-(cyclopentylamino)-butanediamide;
N1-[2(R)-hydroxy-1(S)-indanyl]-N4-hydroxy-2(R)-[(3-hydroxy-phenyl)methyl]-3(S)-(cyclopropylmethyl)-butanediamide;
N1-[2(R)-hydroxy-1(S)-indanyl]-N4-hydroxy-2(R)-[(3-hydroxy-phenyl)methyl]-3(S)-(benzylamino)-butanediamide; N1-[2(R)-hydroxy-1(S)-indanyl]-N4-hydroxy-2(R)-[(3-hydroxy-phenyl)methyl]-3(S)-(2-furanylmethylamino)-butanediamide;
N1-[2(R)-hydroxy-1(S)-indanyl]-N4-hydroxy-2(R)-[(3-hydroxy-4-methylphenyl)methyl]-3(S)-(3-cyanophenylmethylamino)-butanediamide;
N1-[2(R)-hydroxy-1(S)-indanyl]-N4-hydroxy-2(R)-[(3-hydroxy-phenyl)methyl]-3(S)-(2,2-dimethylpropyl-amino)-butanediamide;
N1-[2(R)-hydroxy-1(S)-indanyl]-N4-hydroxy-2(R)-[(3-hydroxy-phenyl)methyl]-3(S)-(2-pentylamino)-butanediamide;
N1-[2(R)-hydroxy-1(S)-indanyl]-N4-hydroxy-2(R)-[(3-hydroxy-phenyl)methyl]-3(S)-(bis-cyclopropylmethylamino)-butanediamide;
N1-[2(R)-hydroxy-1(S)-indanyl]-N4-hydroxy-2(R)-[(3-hydroxy-phenyl)methyl]-3(S)-(2-thiophenylmethylamino)-butanediamide;
N1-[2(R)-hydroxy-1(S)-indanyl]-N4-hydroxy-2(R)-[(3-hydroxy-phenyl)methyl]-3( )-(2-methyl-propylamino)-butanediamide;

or a pharmaceutically acceptable salt form or a steroisomer thereof.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g. monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Embodiments of pharmaceutical compositions of the present invention comprise a replication defective recombinant viral vector encoding the polynucleotide inhibitory agent of the present invention and a transfection enhancer, such as poloxamer. An example of a poloxamer is Poloxamer 407, which is commercially available (BASF, Parsippany, N.J.) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The active expression-inhibiting agents may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The present invention also provides methods of inhibiting the processing of amyloid-beta precursor protein in a subject suffering or susceptible to the abnormal processing of said protein, which comprise the administration to said subject a therapeutically effective amount of an expression-inhibiting agent of the invention. Another aspect of the present method invention is the treatment or prevention of a condition involving cognitive impairment or a susceptibility to the condition. A special embodiment of this invention is a method wherein the condition is Alzheimer's disease.

As defined above, therapeutically effective dose means that amount of protein, polynucleotide, peptide, or its antibodies, agonists or antagonists, which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The pharmaceutical compositions according to this invention may be administered to a subject by a variety of methods. They may be added directly to target tissues, complexed with cationic lipids, packaged within liposomes, or delivered to target cells by other methods known in the art. Localized administration to the desired tissues may be done by catheter, infusion pump or stent. The DNA, DNA/vehicle complexes, or the recombinant virus particles are locally administered to the site of treatment. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Examples of ribozyme delivery and administration are provided in Sullivan et al. WO 94/02595.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides will be specific to particular cells, conditions, locations, etc.

As discussed hereinabove, recombinant viruses may be used to introduce DNA encoding polynucleotide agents useful in the present invention. Recombinant viruses according to the invention are generally formulated and administered in the form of doses of between about 10⁴ and about 10¹⁴ pfu. In the case of AAVs and adenoviruses, doses of from about 10⁶ to about 10¹¹ pfu are preferably used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

Still another aspect or the invention relates to a method for diagnosing a pathological condition involving cognitive impairment or a susceptibility to the condition in a subject, comprising determining the amount of polypeptide comprising an amino acid sequence selected from the group consisting of SEQ ID NO: 7, 8, 9, and 10 in a biological sample, and comparing the amount with the amount of the polypeptide in a healthy subject, wherein an increase of the amount of polypeptide compared to the healthy subject is indicative of the presence of the pathological condition.

### EXPERIMENTAL SECTION

### Example 1

### Screening for Proteases that Modulate Amyloid Beta 1-42 Levels

To identify novel drug targets that change the APP processing, stable cell lines over expressing APP are made by transfecting Hek293 or SH-SY5Y cells with APP770 wt cDNA cloned into pcDNA3.1, followed by selection with G418 for 3 weeks. At this time point colonies are picked and stable clones are expanded and tested for their secreted amyloid-beta peptide levels. The cell lines designated as “Hek293 APPwt” and “SH-SY5Y APPwt” are used in the assays.

Hek293 APPwt Assay: Cells seeded in collagen-coated plates at a cell density of 15000 cells/well (384 well plate) in DMEM (10% FBS), are infected 24 h later with 1 μl or 0.2 μl of adenovirus (corresponding to an average multiplicity of infection (MOI) of 120 and 24 respectively). The following day, the virus is washed away and DMEM (25 mM Hepes; 10% FBS) is added to the cells. Amyloid-beta peptides are allowed to accumulate during 24 h.

SH-SY5Y APPwt Assay: Cells are seeded in collagen-coated plates at a cell density of 15000 cells/well (384 well plate) in Dulbecco's MEM with Glutamax I+15% FBS HI+non-essential amino acids+Geneticin 500 μg/ml. The cells are differentiated towards the neuronal phenotype by adding 9-cis retinoic acid to a final concentration of 1 μM on day 1, day 3, day 5 and day 8. On day 9, the cells are infected with 1 μl of adenovirus (corresponding to an average multiplicity of infection (MOI) of 120 respectively). The following day, the virus is washed away and DMEM 25 mM Hepes 10% FBS is added to the cells. Amyloid beta peptides are allowed to accumulate for 24 h.

ELISA: The ELISA plate is prepared by coating with a capture antibody (JRF/cAbeta42/26) (the antibody recognizes a specific epitope on the C-terminus of Abeta 1-42; obtained from M Mercken, Johnson and Johnson Pharmaceutical Research and Development, B-2340 Beerse, Belgium) overnight in buffer 42 (Table 2) at a concentration of 2.5 μg/ml. The excess capture antibody is washed away the next morning with PBS and the ELISA plate is then blocked overnight with casein buffer (see Table 2) at 4° C. Upon removal of the blocking buffer, 30 μl of the sample is transferred to the ELISA plate and incubated overnight at 4° C. After extensive washing with PBS-Tween20 and PBS, 30 μl of the horseradish peroxidase (HRP) labeled detection antibody (Peroxidase Labeling Kit, Roche), JRF/AbetaN/25-HRP (obtained from M Mercken, Johnson and Johnson Pharmaceutical Research and Development, B-2340 Beerse, Belgium) is diluted 1/5000 in buffer C (see Table 2) and added to the wells for another 2 h. Following the removal of excess detection antibody by a wash with PBS-Tween20 and PBS, HRP activity is detected via addition of luminol substrate (Roche), which is converted into a chemiluminescent signal by the HRP enzyme.

In addition, for the SH-SY5Y APPwt assay, the samples are also analyzed in an amyloid beta x-42 ELISA. This ELISA detects all amyloid beta peptide species ending at position 42, comprising 1-42, 11-42 and 17-42 (p3), which originate respectively from BACE activity at position 1 and 11, and alpha secretase activity at position 17. Thus, in addition to the amyloidogenic pathway, the non-amyloidogenic pathway is also monitored. The protocol for the Abeta x-42 ELISA is identical to the protocol for the Abeta 1-42 ELISA, except that a HRP labeled 4G8 antibody (Signet; the antibody recognizes a specific epitope in the center of the Abeta peptides) is used as detection antibody.

TABLE 2
Buffers And Solutions Used For ELISA

| | |
|---|---|
| Buffer 42 | 30 mM NaHCO₃, 70 mM Na₂CO₃, 0.05% NaN₃, pH9.6 |
| Casein | 0.1% casein in PBS 1x |
| buffer | |
| EC Buffer | 20 mM sodium phosphate, 2 mM EDTA, 400 mM NaCl, |
| | 0.2% BSA, 0.05% CHAPS, 0.4% casein, 0.05% NaN₃, pH7 |
| Buffer C | 20 mM sodium phosphate, 2 mM EDTA, 400 mM NaCl, 1% |
| | BSA, pH7 |
| PBS 10x | 80 g NaCl + 2 g KCl + 11.5 g Na₂HPO₄.7H₂O + 2 g KH₂PO₄ |
| | in 11 milli Q, pH 7.4 |
| PBST | PBS 1x with 0.05% Tween 20 |

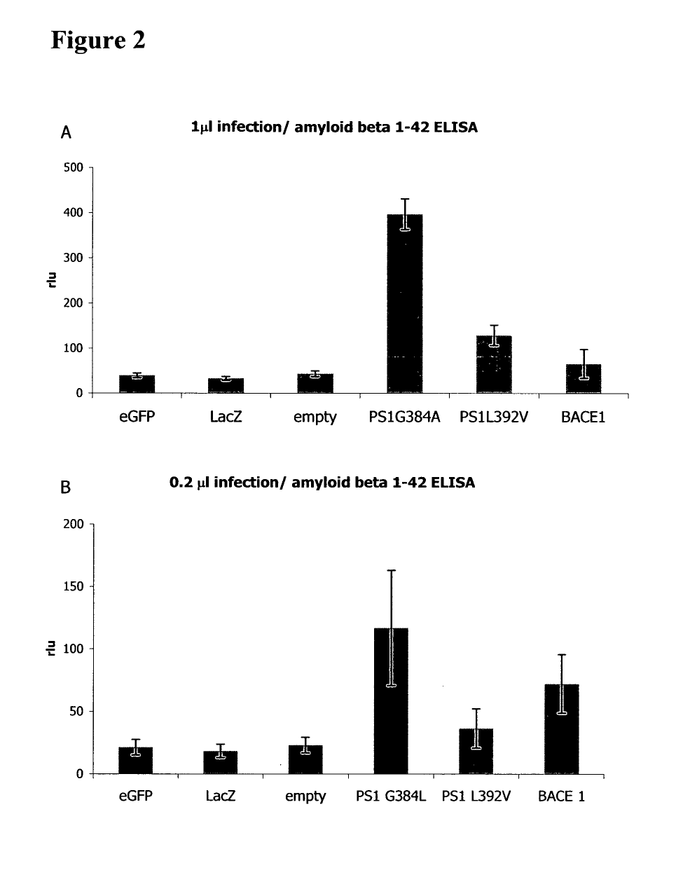

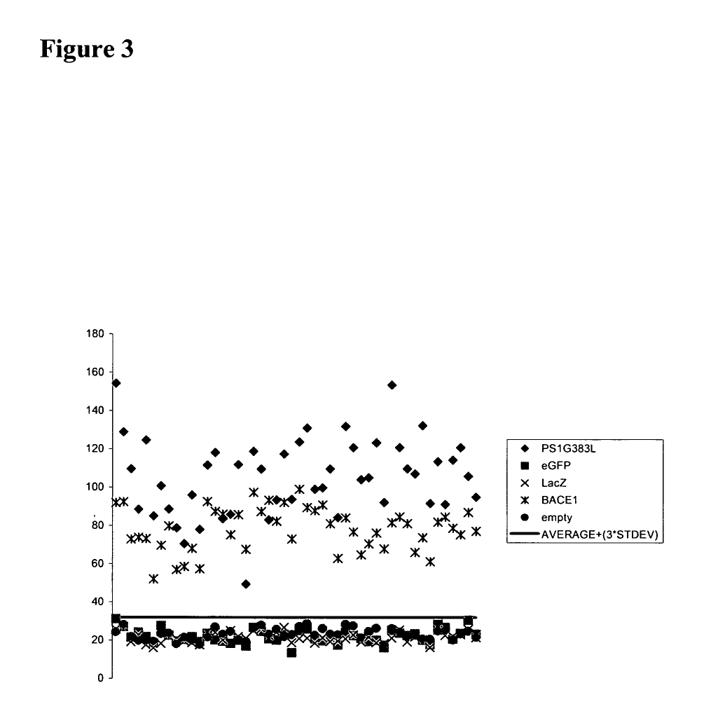

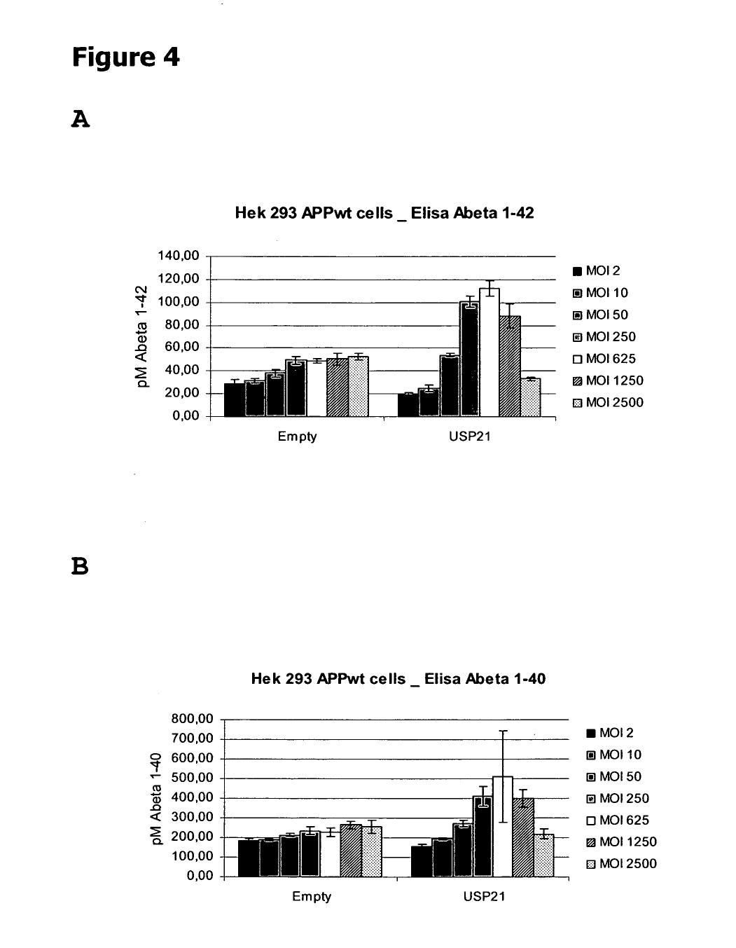

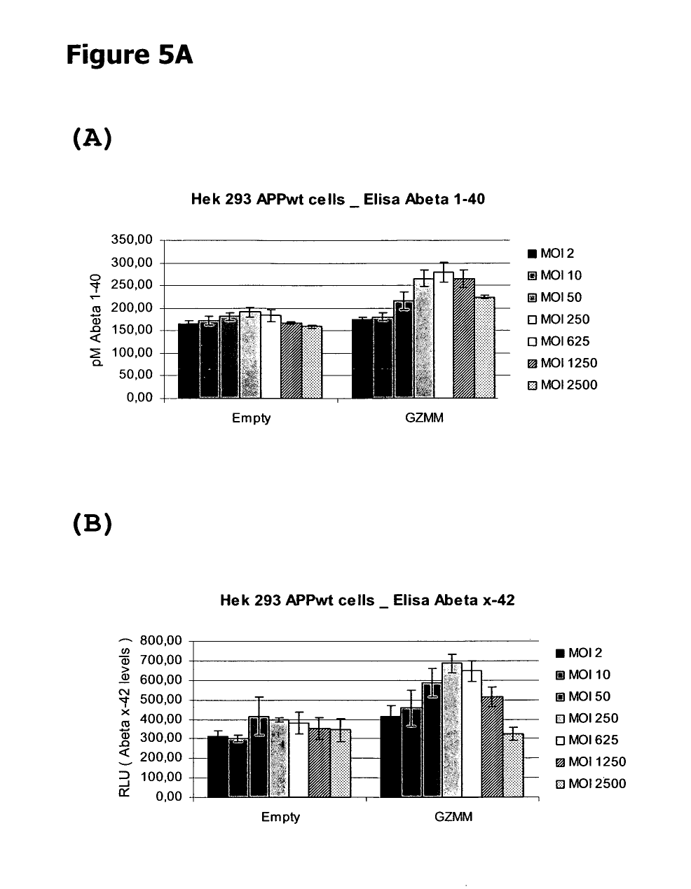

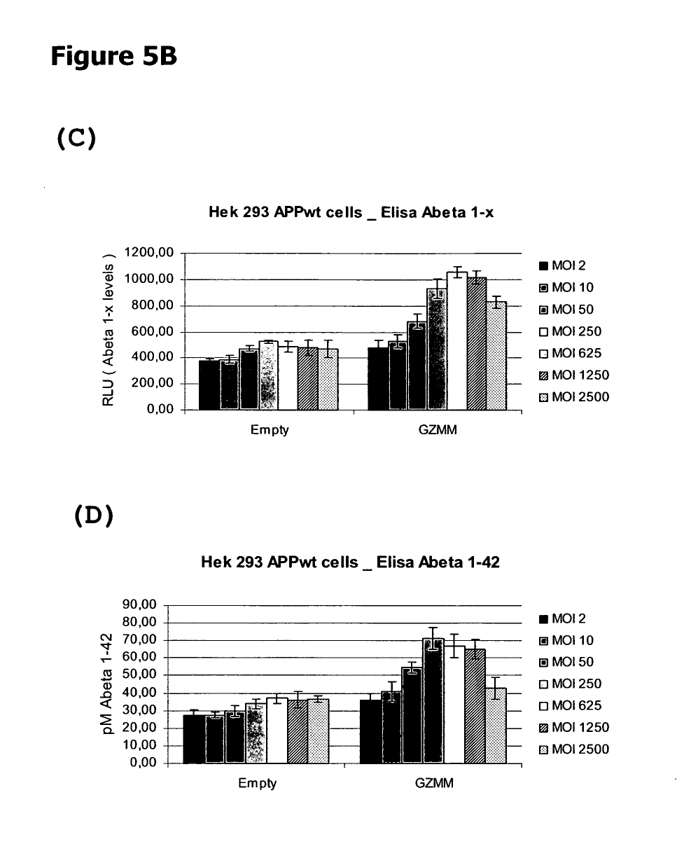

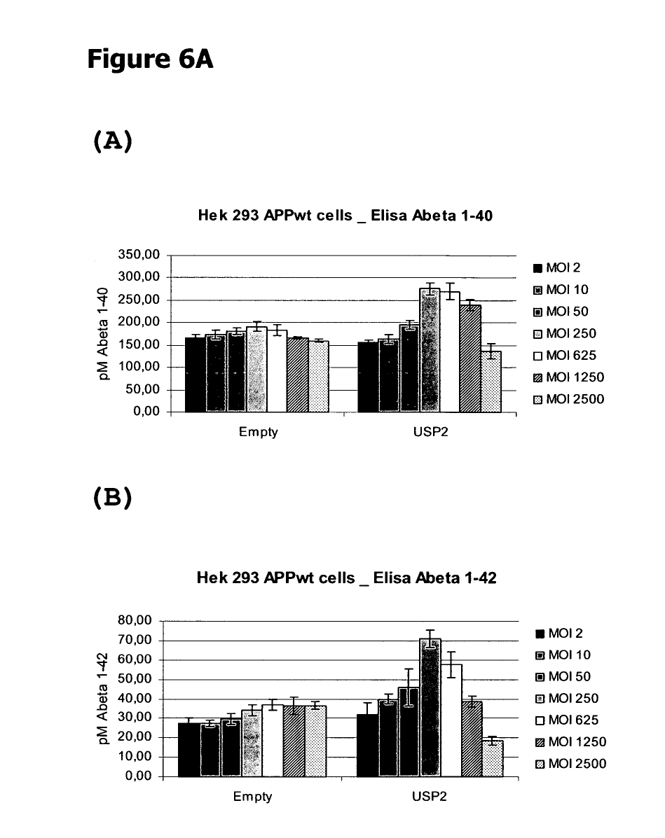

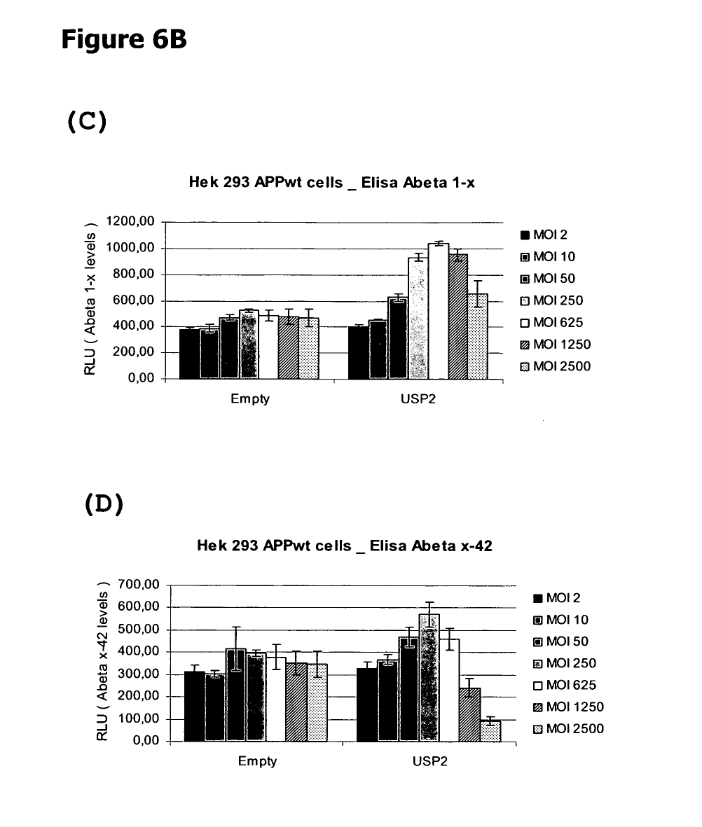

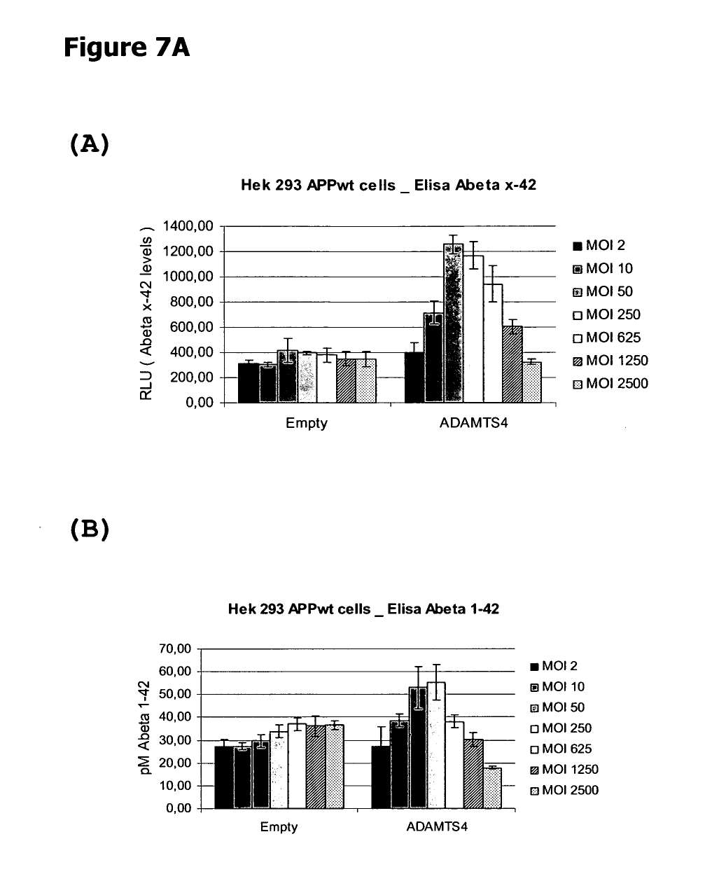

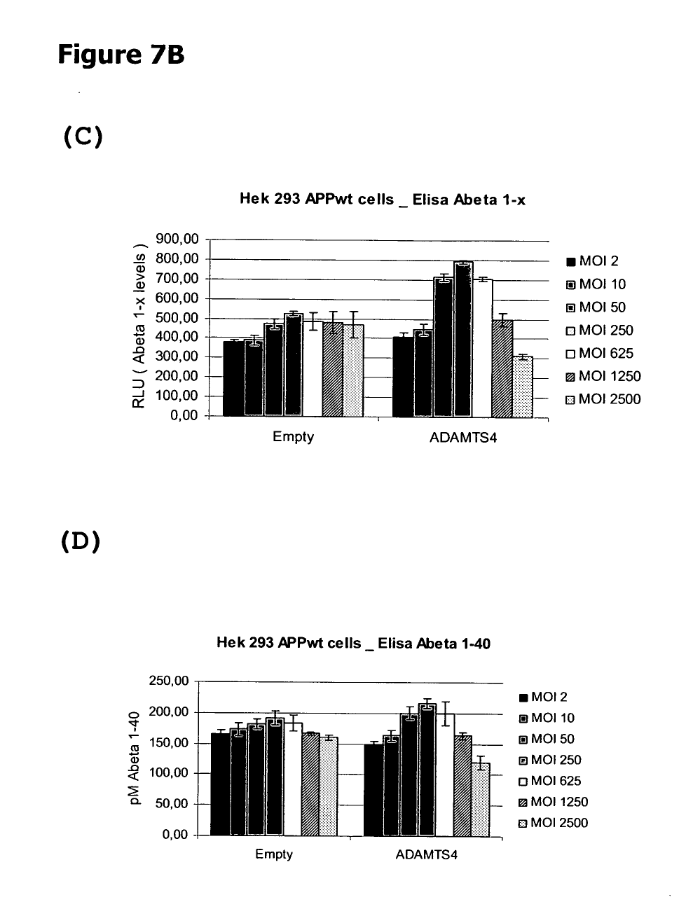

To validate the assay, the effect of adenoviral over expression with random titer of two clinical PS1 mutants and BACE on amyloid beta 1-42 production is evaluated in the Hek293 APPwt cells. As is shown in FIG. 2, all PS1 and BACE constructs induce amyloid beta 1-42 levels as expected. As is shown in FIG. 3, adenoviral overexpression of the clinical PS1 mutants in the SH-SY5Y APPwt cells also yield a significant induction of amyloid beta 1-42 levels. However, since overexpression of BACE in the SH-SY5Y APPwt cells do not result in an induction of amyloid beta 1-42 levels, amyloid beta x-42 levels are determined and show a clear induction.

An adenoviral cDNA library is constructed as follows. DNA fragments covering the full coding region of the target candidate genes are amplified by PCR from a pooled placental and fetal liver cDNA library (InvitroGen). All fragments are cloned into an adenoviral vector as described in U.S. Pat. No. 6,340,595, the contents of which are herein incorporated by reference, and subsequently adenoviruses are made harboring the corresponding cDNAs. The screen types using these libraries are presented in Table 3.

TABLE 3

| Screen number | Cell type | ELISA | Adenoviral library |
|---|---|---|---|
| H25 | Hek293 APPwt | Abeta 1-42 | KI-library |
| H22 | SH-SY5Y APPwt | Abeta 1-42 | KI-library |
| H28 | SH-SY5Y APPwt | Abeta x-42 | KI-library |

Hek293 APPwt and SH-SY5Y APPwt cells are infected with indicated volumes of the adenoviral cDNA library and Abeta 1-42 or Abeta x-42 levels are determined. Activators of amyloid beta production are selected by calculating the average and standard deviation of all data points during the screening run (i.e. all plates processed in one week) and applying the formula AVERAGE+(N×STDEV) to calculate the cut off value (N is determined individually for every screen and is indicated in Tables 4A-4D). The average and standard deviation of all data points of the screening run was calculated and positives were selected as those cDNAs that score lower than AVERAGE−(N×STDEV) or higher than AVERAGE+(N×STDEV). The N values that are used to select the positives, differ from screening to screening, because of the different characteristics of the assays. These N values are indicated in the Table 4 (Act is activator, Rep is repressor). Whether a gene is a hit or no hit is indicated in the table respectively as the number 1 or 0. The data are represented as times (AVERAGE+(1×STDEV)). PS and RS represent respectively primary screen and rescreen, which is a duplicate of the primary screen. Therefore 4 data points are obtained for every type of screen. A cDNA is considered a hit when at least 2 data points score positive out of 4.

During the screening of the adenoviral library in the HEK293 APPwt cells, over expression of a number of protease cDNAs lead to increased levels of amyloid beta 1-42 peptides in the conditioned medium of HEK293 APPwt cells.

TABLE 4 A

| screen | H25 | | | | | | | |
|---|---|---|---|---|---|---|---|---|
| infection | 0.25 μl | | | | 1 μl | | | |
| N for Act | 3 | | | | 3 | | | |
| N for Rep | −1.6 | | | | −1.6 | | | |
| cDNA | PS | | RS | | PS | | RS | |
| APP | 4.417 | 5.43 | 4.813 | 3.219 | 5.479 | 3.515 | 1.473 | 3.729 |
| | 1 | 1 | 1 | 1 | 1 | 1 | 0 | 1 |
| GZMM | 2.783 | 3.979 | 3.378 | 2.252 | 2.951 | 4.46 | 0.312 | 2.75 |
| | 0 | 1 | 1 | 0 | 0 | 1 | 0 | 0 |
| USP2 | 0.544 | −0.013 | 1.832 | 1.795 | 2.971 | 3.869 | 1.473 | 3.034 |
| | 0 | 0 | 0 | 0 | 0 | 1 | 0 | 1 |
| ENSG00000117094 | 2.875 | 2.898 | 4.554 | 4.65 | 3.286 | 3.433 | 4.146 | 4.091 |
| | 0 | 0 | 1 | 1 | 1 | 1 | 1 | 1 |
| ADAMTS4 | 0.128 | 0.522 | 0.696 | 0.243 | −0.419 | −0.543 | −0.486 | −0.672 |
| | 0 | 0 | 0 | 0 | 0 | 0 | 0 | 0 |
| USP21 | 5.118 | 6.018 | 4.468 | 1.449 | 1.481 | 6.015 | 6.658 | 3.401 |
| | 1 | 1 | 1 | 0 | 0 | 1 | 1 | 0 |

TABLE 4B

| screen | H22 | H28 |
|---|---|---|
| infection | 1 μl | 1 μl |
| N for Act | 3 | 3 |
| N for Rep | −1.6 | −1.6 |

| cDNA | PS | | RS | | PS | | RS | |
|---|---|---|---|---|---|---|---|---|
| APP | 6.896 | 5.065 | 9.373 | 7.186 | 10.913 | 9.454 | 16.049 | 15.715 |
| | 1 | 1 | 1 | 1 | 1 | 1 | 1 | 1 |
| GZMM | −0.326 | −0.517 | 0.132 | −0.759 | −0.587 | −0.25 | 0.009 | −0.928 |
| | 0 | 0 | 0 | 0 | 0 | 0 | 0 | 0 |
| USP2 | 5.18 | 3.153 | 3.123 | 2.396 | 0.914 | 1.541 | 4.618 | 3.803 |
| | 1 | 1 | 1 | 0 | 0 | 0 | 1 | 1 |
| ENSG00000117094 | 1.291 | −0.077 | 0.272 | −0.793 | 0.198 | −0.181 | 1.578 | 0.766 |
| | 0 | 0 | 0 | 0 | 0 | 0 | 0 | 0 |
| ADAMTS4 | −1.401 | −2.05 | −0.466 | −1.344 | 1.252 | 1.389 | 3.8 | 3.368 |
| | 0 | 1 | 0 | 0 | 0 | 0 | 1 | 1 |
| USP21 | −0.119 | 1.727 | | | 0.58 | 0.517 | | |
| | 0 | 0 | | | 0 | 0 | | |

TABLE 4C

| Screening | H22 | | | | H25 | | | |
|---|---|---|---|---|---|---|---|---|
| Infection | 1 μl | | | | 1 μl | | | |
| N for Act | 3 | | | | 3 | | | |
| | DS | | PS | | DS | | PS | |
| cDNA | A | B | A | B | A | B | A | B |
| CDKN1A | 0.745 | 0.688 | 0.942 | 1.251 | 4.109 | 3.204 | 3.664 | 2.693 |
| | 0 | 0 | 0 | 0 | 1 | 1 | 1 | 0 |
| CSNK1G1 | 0.321 | 1.572 | −0.826 | −0.283 | 3.382 | 2.535 | 4.455 | 3.594 |
| | 0 | 0 | 0 | 0 | 1 | 0 | 1 | 1 |
| DGKE | 1.639 | 1.859 | −1.241 | −0.449 | 3.112 | 2.406 | 3.478 | 1.707 |
| | 0 | 0 | 0 | 0 | 1 | 0 | 1 | 0 |
| hRAS | 6.612 | 2.409 | 7.157 | 8.608 | 3.926 | 2.727 | 2.842 | 3.504 |
| | 1 | 0 | 1 | 1 | 1 | 0 | 0 | 1 |
| NR4A1 | −0.003 | 0.75 | −0.691 | 0.101 | 1.011 | 1.423 | 0.152 | 0.756 |
| | 0 | 0 | 0 | 0 | 0 | 0 | 0 | 0 |
| PREP | 0.779 | 1.562 | −0.517 | −0.433 | 3.554 | 2.623 | 4.121 | 4.455 |
| | 0 | 0 | 0 | 0 | 1 | 0 | 1 | 1 |
| PTPN6 | 1.701 | 1.2 | 1.778 | 1.854 | 4.409 | 3.371 | 4.052 | 2.828 |
| | 0 | 0 | 0 | 0 | 1 | 1 | 1 | 0 |
| SPINT1 | 4.007 | 1.396 | 2.169 | 2.344 | 4.196 | 3.282 | 1.866 | 2.209 |
| | 1 | 0 | 0 | 0 | 1 | 1 | 0 | 0 |
| SPC18 | 0.529 | −0.541 | 0.692 | 0.895 | 5.89 | 4.161 | 5.177 | 5.073 |
| | 0 | 0 | 0 | 0 | 1 | 1 | 1 | 1 |
| IMMP2L | −0.419 | 0.817 | 1.338 | 0.925 | 2.97 | 2.635 | 1.551 | 0.085 |
| | 0 | 0 | 0 | 0 | 0 | 0 | 0 | 0 |
| LOC166867 | 0.997 | 0.703 | 0.999 | 0.376 | 2.471 | 2.752 | 3.105 | 1.75 |
| | 0 | 0 | 0 | 0 | 0 | 0 | 1 | 0 |
| LOC148293 | 1.433 | 0.906 | 1.483 | 1.392 | 4.137 | 3.933 | 3.731 | 2.88 |
| | 0 | 0 | 0 | 0 | 1 | 1 | 1 | 0 |
| PSMA2 | 0.078 | −0.556 | 1.211 | 2.086 | 2.188 | 2.279 | 2.338 | 2.195 |
| | 0 | 0 | 0 | 0 | 0 | 0 | 0 | 0 |
| C14orf132 | 1.295 | 0.968 | −0.625 | −0.234 | 3.295 | 2.334 | 4.237 | 2.287 |
| | 0 | 0 | 0 | 0 | 1 | 0 | 1 | 0 |
| MAP3K8 | 0.893 | 3.729 | 0.228 | −0.006 | 0.949 | 0.851 | 0.147 | −0.55 |
| | 0 | 1 | 0 | 0 | 0 | 0 | 0 | 0 |
| NDUFA10 | 0.651 | 1.2 | 1.067 | 0.113 | 4.131 | 3.186 | 4.116 | 2.717 |
| | 0 | 0 | 0 | 0 | 1 | 1 | 1 | 0 |
| DAPK2 | 0.976 | 2.112 | −0.437 | −0.277 | 2.167 | 1.278 | 4.054 | 3.181 |
| | 0 | 0 | 0 | 0 | 0 | 0 | 1 | 1 |
| MAPK10 | 0.762 | 1.899 | −1.2 | −0.572 | 3.325 | 2.427 | 4.345 | 3.281 |
| | 0 | 0 | 0 | 0 | 1 | 0 | 1 | 1 |
| PDGFC | 4.195 | 1.399 | 3.549 | 2.683 | −0.524 | −0.406 | −0.381 | −0.143 |
| | 1 | 0 | 1 | 0 | 0 | 0 | 0 | 0 |
| NR1D2 | −1.9 | −0.707 | −0.779 | −0.612 | 1.064 | 3.277 | 1.599 | 2.383 |
| | 0 | 0 | 0 | 0 | 0 | 1 | 0 | 0 |
| Screening | H25 | | | | H28 | | | |
| Infection | 0.20 μl | | | | 1 μl | | | |
| N for Act | 3 | | | | 3 | | | |
| | DS | | PS | | DS | | PS | |
| cDNA | A | B | A | B | A | B | A | B |
| CDKN1A | 2.994 | 3.59 | 1.267 | 0.511 | −0.568 | 0.007 | −0.009 | 1.455 |
| | 0 | 1 | 0 | 0 | 0 | 0 | 0 | 0 |
| CSNK1G1 | 1.866 | 2.476 | 1.102 | 2.401 | 1.794 | 2.483 | 2.294 | 2.232 |
| | 0 | 0 | 0 | 0 | 0 | 0 | 0 | 0 |
| DGKE | 1.563 | 1.682 | 1.178 | 2.33 | 2.695 | 2.063 | 0.778 | 1.483 |
| | 0 | 0 | 0 | 0 | 0 | 0 | 0 | 0 |
| hRAS | 5.632 | 5.814 | 4.743 | 2.714 | 7.952 | 4.047 | 8.338 | 8.311 |
| | 1 | 1 | 1 | 0 | 1 | 1 | 1 | 1 |
| NR4A1 | 3.278 | 3.747 | 1.959 | 3.16 | 0.328 | 0.04 | −0.615 | 0.212 |
| | 1 | 1 | 0 | 1 | 0 | 0 | 0 | 0 |
| PREP | 1.87 | 3.003 | 1.79 | 3.252 | 1.918 | 2.949 | 0.79 | 0.838 |
| | 0 | 1 | 0 | 1 | 0 | 0 | 0 | 0 |
| PTPN6 | 3.91 | 5.114 | 2.395 | 2.218 | −0.563 | 0.563 | 0.487 | 1.19 |
| | 1 | 1 | 0 | 0 | 0 | 0 | 0 | 0 |
| SPINT1 | 2.546 | 2.364 | 1.671 | 0.848 | 1.509 | 0.355 | 2.072 | 0.572 |
| | 0 | 0 | 0 | 0 | 0 | 0 | 0 | 0 |
| SPC18 | 5.84 | 6.34 | 3.589 | 3.743 | −1.75 | −1.288 | −1.033 | −0.371 |
| | 1 | 1 | 1 | 1 | 0 | 0 | 0 | 0 |
| IMMP2L | 3.267 | 3.827 | 1.604 | 4.08 | −1.128 | −0.77 | −0.165 | 0.185 |
| | 1 | 1 | 0 | 1 | 0 | 0 | 0 | 0 |
| LOC166867 | 3.798 | 4.32 | 1.493 | 1.359 | −0.916 | 0.09 | 0.086 | −0.433 |
| | 1 | 1 | 0 | 0 | 0 | 0 | 0 | 0 |
| LOC148293 | 4.191 | 5.176 | 2.125 | 1.941 | −0.236 | 0.394 | 0.603 | 1.027 |
| | 1 | 1 | 0 | 0 | 0 | 0 | 0 | 0 |
| PSMA2 | 3.593 | 3.935 | 1.642 | 1.633 | −1.335 | −1.115 | 0.2 | 0.782 |
| | 1 | 1 | 0 | 0 | 0 | 0 | 0 | |
| C14orf132 | 1.65 | 1.771 | 2.037 | 2.897 | 2.804 | 1.991 | 0.789 | 2.421 |
| | 0 | 0 | 0 | 0 | 0 | 0 | 0 | 0 |
| MAP3K8 | 0.468 | 0.153 | 1.258 | 1.049 | 2.39 | 4.373 | 4.67 | 3.948 |
| | 0 | 0 | 0 | 0 | 0 | 1 | 1 | 1 |
| NDUFA10 | 1.126 | 2.041 | 2.54 | 3.035 | 0.939 | 1.204 | 0.835 | 0.031 |
| | 0 | 0 | 0 | 1 | 0 | 0 | 0 | 0 |
| DAPK2 | 0.972 | 1.959 | 0.765 | 1.7 | 2.208 | 4.197 | 2.578 | 3.677 |
| | 0 | 0 | 0 | 0 | 0 | 1 | 0 | 1 |
| MAPK10 | 1.606 | 2.086 | 1.281 | 2.667 | 2.271 | 2.804 | 0.897 | 1.36 |
| | 0 | 0 | 0 | 0 | 0 | 0 | 0 | 0 |
| PDGFC | −0.254 | −0.216 | −0.13 | −0.768 | 2.68 | 1.696 | 2.405 | 0.321 |
| | 0 | 0 | 0 | 0 | 0 | 0 | 0 | 0 |
| NR1D2 | 0.475 | 1.826 | 3.81 | 4.256 | −1.461 | −0.521 | −1.408 | −1.875 |
| | 0 | 0 | 1 | 1 | 0 | 0 | 0 | 0 |

TABLE 4D

| Screening | H22 | | | | H25 | | | |
|---|---|---|---|---|---|---|---|---|
| Infection | 1 μl | | | | 1 μl | | | |
| N for Rep | 2 | | | | 1.7 | | | |
| | DS | | PS | | DS | | PS | |
| cDNA | A | B | A | B | A | B | A | B |
| HTR2B | 1.834 | 1.621 | 2.767 | 1.436 | −1.961 | −1.72 | −1.407 | −1.273 |
| | 0 | 0 | 0 | 0 | 1 | 1 | 0 | 0 |
| MARK1 | −1.479 | 0.173 | −0.429 | −0.688 | −1.76 | −1.794 | −1.674 | −1.641 |
| | 0 | 0 | 0 | 0 | 1 | 1 | 0 | 0 |
| PIP5K1A | −1.517 | −0.59 | −1.113 | −0.974 | −1.473 | −1.104 | −1.721 | −1.978 |
| | 0 | 0 | 0 | 0 | 0 | 0 | 1 | 1 |
| Screening | H22 | | | | H25 | | | |
| Infection | 1 μl | | | | 1 μl | | | |
| N for Rep | 2 | | | | 2 | | | |
| | DS | | PS | | DS | | PS | |
| cDNA | A | B | A | B | A | B | A | B |
| FLJ23516 | −2.339 | −2.611 | −2.091 | −2.397 | −2.348 | −2.449 | −2.114 | −2.15 |
| | 0 | 1 | 1 | 1 | 1 | 1 | 1 | 1 |
| Screening | H25 | | | | H28 | | | |
| Infection | 0.25 μl | | | | 1 μl | | | |
| N for Rep | 2 | | | | 3 | | | |
| | DS | | PS | | DS | | PS | |
| cDNA | A | B | A | B | A | B | A | B |
| HTR2B | −0.838 | −0.848 | −0.733 | −0.8 | 3.959 | 4.254 | 1.821 | 1.523 |
| | 0 | 0 | 0 | 0 | 1 | 1 | 0 | 0 |
| MARK1 | −1.891 | −2.024 | −1.684 | −1.51 | −1.205 | 0.496 | 0.911 | 0.314 |
| | 0 | 0 | 0 | 0 | 0 | 0 | 0 | 0 |
| PIP5K1A | −0.504 | −1.216 | −0.996 | −1.114 | −1.426 | −1.209 | −1.33 | −1.733 |
| | 0 | 0 | 0 | 0 | 0 | 0 | 0 | 0 |
| Screening | H25 | | | | H28 | | | |
| Infection | 0.25 μl | | | | 1 μl | | | |
| N for Rep | 1.5 | | | | 2.5 | | | |
| | DS | | PS | | DS | | PS | |
| cDNA | A | B | A | B | A | B | A | B |
| FLJ23516 | −2.421 | −2.545 | −1.803 | −1.697 | −2.571 | −3.09 | −1.51 | −1.376 |
| | 1 | 1 | 1 | 1 | 1 | 1 | 0 | 0 |

All cDNAs scoring higher then the cut off value are considered as positives and thus modulate amyloid beta 1-42 levels. This is validated infecting Hek293APPwt cells with a control plate containing PS1G384A, BACE1 and eGFP, empty and LacZ adenoviruses. The average and standard deviation are calculated based upon the negative controls. Applying the cut off (AVERAGE+(3×STDEV)) reveals that all positive controls are identified as hits (FIG. 3). Repressors of the amyloid beta production are selected in a similar way, except that the cDNAs have to score lower than the cut off value determined by the formula AVERAGE−(N×STDEV). The same procedure applies for the SH-SY5Y APPwt cells. One of the selected activators during the screen is APP, underscoring the relevance of the identified hits.

The proteases and proteases identified in the aforesaid screen as involved in the up-regulation of amyloid beta 1-42 are listed in Table 5 below.

TABLE 5

| | | | SEQ ID NO: | | |
|---|---|---|---|---|---|
| Accession | Description | Code | DNA | Protein | KD |
| NM_012475 | ubiquitin specific protease 21 | USP21 | 1 | 7 | 14-21; |
| | | | | | 427-470 |
| NM_005317 | granzyme M | GZMM | 2 | 8 | 26-28; |
| | | | | | 389-396 |
| NM_004205 | ubiquitin specific protease 2 | USP2 | 3 | 9 | 22-25; |
| | | | | | 397-426 |
| NM_005099 | a disintegrin-like and | ADAMTS4 | 4 | 10 | 29-32; |
| | metalloprotease (reprolysin | | | | 332-388 |
| | type) with thrombospondin | | | | |
| | type 1 motif, 4 | | | | |
| NM_032549 | IMP2 inner mitochondrial | IMMP2L | 5 | 11 | 476-480 |
| | membrane protease-like (S. cerevisiae) | | | | |
| ENSG00000117094 | similar to MST1 (macrophage | ENSG00000117094 | 6 | 12 | 471-475 |
| | stimulating 1 (hepatocyte | | | | |
| | growth factor-like)) | | | | |

### Example 2

### USP21, GZMM, USP2, and ADAMTS4 Up-Regulates Amyloid Beta Peptides in HEK293 APPwt Cells

The stimulatory effect of USP21, GZMM, USP2, and ADAMTS4 is confirmed upon re-screening of the viruses with a known titer (viral particles/ml), as determined by quantitative real time PCR. USP21, GZMM, USP2, and ADAMTS4 adenovirus is infected at MOIs ranging from 2 to 1250 and the experiment is performed as described above. In addition, the effect of USP21, GZMM, USP2, and ADAMTS4 on amyloid beta 1-40, 11-42 and 1-y levels are checked under similar conditions as above. The respective ELISAs are performed as described above, except that the following antibodies are used: for the amyloid beta 1-40 ELISA, the capture and detection antibody are respectively JRF/cAbeta40/10 and JRF/AbetaN/25-HRP (obtained from M Mercken, Johnson and Johnson Pharmaceutical Research and Development, B-2340 Beerse, Belgium), for the amyloid beta 11-42 ELISA, the capture and detection antibody are respectively JRF/cAbeta42/26 and JRF/hAb11/1 (obtained from M Mercken, Johnson and Johnson Pharmaceutical Research and Development, B-2340 Beerse, Belgium), while for the amyloid beta 1-y ELISA (y ranges from 24-42) the capture and detection antibodies are JRF/AbetaN/25 and 4G8-HRP, respectively (obtained respectively from M Mercken, Johnson and Johnson Pharmaceutical Research and Development, B-2340 Beerse, Belgium and from Signet, USA). The amyloid beta 1-y ELISA is used for the detection of amyloid peptides with a variable C-terminus (amyloid beta 1-37; 1-38; 1-39; 1-40; 1-42).

### Example 3

### Reduction of the Amyloid Beta Production Via Knock Down of the Expression Levels of Identified Targets

The effect of an antagonist can be mimicked through the use of siRNA based strategies, which result in decreased expression levels of the targeted protein. HEK293 APPwt cells were transfected with a pool of siRNAs (Table 6) targeted against USP21 or GZMM, eGFP, luciferase and BACE1 using Oligofectamine transfection reagent. 24 hours after transfection, medium was refreshed and the cells were allowed to accumulate amyloid beta peptides in the conditioned medium for an additional 24 hours prior to analysis with the Abeta 1-42 ELISA described above.

TABLE 6

sIRNA sequences. The 4 duplexes constitute
1 pool that was used in the experiments.

| | | | | | |
|---|---|---|---|---|---|
| | | | SEQ | | SEQ |
| | Duplex | | ID | | ID |
| Gene | ID | siRNA sense strand | NO: | siRNA antisense strand | NO: |

| | | | | | |
|---|---|---|---|---|---|
| USP21 | 1 | GUACAAAGAUUCCCUCGAAUU | 49 | ′5-P UCGAGGGAAUCUUUGUACUU | 50 |
| | 2 | GAACCUGAGUUAAGUGAUGUU | 51 | ′5-P CAUCACUUAACUCAGGUUCUU | 52 |
| | 3 | GAGCUGUCUUCCAGAAAUAUU | 53 | ′5-P UAUUUCUGGAAGACAGCUCUU | 54 |
| | 4 | GAGCAGCACUCGACCUCUUUU | 55 | ′5-P AAGAGGUCGAGUGCUGCUCUU | 56 |
| GZMM | 1 | GGUCUGCACUGACAUCUUCUU | 57 | ′5-P GAAGAUGUCAGUGCAGACCUU | 58 |
| | 2 | GGUCUCACCUUCCACAUCAUU | 59 | ′5-P GAUGUGGAAGGUGAGACCUU | 60 |
| | 3 | GCCCGUACAUGGCCUCACUUU | 61 | ′5-P AGUGAGGCCAUGUACGGGCUU | 62 |
| | 4 | CGCCUUACGUGUCCUGGAUUU | 63 | ′5-P AUCCAGGACACGUAAGGCGUU | 64 |
| GL2 | 1 | CGUACGCGGAAUACUUCGAUUU | 65 | UCGAAGUAUUCCGCGUACG | 66 |
| eGFP | 1 | GGCUACGUCCAGGAGCGCACC | 67 | ′5-P UGCGCUCCUGGACGUAGCUU | 68 |

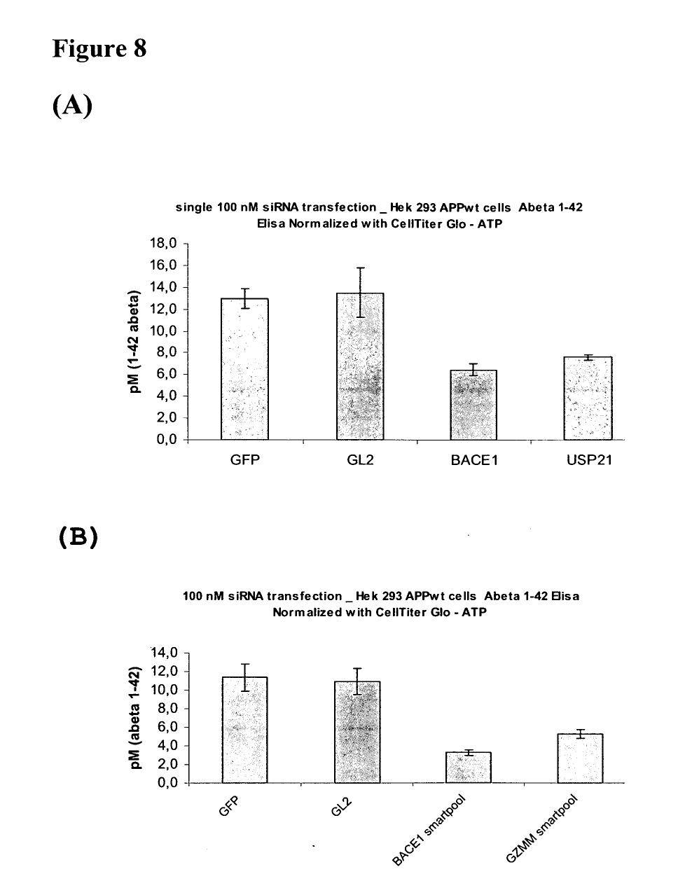

The data clearly show that siRNA targeted against the polypeptides of the invention reduce amyloid beta 1-42 levels compared to the control conditions (FIG. 8: A represents the results with USP21 and B represent the results with GZMM). In conclusion, these data show that the identified polypeptides according to the present invention modulate the levels of secreted amyloid beta.

### Example 4

### USP21, GZMM, USP2, and ADAMTS4 Expression in Human Brain Tissue

Upon identification of a protein protease involved of APP processing, it is essential to evaluate whether the protease is expressed in the tissue and cells of interest. This can be achieved by measuring RNA and/or protein levels. In recent years, RNA levels are being quantified through real time PCR technologies, whereby the RNA is first transcribed to cDNA and then the amplification of the cDNA of interest is monitored during a PCR reaction. The amplification plot and the resulting Ct value are indicators for the amount of RNA present in the sample. To assess whether USP21, GZMM, USP2, and ADAMTS4 cDNA is expressed in the human brain, real time PCR with GAPDH specific primers and specific primers for polynucleotides coding for the USP21, GZMM, USP2, and ADAMTS4 polypeptide (Table 7) is performed on human total brain, human cerebral cortex, and human hippocampal total RNA (BD Biosciences). GAPDH RNA is detected with a Taqman probe, while for the USP21, GZMM, USP2, and ADAMTS4 polynucleotides SybrGreen is used. 40 ng of RNA is transcribed to DNA using the MultiScribe Reverse Transcriptase (50 U/μl) enzyme (Applied BioSystems). The resulting cDNA is amplified with AmpliTaq Gold DNA polymerase (Applied BioSystems) during 40 cycles using an ABI PRISM® 7000 Sequence Detection System.

TABLE 7

Primers used in the quantitative real time PCR analysis
for expression levels of USP21, GZMM, USP2, and ADAMTS4
polynucleotides

| | | | | |
|---|---|---|---|---|
| | | | SEQ ID | |
| Gene | Species | Primer name | NO's | Sequence |

| | | | | |
|---|---|---|---|---|
| USP21 | H. Sapiens | USP21_Hs_For | 33 | CTGCGAAGCTGTGAATCCTACTC |
| | H. Sapiens | USP21_Hs_Rev | 34 | GGCATCCTGCTGGCTGTATC |
| USP2 | H. Sapiens | USP2_Hs_For | 35 | GATACGCACCGCGCTTT |
| | H. Sapiens | USP2_Hs_Rev | 36 | ATGGAGCCCATCCAGAAGAA |
| ADAMTS4 | H. Sapiens | ADAMTS4_Hs_F | 37 | TTTGACACAGCCATTCTGTTTACC |
| | H. Sapiens | ADAMTS4_Hs_R | 38 | GAGCCCATCATCCTCCACAA |
| GZMM | H. Sapiens | GZMM_Hs_For | 39 | ACATGGCCTCACTGCAGAGAA |
| | H. Sapiens | GZMM_Hs_Rev | 40 | GCCGTCAGCACCCACTFTT |
| USP21 | M. Musculus | USP21_Mm_For | 41 | GCAAGATTGTGGACCTGTTTGT |
| | M. Musculus | USP21_Mm_Rev | 42 | CGAAGGTCGTGGAGCGATA |
| USP2 | M. Musculus | USP2_Mm_For | 43 | CCACTAAGAGACCTGGACTTGA |
| | M. Musculus | USP2_Mm_Rev | 44 | GATTGGACACAGCATACAGGTTGT |
| ADAMTS4 | M. Musculus | ADAMTS4_Mm_For | 45 | TCCCATTTCCCGCAGACC |
| | M. Musculus | ADAMTS4_Mm_Rev | 46 | GTCATCTGCTACCACCAGTGT |
| GZMM | M. Musculus | GZMM_Mm_For | 47 | CCCTGCAAGGGTGACTCT |
| | M. Musculus | GZMM Mm Rev | 48 | ACAGGTGGCTTGAAGATGTCTGT |

Total RNA isolated from rat primary neurons and human total brain, cerebral cortex and hippocampal is analyzed, via quantitative real time PCR, for the presence of USP21, GZMM, USP2, and ADAMTS4 cDNA. Table 8 below lists the Ct values for USP21, GZMM, USP2, and ADAMTS4 indicate that USP21, GZMM, USP2, and ADAMTS4 cDNA is detected in all RNA samples.

TABLE 8

Ct

| Gene | Tissue | RT+ | RT− |
|---|---|---|---|
| USP21 | Human Brain | 22.16 | 40 |
| | Human Brain Hippocampus | 22.41 | 40 |
| | Human Brain Cerebral Cortex | 22.56 | 40 |
| USP2 | Human Brain | 22.10 | 32.56 |
| | Human Brain Hippocampus | 22.25 | 34.79 |
| | Human Brain Cerebral Cortex | 21.55 | 32.44 |
| ADAMTS4 | Human Brain | 20.75 | 36.64 |
| | Human Brain Hippocampus | 20.74 | 39.06 |
| | Human Brain Cerebral Cortex | 20.94 | 34.60 |
| GZMM | Human Brain | 27.09 | 32.83 |
| | Human Brain Hippocampus | 28.39 | 33.08 |
| | Human Brain Cerebral Cortex | 28.28 | 32.51 |
| USP21 | Mus Musculus Primary Neurons | 23.08 | 36.14 |
| USP2 | Mus Musculus Primary Neurons | 22.80 | 40 |
| ADAMTS4 | Mus Musculus Primary Neurons | 27.20 | 32.07 |
| GZMM | Mus Musculus Primary Neurons | 29.20 | 40 |

To gain more insight into the specific cellular expression, immuno-histochemistry (protein level) and/or in situ hybridization (RNA level) is carried out on sections from normal and Alzheimer's human brain hippocampal, cortical and subcortical structures, in diseased and normal tissues. These studies measure expression in neurons, microglia cells and astrocytes, and are able to detect differential PROTEASE expression between diseased and healthy tissues.

### Example 5

### Reduction of Amyloid Beta Peptide Levels in Neuronal Cells

Human, mouse or rat primary hippocampal or cortical neurons are transduced with adenoviruses expressing the PROTEASE polypeptides. Amyloid beta levels are determined by ELISA and mass spectrometry analysis. Since rodent APP genes carry a number of mutations in APP compared to the human sequence, a detection antibody recognizing rodent amyloid beta is used (JRF/rAb/2; obtained from M Mercken, Johnson and Johnson Pharmaceutical Research and Development, B-2340 Beerse, Belgium). Alternatively, the human amyloid beta ELISAs (see EXAMPLE 1) is performed on cells co-transduction with human wild type APP or human Swedish mutant APP (which enhances amyloid-beta production) cDNA.

Human primary neurons are purchased from Cellial Technologies, France. Rat primary neuron cultures are prepared from brain of E18-E19-day-old fetal Sprague Dawley rats and mouse primary neuron cultures from E14 (cortical cultures) or E17 (cortical and hippocampal cultures)-day old fetal FVB mice, according to Goslin and Banker (Culturing Nerve cells, second edition, 1998 ISBN 0-262-02438-1). Single cell suspensions are prepared from hippocampus or cortical samples. The number of cells is determined (only taking into account the living cells) and cells are plated on poly-L-lysine-coated plastic 96-well plates in minimal essential medium (MEM) supplemented with 10% horse serum. The cells are seeded at a density between 30,000 and 60,000 cells per well (i.e. about 100,000-200,000 cells/cm², respectively). After 3-4 h, culture medium is replaced by 150 μl serum-free neurobasal medium with B27 supplement (GIBCO BRL). Cytosine arabinoside (5 μM) is added 24 h after plating to prevent non-neuronal (glial) cell proliferation.

Neurons are used at day 5-7 after plating. Before adenoviral transduction, 150 μl conditioned medium of these cultures is transferred to the corresponding wells in an empty 96-well plate and 50 μl of the conditioned medium is returned to the cells. The remaining 100 μl/well is stored at 37° C. and 5% CO₂. Both hippocampal and cortical primary neuron cultures are co-infected with the crude lysate of virus containing the cDNAs of the PROTEASE polypeptides, and human wild type APP or human Swedish mutant APP, at different MOIs, ranging from 100 to 3000. Sixteen to twenty-four hours after transduction, virus is removed and cultures are washed with 100 μl pre-warmed fresh neurobasal medium. After removal of the wash solution, the remaining 100 μl of the stored conditioned medium is transferred to the corresponding cells. From this point on, cells secrete amyloid beta peptide into the conditioned medium and its concentration is determined by either rodent or human amyloid beta 1-42 specific ELISAs (see EXAMPLE 1). The conditioned media are collected 24, 48 and 96 hours after exchanging virus-containing medium by stored conditioned medium.

### Example 6

### Amyloid Beta Peptide Reduction Via Knock Down of PROTEASE Expression

The effect of an antagonist can be mimicked through the use of siRNA-based strategies, which result in decreased expression levels of the targeted protein. Adenoviral mediated siRNA or knock down constructs based upon the sequences shown in Table 9, are constructed as described in WO03/020931.

TABLE 9

Knock-Down (KD) Sequences

| | | | | | | |
|---|---|---|---|---|---|---|
| | | Gen- | | | | |
| SEQ | | Bank | | Knock-Down (KD) | | |
| ID | Gene | Access- | Gene | Sequence | | Pos- |
| No. | Symbol | ion No. | description | (19 and 21-mers) | Oligo name | ition |

| | | | | | | |
|---|---|---|---|---|---|---|
| 481 | USP21 | NM_0124 | Homo | CCATGTTACGACCTCTGCCTC | NM_016572_ | 227 |
| | | 75 | sapiens | | idx227 | |
| | | | ubiquitin | | | |
| | | | specific | | | |
| | | | protease 21 | | | |
| | | | (USP21), | | | |
| | | | transcript | | | |
| | | | variant 1, | | | |
| | | | mRNA. | | | |
| 482 | USP21 | NM_0124 | USP21tv_1 | AACGGCTCAAGAAACTGGAGC | NM_012475_ | 269 |
| | | 75 | mRNA | | idx269 | |
| 483 | USP21 | NM_0124 | USP21tv_1 | TCAAGAAACTGGAGCTGGGAC | NM_016572_ | 275 |
| | | 75 | mRNA | | idx275 | |
| 484 | USP21 | NM_0124 | USP21tv_1 | CCAACAGTGGCTTTGCCTCTC | NM_016572_ | 373 |
| | | 75 | mRNA | | idx373 | |
| 485 | USP21 | NM_0124 | USP21tv_1 | AACAGTGGCTTTGCCTCTCCC | NM_012475_ | 375 |
| | | 75 | mRNA | | idx375 | |
| 486 | USP21 | NM_0124 | USP21tv_1 | CCCATCTCGGACCAACTTAGC | NM_016572_ | 393 |
| | | 75 | mRNA | | idx393 | |
| 487 | USP21 | NM_0124 | USP21tv_1 | CCATCTCGGACCAACTTAGCC | NM_016572_ | 394 |
| | | 75 | mRNA | | idx394 | |
| 488 | USP21 | NM_0124 | USP21tv_1 | TCGGACCAACTTAGCCCGTTC | NM_016572_ | 399 |
| | | 75 | mRNA | | idx399 | |
| 489 | USP21 | NM_0124 | USP21tv_1 | CCACCCACTTTGAGACGTAGC | NM_016572_ | 529 |
| | | 75 | mRNA | | idx529 | |
| 490 | USP21 | NM_0124 | USP21tv_1 | ACCCACTTTGAGACGTAGCAC | NM_012475_ | 531 |
| | | 75 | mRNA | | idx531 | |
| 491 | USP21 | NM_0124 | USP21tv_1 | ACTTCCCATGGCTCCTTCCAC | NM_013919_ | 619 |
| | | 75 | mRNA | | idx552 | |
| 492 | USP21 | NM_0124 | USP21tv_1 | TCCTTCCACATGATATCCGCC | NM_016572_ | 631 |
| | | 75 | mRNA | | idx631 | |
| 493 | USP21 | NM_0124 | USP21tv_1 | CCTTCCACATGATATCCGCCC | NM_016572_ | 632 |
| | | 75 | mRNA | | idx632 | |
| 494 | USP21 | NM_0124 | USP21tv_1 | ACTCTGATGACAAGATGGCTC | NM_012475_ | 671 |
| | | 75 | mRNA | | idx671 | |
| 495 | USP21 | NM_0124 | USP21tv_1 | ACAAGATGGCTCATCACACAC | NM_012475_ | 680 |
| | | 75 | mRNA | | idx680 | |
| 496 | USP21 | NM_0124 | USP21tv_1 | AAGATGGCTCATCACACACTC | NM_012475_ | 682 |
| | | 75 | mRNA | | idx652 | |
| 497 | USP21 | NM_0124 | USP21tv_1 | TCACACACTCCTTCTGGGCTC | NM_016572_ | 693 |
| | | 75 | mRNA | | idx693 | |
| 498 | USP21 | NM_0124 | USP21tv_1 | GCTCTGGTCATGTTGGCCTTC | NM_016572_ | 710 |
| | | 75 | mRNA | | idx710 | |
| 499 | USP21 | NM_0124 | USP21tv_1 | CCTTCGAAACCTGGGAAACAC | NM_016572_ | 726 |
| | | 75 | mRNA | | idx726 | |
| 500 | USP21 | NM_0124 | USP21tv_1 | AACCTGGGAAACACGTGCTTC | NM_012475_ | 733 |
| | | 75 | mRNA | | idx733 | |
| 501 | USP21 | NM_0124 | USP21tv_1 | ACCTGGGAAACACGTGCTTCC | NM_012475_ | 734 |
| | | 75 | mRNA | | idx734 | |
| 502 | USP21 | NM_0124 | USP21tv_1 | AAACACGTGCTTCCTGAATGC | NM_012475_ | 741 |
| | | 75 | mRNA | | idx741 | |
| 503 | USP21 | NM_0124 | USP21tv_1 | GCTTCCTGAATGCTGTGCTGC | NM_016572_ | 749 |
| | | 75 | mRNA | | idx749 | |
| 504 | USP21 | NM_0124 | USP21tv_1 | ACTCGACCTCTTCGGGACTTC | NM_012475_ | 784 |
| | | 75 | mRNA | | idx784 | |
| 505 | USP21 | NM_0124 | USP21tv_1 | TCTGTCTGAGAAGGGACTTCC | NM_016572_ | 803 |
| | | 75 | mRNA | | idx803 | |
| 506 | USP21 | NM_0124 | USP21tv_1 | GCAGATGTGATTGGTGCCCTC | NM_016572_ | 874 |
| | | 75 | mRNA | | idx874 | |
| 507 | USP21 | NM_0124 | USP21tv_1 | ACTCCTGCGAAGCTGTGAATC | NM_012475_ | 905 |
| | | 75 | mRNA | | idx905 | |
| 508 | USP21 | NM_0124 | USP21tv_1 | GCGAAGCTGTGAATCCTACTC | NM_016572_ | 911 |
| | | 75 | mRNA | | idx911 | |
| 509 | USP21 | NM_0124 | USP21tv_1 | GCTGTGAATCCTACTCGATTC | NM_016572_ | 916 |
| | | 75 | mRNA | | idx916 | |
| 510 | USP21 | NM_0124 | USP21tv_1 | CCTACTCGATTCCGAGCTGTC | NM_016572_ | 925 |
| | | 75 | mRNA | | idx925 | |
| 511 | USP21 | NM_0124 | USP21tv_1 | ACTCGATTCCGAGCTGTCTTC | NM_012475_ | 928 |
| | | 75 | mRNA | | idx928 | |
| 512 | USP21 | NM_0124 | USP21tv_1 | ACCGATACTTGCCAATGGTCC | NM_012475_ | 1062 |
| | | 75 | mRNA | | idx1062 | |
| 513 | USP21 | NM_0124 | USP21tv_1 | ACTTGCCAATGGTCCAGTTCC | NM_012475 | 1068 |
| | | 75 | mRNA | | idx1068 | |
| 514 | USP21 | NM_0124 | USP21tv_1 | ACCTAATGTGGAAACGTTACC | NM_012475_ | 1154 |
| | | 75 | mRNA | | idx1154 | |
| 515 | USP21 | NM_0124 | USP21tv_1 | AAGACAGCAAGATTGTGGACC | NM_013919_ | 1184 |
| | | 75 | mRNA | | idx1120 | |
| 516 | USP21 | NM_0124 | USP21tv_1 | AAGTTGTCTCAAGTGCCAGGC | NM_012475_ | 1224 |
| | | 75 | mRNA | | idx1224 | |
| 517 | USP21 | NM_0124 | USP21tv_1 | AAAGCCGGAAGTCCTGTATAC | NM_012475_ | 1573 |
| | | 75 | mRNA | | idx1573 | |
| 518 | USP21 | NM_0124 | USP21tv_1 | AAGCCGGAAGTCCTGTATACC | NM_012475_ | 1574 |
| | | 75 | mRNA | | idx1574 | |
| 519 | USP21 | NM_0124 | USP21tv_1 | ACTATGGCCACTACACAGCCC | NM_012475_ | 1631 |
| | | 75 | mRNA | | idx1631 | |
| 520 | USP21 | NM_0124 | USP21tv_1 | ACAATGACTCTCGTGTCTCCC | NM_012475_ | 1682 |
| | | 75 | mRNA | | idx1682 | |
| 521 | USP21 | NM_0124 | USP21tv_1 | ACCAACTGATGCAGGAGCCAC | NM_012475_ | 1751 |
| | | 75 | mRNA | | idx1751 | |
| 522 | USP21 | NM_0124 | USP21tv_1 | ACACCTCTAAGCTCTGGCACC | NM_012475_ | 1785 |
| | | 75 | mRNA | | idx1785 | |
| 523 | USP21 | NM_0124 | USP21tv_1 | AAGCTCTGGCACCTGTGAAGC | NM_012475_ | 1793 |
| | | 75 | mRNA | | idx1793 | |
| 524 | USP21 | NM_0124 | USP21tv_1 | AATACCCTTCCACCTGGAGGC | NM_012475_ | 1933 |
| | | 75 | mRNA | | idx1933 | |
| 525 | USP21 | NM_0165 | Homo | CCATGTTACGACCTCTGCCTC | NM_016572_ | 227 |
| | | 72 | sapiens | | idx227 | |
| | | | ubiquitin | | | |
| | | | specific | | | |
| | | | protease 21 | | | |
| | | | (USP21), | | | |
| | | | transcript | | | |
| | | | variant 2, | | | |
| | | | mRNA. | | | |
| 526 | USP21 | NM_0165 | USP21tv_2 | AACGGCTCAAGAAACTGGAGC | NM_012475_ | 269 |
| | | 72 | mRNA | | idx269 | |
| 527 | USP21 | NM_0165 | USP21tv_2 | TCAAGAAACTGGAGCTGGGAC | NM_016572_ | 275 |
| | | 72 | mRNA | | idx275 | |
| 528 | USP21 | NM_0165 | USP21tv_2 | CCAACAGTGGCTTTGCCTCTC | NM_016572_ | 373 |
| | | 72 | mRNA | | idx373 | |
| 529 | USP21 | NM_0165 | USP21tv_2 | AACAGTGGCTTTGCCTCTCCC | NM_012475_ | 375 |
| | | 72 | mRNA | | idx375 | |
| 530 | USP21 | NM_0165 | USP21tv_2 | CCCATCTCGGACCAACTTAGC | NM_016572_ | 393 |
| | | 72 | mRNA | | idx393 | |
| 531 | USP21 | NM_0165 | USP21tv_2 | CCATCTCGGACCAACTTAGCC | NM_016572_ | 394 |
| | | 72 | mRNA | | idx394 | |
| 532 | USP21 | NM_0165 | USP21tv_2 | TCGGACCAACTTAGCCCGTTC | NM_016572_ | 399 |
| | | 72 | mRNA | | idx399 | |
| 533 | USP21 | NM_0165 | USP21tv 2 | CCACCCACTTTGAGACGTAGC | NM_016572_ | 529 |
| | | 72 | mRNA | | idx529 | |
| 534 | USP21 | NM_0165 | USP21tv_2 | ACCCACTTTGAGACGTAGCAC | NM_012475_ | 531 |
| | | 72 | mRNA | | idx531 | |
| 535 | USP21 | NM_0165 | USP21tv_2 | ACTTCCCATGGCTCCTTCCAC | NM_013919_ | 619 |
| | | 72 | mRNA | | idx552 | |
| 536 | USP21 | NM_0165 | USP21tv_2 | TCCITCCACATGATATCCGCC | NM_016572_ | 631 |
| | | 72 | mRNA | | idx631 | |
| 537 | USP21 | NM_0165 | USP21tv_2 | CCTTCCACATGATATCCGCCC | NM_016572_ | 632 |
| | | 72 - | mRNA | | idx632 | |
| 538 | USP21 | NM_0165 | USP21tv_2 | ACTCTGATGACAAGATGGCTC | NM_012475_ | 671 |
| | | 72 | mRNA | | idx671 | |
| 539 | USP21 | NM_0165 | USP21tv_2 | ACAAGATGGCTCATCACACAC | NM_012475_ | 680 |
| | | 72 | mRNA | | idx680 | |
| 540 | USP21 | NM_0165 | USP21tv_2 | AAGATGGCTCATCACACACTC | NM_012475_ | 682 |
| | | 72 | mRNA | | idx682 | |
| 541 | USP21 | NM_0165 | USP21tv_2 | TCACACACTCCTTCTGGGCTC | NM_016572_ | 693 |
| | | 72 | mRNA | | idx693 | |
| 542 | USP21 | NM_0165 | USP21tv_2 | GCTCTGGTCATGTTGGCCTTC | NM_016572_ | 710 |
| | | 72 | mRNA | | idx710 | |
| 543 | USP21 | NM_0165 | USP21tv_2 | CCTTCGAAACCTGGGAAACAC | NM_016572_ | 726 |
| | | 72 | mRNA | | idx726 | |
| 544 | USP21 | NM_0165 | USP21tv_2 | AACCTGGGAAACACGTGCTTC | NM_012475_ | 733 |
| | | 72 | mRNA | | idx733 | |
| 545 | USP21 | NM_0165 | USP21tv_2 | ACCTGGGAAACACGTGCTTCC | NM_012475_ | 734 |
| | | 72 | mRNA | | idx734 | |
| 546 | USP21 | NM_0165 | USP21tv_2 | AAACACGTGCTTCCTGAATGC | NM_012475_ | 741 |
| | | 72 | mRNA | | idx741 | |
| 547 | USP21 | NM_0165 | USP21tv_2 | GCTTCCTGAATGCTGTGCTGC | NM_016572_ | 749 |
| | | 72 | mRNA | | idx749 | |
| 548 | USP21 | NM_0165 | USP21tv_2 | ACTCGACCTCTTCGGGACTTC | NM_012475_ | 784 |
| | | 72 | mRNA | | idx784 | |
| 549 | USP21 | NM_0165 | USP21tv_2 | TCTGTCTGAGAAGGGACTTCC | NM_016572_ | 803 |
| | | 72 | mRNA | | idx803 | |
| 550 | USP21 | NM_0165 | USP21tv_2 | GCAGATGTGATTGGTGCCCTC | NM_016572_ | 874 |
| | | 72 | mRNA | | idx874 | |
| 551 | USP21 | NM_0165 | USP21tv_2 | ACTCCTGCGAAGCTGTGAATC | NM_012475_ | 905 |
| | | 72 | mRNA | | idx905 | |
| 552 | USP21 | NM_0165 | USP21tv_2 | GCGAAGCTGTGAATCCTACTC | NM_016572_ | 911 |
| | | 72 | mRNA | | idx911 | |
| 553 | USP21 | NM_0165 | USP21tv_2 | GCTGTGAATCCTACTCGATTC | NM_016572_ | 916 |
| | | 72 | mRNA | | idx9 16 | |
| 554 | USP21 | NM_0165 | USP21tv_2 | CCTACTCGATTCCGAGCTGTC | NM_016572_ | 925 |
| | | 72 | mRNA | | idx925 | |
| 555 | USP21 | NM_0165 | USP21tv_2 | ACTCGATTCCGAGCTGTCTTC | NM_012475_ | 928 |
| | | 72 | mRNA | | idx928 | |
| 556 | USP21 | NM_0165 | USP21tv_2 | ACCGATACTTGCCAATGGTCC | NM_012475_ | 1062 |
| | | 72 | mRNA | | idx1062 | |
| 557 | USP21 | NM_0165 | USP21tv_2 | ACTTGCCAATGGTCCAGTTCC | NM_012475_ | 1068 |
| | | 72 | mRNA | | idx1068 | |
| 558 | USP21 | NM_0165 | USP21tv_2 | ACCTAATGTGGAAACGTTACC | NM_012475_ | 1154 |
| | | 72 | mRNA | | idx1154 | |
| 559 | USP21 | NM_0165 | USP21tv_2 | AAGACAGCAAGATTGTGGACC | NM_013919_ | 1184 |
| | | 72 | mRNA | | idx1120 | |
| 560 | USP21 | NM_0165 | USP21tv_2 | AAGTTGTCTCAAGTGCCAGGC | NM_012475_ | 1224 |
| | | 72 | mRNA | | idx1224 | |
| 561 | USP21 | NM_0165 | USP21tv_2 | ACTATGGCCACTACACAGCCC | NM_012475_ | 1589 |
| | | 72 | mRNA | | idx1631 | |
| 562 | USP21 | NM_0165 | USP21tv_2 | ACAATGACTCTCGTGTCTCCC | NM_012475_ | 1640 |
| | | 72 | mRNA | | idx1682 | |
| 563 | USP21 | NM_0165 | USP21tv_2 | ACCAACTGATGCAGGAGCCAC | NM_012475_ | 1709 |
| | | 72 | mRNA | | idx1751 | |
| 564 | USP21 | NM_0165 | USP21tv_2 | ACACCTCTAAGCTCTGGCACC | NM_012475_ | 1743 |
| | | 72 | mRNA | | idx1785 | |
| 565 | USP21 | NM_0165 | USP21tv_2 | AAGCTCTGGCACCTGTGAAGC | NM_012475_ | 1751 |
| | | 72 | mRNA | | idx1793 | |
| 566 | USP21 | NM_0165 | USP21tv_2 | AATACCCTTCCACCTGGAGGC | NM_012475_ | 1891 |
| | | 72 | mRNA | | idx1933 | |
| 567 | USP21 | NM_0124 | Homo | ATGTTACGACCTCTGCCTC | NM_016572_ | 227 |
| | | 75 | sapiens | | idx227 | |
| | | | ubiquitin | | | |
| | | | specific | | | |
| | | | protease 21 | | | |
| | | | (USP21), | | | |
| | | | transcript | | | |
| | | | variant 1, | | | |
| | | | mRNA. | | | |
| 568 | USP21 | NM_0124 | USP21tv_1 | CGGCTCAAGAAACTGGAGC | NM_012475_ | 269 |
| | | 75 | mRNA | idx269 | | |
| 569 | USP21 | NM_0124 | USP21tv_1 | AAGAAACTGGAGCTGGGAC | NM_016572_ | 275 |
| | | 75 | mRNA | idx275 | | |
| 570 | USP21 | NM_0124 | USP21tv_1 | AACAGTGGCTTTGCCTCTC | NM_016572- | 373 |
| | | 75 | mRNA | | idx373 | |
| 571 | USP21 | NM_0124 | USP21tv_1 | CAGTGGCTITGCCTCTCCC | NM_012475_ | 375 |
| | | 75 | mRNA | | idx375 | |
| 572 | USP21 | NM_0124 | USP21tv_1 | CATCTCGGACCAACTTAGC | NM_016572_ | 393 |
| | | 75 | mRNA | | idx393 | |
| 573 | USP21 | NM_0124 | USP21tv_1 | ATCTCGGACCAACTTAGCC | NM_016572_ | 394 |
| | | 75 | mRNA | | idx394 | |
| 574 | USP21 | NM_0124 | USP21tv_1 | GGACCAACTTAGCCCGTTC | NM_016572_ | 399 |
| | | 75 | mRNA | | idx399 | |
| 575 | USP21 | NM_0124 | USP21tv_1 | ACCCACTTGAGACGTAGC | NM_016572_ | 529 |
| | | 75 | mRNA | | idx529 | |
| 576 | USP21 | NM_0124 | USP21tv_1 | CCACTTTGAGACGTAGCAC | NM_012475_ | 531 |
| | | 75 | mRNA | | idx531 | |
| 577 | USP21 | NM_0124 | USP21tv_1 | TTCCCATGGCTCCTTCCAC | NM_013919_ | 619 |
| | | 75 | mRNA | | idx552 | |
| 578 | USP21 | NM_0124 | USP21tv_1 | CTTCCACATGATATCCGCC | NM_016572_ | 631 |
| | | 75 | mRNA | | idx631 | |
| 579 | USP21 | NM_0124 | USP21tv_1 | TTCCACATGATATCCGCCC | NM_016572_ | 632 |
| | | 75 | mRNA | | idx632 | |
| 580 | USP21 | NM_0124 | USP21tv_1 | TCTGATGACAAGATGGCTC | NM_012475_ | 671 |
| | | 75 | mRNA | | idx671 | |
| 581 | USP21 | NM_0124 | USP21tv_1 | AAGATGGCTCATCACACAC | NM_012475_ | 680 |
| | | 75 | mRNA | | idx680 | |
| 582 | USP21 | NM_0124 | USP21tv_1 | GATGGCTCATCACACACTC | NM_012475_ | 682 |
| | | 75 | mRNA | | idx682 | |
| 583 | USP21 | NM_0124 | USP21tv_1 | ACACACTCCTTCTGGGCTC | NM_016572_ | 693 |
| | | 75 | mRNA | | idx693 | |
| 584 | USP21 | NM_0124 | USP21tv_1 | TCTGGTCATGTTGGCCTTC | NM_016572_ | 710 |
| | | 75 | mRNA | | idx710 | |
| 585 | USP21 | NM_0124 | USP21tv_1 | TTCGAAACCTGGGAAACAC | NM_016572_ | 726 |
| | | 75 | mRNA | | idx726 | |
| 586 | USP21 | NM_0124 | USP21tv_1 | CCTGGGAAACACGTGCTTC | NM_012475_ | 733 |
| | | 75 | mRNA | | idx733 | |
| 587 | USP21 | NM_0124 | USP21tv_1 | CTGGGAAACACGTGCTTCC | NM_012475_ | 734 |
| | | 75 | mRNA | | idx734 | |
| 588 | USP21 | NM_0124 | USP21tv_1 | ACACGTGCTTCCTGAATGC | NM_012475_ | 741 |
| | | 75 | mRNA | | idx741 | |
| 589 | USP21 | NM_0124 | USP21tv_1 | TTCCTGAATGCTGTGCTGC | NM_016572_ | 749 |
| | | 75 | mRNA | | idx749 | |
| 590 | USP21 | NM_0124 | USP21tv_1 | TCGACCTCTTCGGGACTTC | NM_012475_ | 784 |
| | | 75 | mRNA | | idx784 | |
| 591 | USP21 | NM_0124 | USP21tv_1 | TGTCTGAGAAGGGACTTCC | NM_016572_ | 803 |
| | | 75 | mRNA | | idx803 | |
| 592 | USP21 | NM_0124 | USP21tv_1 | AGATGTGATTGGTGCCCTC | NM_016572_ | 874 |
| | | 75 | mRNA | | idx874 | |
| 593 | USP21 | NM_0124 | USP21tv_1 | TCCTGCGAAGCTGTGAATC | NM_012475_ | 905 |
| | | 75 | mRNA | | idx905 | |
| 594 | USP21 | NM_0124 | USP21tv_1 | GAAGCTGTGAATCCTACTC | NM_016572_ | 911 |
| | | 75 | mRNA | | idx911 | |
| 595 | USP21 | NM_0124 | USP21tv_1 | TGTGAATCCTACTCGATTC | NM_016572_ | 916 |
| | | 75 | mRNA | | idx916 | |
| 596 | USP21 | NM_0124 | USP21tv_1 | TACTCGATTCCGAGCTGTC | NM_016572_ | 925 |
| | | 75 | mRNA | | idx925 | |
| 597 | USP21 | NM_0124 | USP21tv_1 | TCGATTCCGAGCTGTCTTC | NM_012475_ | 928 |
| | | 75 | mRNA | | idx928 | |
| 598 | USP21 | NM_0124 | USP21tv_1 | CGATACTTGCCAATGGTCC | NM_012475_ | 1062 |
| | | 75 | mRNA | | idx1062 | |
| 599 | USP21 | NM_0124 | USP21tv_1 | TTGCCAATGGTCCAGTTCC | NM_012475_ | 1068 |
| | | 75 | mRNA | | idx1068 | |
| 600 | USP21 | NM_0124 | USP21tv 1 | CTAATGTGGAAACGTTACC | NM_012475_ | 1154 |
| | | 75 | mRNA | | idx1154 | |
| 601 | USP21 | NM_0124 | USP21tv_1 | GACAGCAAGATTGTGGACC | NM_013919_ | 1184 |
| | | 75 | mRNA | | idx1120 | |
| 602 | USP21 | NM_0124 | USP21tv_1 | GTTGTCTCAAGTGCCAGGC | NM_012475_ | 1224 |
| | | 75 | mRNA | | idx1224 | |
| 603 | USP21 | NM_0124 | USP21tv_1 | AGCCGGAAGTCCTGTATAC | NM_012475_ | 1573 |
| | | 75 | mRNA | | idx1573 | |
| 604 | USP21 | NM_0124 | USP21tv_1 | GCCGGAAGTCCTGTATACC | NM_012475_ | 1574 |
| | | 75 | mRNA | | idx1574 | |
| 605 | USP21 | NM_0124 | USP21tv_1 | TATGGCCACTACACAGCCC | NM_012475_ | 1631 |
| | | 75 | mRNA | | idx1631 | |
| 606 | USP21 | NM_0124 | USP21tv_1 | AATGACTCTCGTGTCTCCC | NM_012475_ | 1682 |
| | | 75 | mRNA | | idx1682 | |
| 607 | USP21 | NM_0124 | USP21tv_1 | CAACTGATGCAGGAGCCAC | NM_012475_ | 1751 |
| | | 75 | mRNA | | idx1751 | |
| 608 | USP21 | NM_0124 | USP21tv_1 | ACCTCTAAGCTCTGGCACC | NM_012475_ | 1785 |
| | | 75 | mRNA | | idx1785 | |
| 609 | USP21 | NM_0124 | USP21tv_1 | GCTCTGGCACCTGTGAAGC | NM_012475_ | 1793 |
| | | 75 | mRNA | | idx1793 | |
| 610 | USP21 | NM_0124 | USP21tv_1 | TACCCTTCCACCTGGAGGC | NM_012475_ | 1933 |
| | | 75 | mRNA | | idx1933 | |
| 611 | USP21 | NM_0165 | Homo | ATGTTACGACCTCTGCCTC | NM_016572_ | 227 |
| | | 72 | sapiens | | idx227 | |
| | | | ubiquitin | | | |
| | | | specific | | | |
| | | | protease 21 | | | |
| | | | (USP21), | | | |
| | | | transcript | | | |
| | | | variant 2, | | | |
| | | | mRNA. | | | |
| 612 | USP21 | NM_0165 | USP21tv_2 | CGGCTCAAGAAACTGGAGC | NM_012475_ | 269 |
| | | 72 | mRNA | | idx269 | |
| 613 | USP21 | NM_0165 | USP21tv_2 | AAGAAACTGGAGCTGGGAC | NM_016572_ | 275 |
| | | 72 | mRNA | | idx275 | |
| 614 | USP21 | NM_0165 | USP21tv_2 | AACAGTGGCTTTGCCTCTC | NM_016572_ | 373 |
| | | 72 | mRNA | | idx373 | |
| 615 | USP21 | NM_0165 | USP21tv_2 | CAGTGGCTTTGCCTCTCCC | NM_012475_ | 375 |
| | | 72 | mRNA | | idx375 | |
| 616 | USP21 | NM_0165 | USP21tv 2 | CATCTCGGACCAACTTAGC | NM_016572_ | 393 |
| | | 72 | mRNA | | idx393 | |
| 617 | USP21 | NM_0165 | USP21tv_2 | ATCTCGGACCAACTTAGCC | NM_016572_ | 394 |
| | | 72 | mRNA | | idx394 | |
| 618 | USP21 | NM_0165 | USP21tv_2 | GGACCAACTTAGCCCGTTC | NM_016572_ | 399 |
| | | 72 | mRNA | | idx399 | |
| 619 | USP21 | NM_0165 | USP21tv_2 | ACCCACT1TGAGACGTAGC | NM_016572_ | 529 |
| | | 72 | mRNA | | idx529 | |
| 620 | USP21 | NM_0165 | USP21tv_2 | CCACTTTGAGACGTAGCAC | NM_012475_ | 531 |
| | | 72 | mRNA | | idx531 | |
| 621 | USP21 | NM_0165 | USP21tv_2 | TTCCCATGGCTCCTTCCAC | NM_013919_ | 619 |
| | | 72 | mRNA | | idx552 | |
| 622 | USP21 | NM_0165 | USP21tv_2 | CTTCCACATGATATCCGCC | NM_016572_ | 631 |
| | | 72 | mRNA | | idx631 | |
| 623 | USP21 | NM_0165 | USP21tv_2 | TTCCACATGATATCCGCCC | NM_016572_ | 632 |
| | | 72 | mRNA | | idx632 | |
| 624 | USP21 | NM_0165 | USP21tv_2 | TCTGATGACAAGATGGGTC | NM_012475_ | 671 |
| | | 72 | miRNA | | idx67l | |
| 625 | USP21 | NM_0165 | USP21tv_2 | AAGATGGCTCATCACACAC | NM_012475_ | 680 |
| | | 72 | mRNA | | idx680 | |
| 626 | USP21 | NM_0165 | USP21tv_2 | GATGGCTCATCACACACTC | NM_012475_ | 682 |
| | | 72 | mRNA | | idx682 | |
| 627 | USP21 | NM_0165 | USP21tv_2 | ACACACTCCTTCTGGGCTC | NM_016572_ | 693 |
| | | 72 | mRNA | | idx693 | |
| 628 | USP21 | NM_0165 | USP21tv_2 | TCTGGTCATGTTGGCCTTC | NM_016572_ | 710 |
| | | 72 | mRNA | | idx710 | |
| 629 | USP21 | NM_0165 | USP21tv_2 | TTCGAAACCTGGGAAACAC | NM_016572_ | 726 |
| | | 72 | mRNA | | idx726 | |
| 630 | USP21 | NM_0165 | USP21tv_2 | CCTGGGAAACACGTGCTTC | NM_012475_ | 733 |
| | | 72 | mRNA | | idx733 | |
| 631 | USP21 | NM_0165 | USP21tv_2 | CTGGGAAACACGTGCTTCC | NM_012475_ | 734 |
| | | 72 | mRNA | | idx734 | |
| 632 | USP21 | NM_0165 | USP21tv_2 | ACACGTGCTTCCTGAATGC | NM_012475_ | 741 |
| | | 72 | mRNA | | idx741 | |
| 633 | USP21 | NM_0165 | USP21tv_2 | TTCCTGAATGCTGTGCTGC | NM_016572_ | 749 |
| | | 72 | mRNA | | idx749 | |
| 634 | USP21 | NM_0165 | USP21tv_2 | TCGACCTCTTCGGGACTTC | NM_012475_ | 784 |
| | | 72 | mRNA | | idx784 | |
| 635 | USP21 | NM_0165 | USP21tv_2 | TGTCTGAGAAGGGACTTCC | NM_016572_ | 803 |
| | | 72 | mRNA | | idx803 | |
| 636 | USP21 | NM_0165 | USP21tv_2 | AGATGTGATTGGTGCCCTC | NM_016572_ | 874 |
| | | 72 | mRNA | | idx874 | |
| 637 | USP21 | NM_0165 | USP21tv_2 | TCCTGCGAAGCTGTGAATC | NM_012475_ | 905 |
| | | 72 | mRNA | | idx905 | |
| 638 | USP21 | NM_0165 | USP21tv_2 | GAAGCTGTGAATCCTACTC | NM_016572_ | 911 |
| | | 72 | mRNA | | idx911 | |
| 639 | USP21 | NM_0165 | USP21tv_2 | TGTGAATCCTACTCGATTC | NM_016572_ | 916 |
| | | 72 | mRNA | | idx916 | |
| 640 | USP21 | NM_0165 | USP21tv_2 | TACTCGATTCCGAGCTGTC | NM_016572_ | 925 |
| | | 72 | mRNA | | idx925 | |
| 641 | USP21 | NM_0165 | USP21tv_2 | TCGATTCCGAGCTGTCTTC | NM_012475_ | 928 |
| | | 72 | mRNA | | idx928 | |
| 642 | USP21 | NM_0165 | USP21tv_2 | CGATACTTGCCAATGGTCC | NM_012475_ | 1062 |
| | | 72 | mRNA | | idx1062 | |
| 643 | USP21 | NM_0165 | USP21tv_2 | TTGCCAATGGTCCAGTTCC | NM_012475_ | 1068 |
| | | 72 | mRNA | | idx1068 | |
| 644 | USP21 | NM_0165 | USP21tv_2 | CTAATGTGGAAACGTTACC | NM_012475_ | 1154 |
| | | 72 | mRNA | | idx1154 | |
| 645 | USP21 | NM_0165 | USP21tv_2 | GACAGCAAGATTGTGGACC | NM_013919_ | 1184 |
| | | 72 | mRNA | | idxl 120 | |
| 646 | USP21 | NM_0165 | USP21tv_2 | GTTGTCTCAAGTGCCAGGC | NM_012475_ | 1224 |
| | | 72 | mRNA | | idx1224 | |
| 647 | USP21 | NM_0165 | USP21tv_2 | TATGGCCACTACACAGCCC | NM_012475_ | 1589 |
| | | 72 | mRNA | | idx1631 | |
| 648 | USP21 | NM_0165 | USP21tv_2 | AATGAGTCTCGTGTCTCCC | NM_012475_ | 1640 |
| | | 72 | mRNA | | idx1682 | |
| 649 | USP21 | NM_0165 | USP21tv_2 | CAACTGATGCAGGAGCCAC | NM_012475_ | 1709 |
| | | 72 | mRNA | | idx1751 | |
| 650 | USP21 | NM_0165 | USP21tv_2 | ACCTCTAAGCTCTGGCACC | NM_012475_ | 1743 |
| | | 72 | mRNA | | idx1785 | |
| 651 | USP21 | NM_0165 | USP21tv_2 | GCTCTGGCACCTGTGAAGC | NM_012475_ | 1751 |
| | | 72 | mRNA | | idx1793 | |
| 652 | USP21 | NM_0165 | USP21tv_2 | TACCCTTCCACCTGGAGGC | NM_012475_ | 1891 |
| | | 72 | mRNA | | idx1933 | |
| 653 | GZMM | NM_0053 | Homo | CTCACTGCAGAGAAATGGCTC | NM_005317_ | 168 |
| | | 17 | sapiens | | idx168 | |
| | | | granzyme M | | | |
| | | | (lymphocyte | | | |
| | | | met-ase 1) | | | |
| | | | (GZMM), | | | |
| | | | mRNA. | | | |
| 654 | GZMM | NM_0053 | GZMM | ACCTTCCACATCAAGGCAGCC | NM_005317 | 313 |
| | | 17 | mRNA | | idx313 | |
| 655 | GZMM | NM_0053 | GZMM | ACATCAAGGCAGCCATCCAGC | NM_005317 | 320 |
| | | 17 | mRNA | | idx320 | |
| 656 | GZMM | NM_0053 | GZMM | GACACCCGCATGTGTAACAAC | NM_005317 | 559 |
| | | 17 | mRNA | | idx559 | |
| 657 | GZMM | NM_0053 | GZMM | ACCCGCATGTGTAACAACAGC | NM_005317 | 562 |
| | | 17 | mRNA | | idx562 | |
| 658 | GZMM | NM_0053 | GZMM | GCACTGACATCTTCAAGCCTC | NM_005317 | 734 |
| | | 17 | mRNA | | idx734 | |
| 659 | GZMM | NM_0053 | GZMM | ACTGACATCTTCAAGCCTCCC | NM_005317 | 736 |
| | | 17 | mRNA | | idx736 | |
| 660 | GZMM | NM_0053 | GZMM | ACAGGGAGGGACCAATAAATC | NM_005317_ | 910 |
| | | 17 | mRNA | | idx910 | |
| 661 | GZMM | NM_0053 | GZMM | CACTGCAGAGAAATGGCTC | NM_005317_ | 168 |
| | | 17 | mRNA | | idx168 | |
| 662 | GZMM | NM_0053 | GZMM | CTTCCACATCAAGGCAGCC | NM_005317_ | 313 |
| | | 17 | mRNA | | idx313 | |
| 663 | GZMM | NM_0053 | GZMM | ATCAAGGCAGCCATCCAGC | NM_005317_ | 320 |
| | | 17 | mRNA | | idx320 | |
| 664 | GZMM | NM_0053 | GZMM | CACCCGCATGTGTAACAAC | NM_005317_ | 559 |
| | | 17 | mRNA | | idx559 | |
| 665 | GZMM | NM_0053 | GZMM | CCGCATGTGTAACAACAGC | NM_005317_ | 562 |
| | | 17 | mRNA | | idx562 | |
| 666 | GZMM | NM_0053 | GZMM | ACTGACATCTTCAAGCCTC | NM_005317_ | 734 |
| | | 17 | mRNA | | idx734 | |
| 667 | GZMM | NM_0053 | GZMM | TGACATCTTCAAGCCTCCC | NM_005317_ | 736 |
| | | 17 | mRNA | | idx736 | |
| 668 | GZMM | NM_0053 | GZMM | AGGGAGGGACCAATAAATC | NM_005317_ | 910 |
| | | 17 | mRNA | | idx910 | |
| 669 | USP2 | NM_0042 | Homo | AAACTTGGTTTCAAGCCGGTC | NM_004205_ | 366 |
| | | 05 | sapiens | | idx366 | |
| | | | ubiquitin | | | |
| | | | specific | | | |
| | | | protease 2 | | | |
| | | | (USP2), | | | |
| | | | transcript | | | |
| | | | variant 1, | | | |
| | | | mRNA. | | | |
| 670 | USP2 | NM_0042 | USP2tv_1 | AACTTGGTTTCAAGCCGGTCC | NM_004205_ | 367 |
| | 05 | mRNA | | idx367 | | |
| 671 | USP2 | NM_0042 | USP2tv_1 | ACTTGGTTTCAAGCCGGTCCC | NM_004205_ | 368 |
| | 05 | mRNA | | idx368 | | |
| 672 | USP2 | NM_0042 | USP2tv_1 | ACCAACAACTGCCTCAGCTAC | NM_004205_ | 576 |
| | 05 | mRNA | | idx576 | | |
| 673 | USP2 | NM_0042 | USP2tv_1 | ACAACTGCCTCAGCTACCTGC | NM_004205_ | 580 |
| | 05 | mRNA | | idx580 | | |
| 674 | USP2 | NM_0042 | USP2tv_1 | ACCCTAACCCAGAAGCTGGAC | NM_004205_ | 627 |
| | 05 | mRNA | | idx627 | | |
| 675 | USP2 | NM_0042 | USP2tv_1 | AAGCTGGACAGCCAATCAGAC | NM_004205_ | 639 |
| | 05 | mRNA | | idx639 | | |
| 676 | USP2 | NM_0042 | USP2tv_1 | ACAGCCAGCTGCCCTGAATAC | NM_004205_ | 786 |
| | 05 | mRNA | | idx786 | | |
| 677 | USP2 | NM_0042 | USP2tv_1 | ACTACCTGGAGAACTATGGTC | NM_004205_ | 814 |
| | 05 | mRNA | | idx814 | | |
| 678 | USP2 | NM_0042 | USP2tv_1 | AAATCATCAGCCCAACCTACC | NM_004205_ | 889 |
| | 05 | mRNA | | idx889 | | |
| 679 | USP2 | NM_0042 | USP2tv_1 | AACCTTGGGAACACGTGCTTC | NM_004205_ | 1035 |
| | 05 | mRNA | | idx1035 | | |
| 680 | USP2 | NM_0042 | USP2tv_1 | ACTCGGGAGTTGAGAGATTAC | NM_004205_ | 1086 |
| | 05 | mRNA | | idx1086 | | |
| 681 | USP2 | NM_0042 | USP2tv_1 | AAGACCCAGATCCAGAGATAC | NM_004205_ | 1242 |
| | 05 | mRNA | | idx1242 | | |
| 682 | USP2 | NM_0042 | USP2tv_1 | ACGAGGTGAACCGAGTGACAC | NM_004205_ | 1336 |
| | 05 | mRNA | | idx1336 | | |
| 683 | USP2 | NM_0042 | USP2tv_1 | ACACTGAGACCTAAGTCCAAC | NM_004205_ | 1353 |
| | 05 | mRNA | | idx1353 | | |
| 684 | USP2 | NM_0042 | USP2tv_1 | ACTGAGACCTAAGTCCAACCC | NM_004205_ | 1355 |
| | 05 | mRNA | | idx1355 | | |
| 685 | USP2 | NM_0042 | USP2tv_1 | AAGTCCAACCCTGAGAACCTC | NM_004205_ | 1365 |
| | 05 | mRNA | | idx1365 | | |
| 686 | USP2 | NM_0042 | USP2tv_1 | ACCCTGAGAACCTCGATCATC | NM_004205_ | 1372 |
| | 05 | mRNA | | idx1372 | | |
| 687 | USP2 | NM_0042 | USP2tv_1 | AAAGGGCTCGCTGACGTGTAC | NM_004205_ | 1481 |
| | 05 | mRNA | | idx1481 | | |
| 688 | USP2 | NM_0042 | USP2tv_1 | ACGTGTACAGATTGTGGTFAC | NM_004205_ | 1494 |
| | 05 | mRNA | | idx1494 | | |
| 689 | USP2 | NM_0042 | USP2tv_1 | ACTGTTCTACGGTCTTCGACC | NM_004205_ | 1513 |
| | 05 | mRNA | | idx1513 | | |
| 690 | USP2 | NM_0042 | USP2tv_1 | AAGCCAACATGCTGTCGCTGC | NM_004205_ | 1641 |
| | 05 | mRNA | | idx1641 | | |
| 691 | USP2 | NM_0042 | USP2tv_1 | AAGTTCTCCATCCAGAGGTTC | NM_004205_ | 1686 |
| | 05 | mRNA | | idx1686 | | |
| 692 | USP2 | NM_0042 | USP2tv_1 | AACACCAACCATGCTGTTTAC | NM_004205_ | 1827 |
| | 05 | mRNA | | idx1827 | | |
| 693 | USP2 | NM_0042 | USP2tv_1 | ACCAACCATGCTGTTTACAAC | NM_004205_ | 1830 |
| | 05 | mRNA | | idx1830 | | |
| 694 | USP2 | NM_0042 | USP2tv_1 | ACCTGTACGCTGTGTCCAATC | NM_004205_ | 1849 |
| | 05 | mRNA | | idx1849 | | |
| 695 | USP2 | NM_0042 | USP2tv_1 | ACAGGAGAATGGCACACTTTC | NM_004205_ | 1923 |
| | 05 | mRNA | | idx1923 | | |
| 696 | USP2 | NM_0042 | USP2tv_1 | ACTTTCAACGACTCCAGCGTC | NM_004205_ | 1938 |
| | 05 | mRNA | | idx1938 | | |
| 697 | USP2 | NM_0042 | USP2tv_1 | ACAACAACACACAAACCTGAC | NM_004205_ | 2124 |
| | | 05 | mRNA | | idx2124 | |
| 698 | USP2 | NM_0042 | USP2tv_2 | ACAAACCTGAAGCTGCCGAGC | NM_004205_ | 2154 |
| | | 05 | mRNA | | idx2154 | |
| 699 | USP2 | NM_ | USP2tv_2 | AACCTTGGGAACACGTGCTCC | NM_004205_ | 371 |
| | | 171997 | mRNA | | idx1035 | |
| 700 | USP2 | NM_ | USP2tv_2 | ACTCGGGAGTTGAGAGATTAC | NM_004205_ | 422 |
| | | 171997 | mRNA | | idx1086 | |
| 701 | USP2 | NM_ | USP2tv_2 | AAGACCCAGATCCAGAGATAC | NM_004205_ | 578 |
| | | 171997 | mRNA | | idx1242 | |
| 702 | USP2 | NM_ | USP2tv_2 | ACGAGGTGAACCGAGTGACAC | NM_004205_ | 672 |
| | | 171997 | mRNA | | idx1336 | |
| 703 | USP2 | NM_ | USP2tv_2 | ACACTGAGACCTAAGTCCAAC | NM_004205_ | 689 |
| | | 171997 | mRNA | | idx1353 | |
| 704 | USP2 | NM_ | USP2tv_2 | ACTGAGACCTAAGTCCAACCC | NM_004205_ | 691 |
| | | 171997 | mRNA | | idx1355 | |
| 705 | USP2 | NM_ | USP2tv_2 | AAGTCCAACCCTGAGAACCTC | NM_004205_ | 701 |
| | | 171997 | mRNA | | idx1365 | |
| 706 | USP2 | NM_ | USP2tv_2 | ACCCTGAGAACCICGATCATC | NM_004205_ | 708 |
| | | 171997 | mRNA | | idx1372 | |
| 707 | USP2 | NM_ | USP2tv_2 | ACGTGTACAGATTGTGGTTAC | NM_004205_ | 830 |
| | | 171997 | mRNA | | idx1494 | |
| 708 | USP2 | NM_ | USP2tv_2 | ACTGTTCTACGGTCTTCGACC | NM_004205_ | 849 |
| | | 171997 | mRNA | | idx1513 | |
| 709 | USP2 | NM_ | USP2tv_2 | AAGCCAACATGCTGTCGCTGC | NM_004205_ | 977 |
| | | 171997 | mRNA | | idx1641 | |
| 710 | USP2 | NM_ | USP2tv_2 | AAGTTCTCCATCCAGAGGTTC | NM_004205_ | 1022 |
| | | 171997 | mRNA | | idx1686 | |
| 711 | USP2 | NM_ | USP2tv_2 | AACACCAACCATGCTGTTTAC | NM_004205_ | 1163 |
| | | 171997 | mRNA | | idx1827 | |
| 712 | USP2 | NM_ | USP2tv_2 | ACCAACCATGCTGTTTACAAC | NM_004205_ | 1166 |
| | | 171997 | mRNA | | idx1830 | |
| 713 | USP2 | NM_ | USP2tv_2 | ACCTGTACGCTGTGTCCAATC | NM_004205_ | 1185 |
| | | 171997 | mRNA | | idx1849 | |
| 714 | USP2 | NM_ | USP2tv_2 | ACAGGAGAATGGCACACTTTC | NM_004205_ | 1259 |
| | | 171997 | mRNA | | idx1923 | |
| 715 | USP2 | NM_ | USP2tv_2 | ACTTTCAACGACTCCAGCGTC | NM_004205_ | 1274 |
| | | 171997 | mRNA | | idx1938 | |
| 716 | USP2 | NM_0042 | USP2tv_1 | ACTTGGTTTCAAGCCGGTC | NM_004205_ | 366 |
| | | 05 | mRNA | | idx366 | |
| 717 | USP2 | NM_0042 | USP2tv_1 | CTTGGTTTCAAGCCGGTCC | NM_004205_ | 367 |
| | | 05 | mRNA | | idx367 | |
| 718 | USP2 | NM_0042 | USP2tv_1 | TTGGTTTCAAGCCGGTCCC | NM_004205_ | 368 |
| | | 05 | mRNA | | idx368 | |
| 719 | USP2 | NM_0042 | USP2tv_1 | CAACAACTGCCTCAGCTAC | NM_004205_ | 576 |
| | | 05 | mRNA | | idx576 | |
| 720 | USP2 | NM_0042 | USP2tv_1 | AACTGCCTCAGCTACCTGC | NM_004205_ | 580 |
| | | 05 | mRNA | | idx580 | |
| 721 | USP2 | NM_0042 | USP2tv_1 | CCTAACCCAGAAGCTGGAC | NM_004205_ | 627 |
| | | 05 | mRNA | | idx627 | |
| 722 | USP2 | NM_0042 | USP2tv_1 | GCTGGACAGCCAATCAGAC | NM_004205_ | 639 |
| | | 05 | mRNA | | idx639 | |
| 723 | USP2 | NM_0042 | USP2tv_1 | AGCCAGCTGCCCTGAATAC | NM_004205_ | 786 |
| | | 05 | mRNA | | idx786 | |
| 724 | USP2 | NM_0042 | USP2tv_1 | TACCTGGAGAACTATGGTC | NM_004205_ | 814 |
| | | 05 | mRNA | | idx814 | |
| 725 | USP2 | NM_0042 | USP2tv_1 | ATCATCAGCCCAACCTACC | NM_004205_ | 889 |
| | | 05 | mRNA | | idx889 | |
| 726 | USP2 | NM_0042 | USP2tv_1 | CCTTGGGAACACGTGCTTC | NM_004205_ | 1035 |
| | | 05 | mRNA | | idx1035 | |
| 727 | USP2 | NM_0042 | USP2tv_1 | TCGGGAGTTGAGAGATTAC | NM_004205_ | 1086 |
| | | 05 | mRNA | | idx1086 | |
| 728 | USP2 | NM_0042 | USP2tv_1 | GACCCAGATCCAGAGATAC | NM_004205_ | 1242 |
| | | 05 | mRNA | | idx1242 | |
| 729 | USP2 | NM_0042 | USP2tv_1 | GAGGTGAACCGAGTGACAC | NM_004205_ | 1336 |
| | | 05 | mRNA | | idx1336 | |
| 730 | USP2 | NM_0042 | USP2tv_1 | ACTGAGACCTAAGTCCAAC | NM_004205_ | 1353 |
| | | 05 | mRNA | | idx1353 | |
| 731 | USP2 | NM_0042 | USP2tv_1 | TGAGACCTAAGTCCAACCC | NM_004205_ | 1355 |
| | | 05 | mRNA | | idx1355 | |
| 732 | USP2 | NM_0042 | USP2tv_1 | GTCCAACCCTGAGAACCTC | NM_004205_ | 1365 |
| | | 05 | mRNA | | idx1365 | |
| 733 | USP2 | NM_0042 | USP2tv_1 | CCTGAGAACCTCGATCATC | NM_004205_ | 1372 |
| | | 05 | mRNA | | idx1372 | |
| 734 | USP2 | NM_0042 | USP2tv_1 | AGGGCTCGCTGACGTGTAC | NM_004205_ | 1481 |
| | | 05 | mRNA | | idx1481 | |
| 735 | USP2 | NM_0042 | USP2tv_1 | GTGTACAGATTGTGGTTAC | NM_004205_ | 1494 |
| | | 05 | mRNA | | idx1494 | |
| 736 | USP2 | NM_0042 | USP2tv_1 | TGTTCTACGGTCTTCGACC | NM_004205_ | 1513 |
| | | 05 | mRNA | | idx1513 | |
| 737 | USP2 | NM_0042 | USP2tv_1 | GCCAACATGCTGTCGCTGC | NM_004205_ | 1641 |
| | | 05 | mRNA | | idx1641 | |
| 738 | USP2 | NM_0042 | USP2tv_1 | GTTCTCCATCCAGAGGTTC | NM_004205_ | 1686 |
| | | 05 | mRNA | | idx1686 | |
| 739 | USP2 | NM_0042 | USP2tv_1 | CACCAACCATGCTGTITAC | NM_004205_ | 1827 |
| | | 05 | mRNA | | idx1827 | |
| 740 | USP2 | NM_0042 | USP2tv_1 | CAACCATGCTGTTTACAAC | NM_004205_ | 1830 |
| | | 05 | mRNA | | idx1830 | |
| 741 | USP2 | NM_0042 | USP2tv_1 | CTGTACGCTGTGTCCAATC | NM_004205_ | 1849 |
| | | 05 | mRNA | | idx1849 | |
| 742 | USP2 | NM_0042 | USP2tv_1 | AGGAGAATGGCACACTTTC | NM_004205_ | 1923 |
| | | 05 | mRNA | | idx1923 | |
| 743 | USP2 | NM_0042 | USP2tv_1 | TTTCAACGACTCCAGCGTC | NM_004205_ | 1938 |
| | | 05 | mRNA | | idx1938 | |
| 744 | USP2 | NM_0042 | USP2tv_1 | AACAACACACAAACCTGAC | NM_004205_ | 2124 |
| | | 05 | mRNA | | idx2124 | |
| 745 | USP2 | NM_0042 | USP2tv_1 | AAACCTGAAGCTGCCGAGC | NM_004205_ | 2154 |
| | | 05 | mRNA | | idx2154 | |
| 746 | USP2 | NM_ | USP2tv_2 | CCTTGGGAACACGTGCTTC | NM_004205_ | 371 |
| | | 171997 | mRNA | | idx1035 | |
| 747 | USP2 | NM_ | USP2tv_2 | TCGGGAGTTGAGAGATTAC | NM_004205_ | 422 |
| | | 171997 | mRNA | | idx1086 | |
| 748 | USP2 | NM_ | USP2tv_2 | GACCCAGATCCAGAGATAC | NM_004205_ | 578 |
| | | 171997 | mRNA | | idx1242 | |
| 749 | USP2 | NM_ | USP2tv_2 | GAGGTGAACCGAGTGACAC | NM_004205_ | 672 |
| | | 171997 | mRNA | | idx1336 | |
| 750 | USP2 | NM_ | USP2tv_2 | ACTGAGACCTAAGTCCAAC | NM_004205_ | 689 |
| | | 171997 | mRNA | | idx1353 | |
| 751 | USP2 | NM_ | USP2tv_2 | TGAGACCTAAGTCCAACCC | NM_004205_ | 691 |
| | | 171997 | mRNA | | idx1355 | |
| 752 | USP2 | NM_ | USP2tv_2 | GTCCAACCCTGAGAACCTC | NM_004205_ | 701 |
| | | 171997 | mRNA | | idx1365 | |
| 753 | USP2 | NM_ | USP2tv_2 | CCTGAGAACCTCGATCATC | NM_004205_ | 708 |
| | | 171997 | mRNA | | idx1372 | |
| 754 | USP2 | NM_ | USP2tv_2 | GTGTACAGATTGTGGTTAC | NM_004205_ | 830 |
| | | 171997 | mRNA | | idx1494 | |
| 755 | USP2 | NM_ | USP2tv_2 | TGTTCTACGGTCTTCGACC | NM_004205_ | 849 |
| | | 171997 | mRNA | | idx1513 | |
| 756 | USP2 | NM_ | USP2tv_2 | GCCAACATGCTGTCGCTGC | NM_004205_ | 977 |
| | | 171997 | mRNA | | idx1641 | |
| 757 | USP2 | NM_ | USP2tv_2 | GTTCTCCATCCAGAGGTTC | NM_004205_ | 1022 |
| | | 171997 | mRNA | | idx1686 | |
| 758 | USP2 | NM_ | USP2tv_2 | CACCAACCATGCTGTTTAC | NM_004205_ | 1163 |
| | | 171997 | mRNA | | idx1827 | |
| 759 | USP2 | NM_ | USP2tv_2 | CAACCATGCTGTTTACAAC | NM_004205_ | 1166 |
| | | 171997 | mRNA | | idx1830 | |
| 760 | USP2 | NM_ | USP2tv_2 | CTGTACGCTGTGTCCAATC | NM_004205_ | 1185 |
| | | 171997 | mRNA | | idx1849 | |
| 761 | USP2 | NM_ | USP2tv_2 | AGGAGAATGGCACACTTTC | NM_004205_ | 1259 |
| | | 171997 | mRNA | | idx1923 | |
| 762 | USP2 | NM_ | USP2tv_2 | TTTCAACGACTCCAGCGTC | NM_004205_ | 1274 |
| | | 171997 | mRNA | | idx1938 | |
| 763 | ADAMTS4 | NM_0050 | Homo | ACTAGAGCTGGAGCAGGACTC | NM_005099_ | 706 |
| | | 99 | sapiens a | | idx685 | |
| | | | disintegrin- | | | |
| | | | like and | | | |
| | | | metallopro- | | | |
| | | | tease | | | |
| | | | (reprolysin | | | |
| | | | type) with | | | |
| | | | thrombos- | | | |
| | | | pondin type | | | |
| | | | 1 motif, 4 | | | |
| | | | (ADAMTS4), | | | |
| | | | mRNA. | | | |
| 764 | ADAMTS4 | NM_0050 | ADAMTS4 | ACCTACCTGACTGGCACCATC | NM_005099_ | 803 |
| | | 99 | mRNA | | idx782 | |
| 765 | ADAMTS4 | NM_0050 | ADAMTS4 | ACATCCTACGCCGGAAGAGTC | NM_005099_ | 963 |
| | | 99 | mRNA | | idx942 | |
| 766 | ADAMTS4 | NM_0050 | ADAMTS4 | AAGAGCCAAGCGCTTTGCTTC | NM_005099 | 1051 |
| | | 99 | mRNA | | idx1030 | |
| 767 | ADAMTS4 | NM_0050 | ADAMTS4 | ACTGAGTAGATTTGTGGAGAC | NM_005099_ | 1072 |
| | | 99 | mRNA | | idx1051 | |
| 768 | ADAMTS4 | NM_0050 | ADAMTS4 | ACACTGGTGGTGGCAGATGAC | NM_005099_ | 1091 |
| | | 99 | mRNA | | idx1070 | |
| 769 | ADAMTS4 | NM_0050 | ADAMTS4 | AACAGTGATGGCAGCAGCAGC | NM_005099_ | 1156 |
| | | 99 | mRNA | | idx1135 | |
| 770 | ADAMTS4 | NM_0050 | ADAMTS4 | AAGGCCTTCAAGCACCCAAGC | NM_005099_ | 1178 |
| | | 99 | mRNA | | idx1157 | |
| 771 | ADAMTS4 | NM_0050 | ADAMTS4 | ACCACTTTGACACAGCCATTC | NM_005099_ | 1350 |
| | | 99 | mRNA | | idx1329 | |
| 772 | ADAMTS4 | NM_0050 | ADAMTS4 | ACACAGCCATTCTGTTTACCC | NM_005099_ | 1359 |
| | | 99 | mRNA | | idx1338 | |
| 773 | ADAMTS4 | NM_0050 | ADAMTS4 | AACATGCTCCATGACAACTCC | NM_005099_ | 1529 |
| | | 99 | mRNA | | idx1518 | |
| 774 | ADAMTS4 | NM_0050 | ADAMTS4 | ACTGACTTCCTGGACAATGGC | NM_005099_ | 1664 |
| | | 99 | mRNA | | idx1643 | |
| 775 | ADAMTS4 | NM_0050 | ADAMTS4 | ACAATGGCTATGGGCACTGTC | NM_005099_ | 1677 |
| | | 99 | mRNA | | idx1656 | |
| 776 | ADAMTS4 | NM_0050 | ADAMTS4 | AATGGCTATGGGCACTGTCTC | NM_005099_ | 1679 |
| | | 99 | mRNA | | idx1658 | |
| 777 | ADAMTS4 | NM_0050 | ADAMTS4 | ACAAACCAGAGGCTCCATTGC | NM_005099_ | 1704 |
| | | 99 | mRNA | | idx1683 | |
| 778 | ADAMTS4 | NM_0050 | ADAMTS4 | AACCAGAGGCTCCATTGCATC | NM_005099_ | 1707 |
| | | 99 | mRNA | | idx1686 | |
| 779 | ADAMTS4 | NM_0050 | ADAMTS4 | AAGGACTATGATGCTGACCGC | NM_005099_ | 1748 |
| | | 99 | mRNA | | idx1727 | |
| 780 | ADAMTS4 | NM_0050 | ADAMTS4 | ACTCACGCCATTGTCCACAGC | NM_005099_ | 1794 |
| | | 99 | mRNA | | idx1773 | |
| 781 | ADAMTS4 | NM_0050 | ADAMTS4 | AATATTCCACAGGCTGGTGGC | NM_005099_ | 1973 |
| | | 99 | mRNA | | idx1952 | |
| 782 | ADAMTS4 | NM_0050 | ADAMTS4 | ACCGACCTCTTCAAGAGCTTC | NM_005099_ | 2207 |
| | | 99 | mRNA | | idx2186 | |
| 783 | ADAMTS4 | NM_0050 | ADAMTS4 | ACCAGTGCAAACTCACCTGCC | NM_005099_ | 2277 |
| | | 99 | mRNA | | idx2256 | |
| 784 | ADAMTS4 | NM_0050 | ADAMTS4 | ACTACTATGTGCTGGAGCCAC | NM_005099_ | 2316 |
| | | 99 | mRNA | | idx2295 | |
| 785 | ADAMTS4 | NM_0050 | ADAMTS4 | ACGGTTCTGGTTGCAGCAAGC | NM_005099_ | 2469 |
| | | 99 | mRNA | | idx2448 | |
| 786 | ADAMTS4 | NM_0050 | ADAMTS4 | ACAACAATGTGGTCACTATCC | NM_005099_ | 2523 |
| | | 99 | mRNA | | idx2502 | |
| 787 | ADAMTS4 | NM_0050 | ADAMTS4 | AATACACGCTGATGCCCTCCC | NM_005099_ | 2649 |
| | | 99 | mRNA | | idx2628 | |
| 788 | ADAMTS4 | NM_0050 | ADAMTS4 | AAGTCCTAGTGGCTGGCAACC | NM_005099_ | 2778 |
| | | 99 | mRNA | | idx2757 | |
| 789 | ADAMTS4 | NM_0050 | ADAMTS4 | ACACGCCTCCGATACAGCTTC | NM_005099_ | 2807 |
| | | 99 | mRNA | | idx2786 | |
| 790 | ADAMTS4 | NM_0050 | ADAMTS4 | AAATAACCTCACTATCCCGGC | NM_005099_ | 2936 |
| | | 99 | mRNA | | idx2915 | |
| 791 | ADAMTS4 | NM_0050 | ADAMTS4 | ACAGCCCTCCATCTAAACTGC | NM_005099_ | 3158 |
| | | 99 | mRNA | | idx3137 | |
| 792 | ADAMTS4 | NM_0050 | ADAMTS4 | ACAACCTGTTCTGCTTTCCTC | NM_005099_ | 3437 |
| | | 99 | mRNA | | idx3418 | |
| 793 | ADAMTS4 | NM_0050 | ADAMTS4 | ACCTGTTCTGCTTTCCTCTTC | NM_005099_ | 3440 |
| | | 99 | mRNA | | idx3421 | |
| 794 | ADAMTS4 | NM_0050 | ADAMTS4 | AAAGTCAAGGGTAGGGTGGGC | NM_005099_ | 3486 |
| | | 99 | mRNA | | idx3467 | |
| 795 | ADAMTS4 | NM_0050 | ADAMTS4 | ACAGAATCTCGCTCTGTCGCC | NM_005099_ | 3570 |
| | | 99 | mRNA | | idx3551 | |
| 796 | ADAMTS4 | NM_0050 | ADAMTS4 | AATGGCACAATCTCGGCTCAC | NM_005099_ | 3604 |
| | | 99 | mRNA | | idx3585 | |
| 797 | ADAMTS4 | NM_0050 | ADAMTS4 | ACAATCTCGGCTCACTGCATC | NM_005099_ | 3610 |
| | | 99 | mRNA | | idx3591 | |
| 798 | ADAMTS4 | NM_0050 | ADAMTS4 | AATCACTTGAACCCGGGAGGC | XM_ | 3633 |
| | | 99 | mRNA | | 050147_ | |
| | | | | | idx3544 | |
| 799 | ADAMTS4 | NM_0050 | ADAMTS4 | AAGTGATTCTCATGCCTCAGC | NM_005099_ | 3648 |
| | | 99 | mRNA | | idx3629 | |
| 800 | ADAMTS4 | NM_0050 | ADAMTS4 | AATCCCAGCTACTCAGGAGGC | NM_013276_ | 3665 |
| | | 99 | mRNA | | idx3070 | |
| 801 | ADAMTS4 | NM_0050 | ADAMTS4 | AATCCCAGCTACTCAGGAGGC | NM_014395_ | 3665 |
| | | 99 | mRNA | | idx2606 | |
| 802 | ADAMTS4 | NM_0050 | ADAMTS4 | AAAGTAGCTGGGATTACAGGC | NM_016225_ | 3673 |
| | | 99 | mRNA | | idx1419 | |
| 803 | ADAMTS4 | NM_0050 | ADAMTS4 | ACAGAGTCTCGCTATTGTCAC | NM_005099_ | 3739 |
| | | 99 | mRNA | | idx3720 | |
| 804 | ADAMTS4 | NM_0050 | ADAMTS4 | ACCTGGGTTCCAGCAATTCTC | NM_005099_ | 3798 |
| | | 99 | mRNA | | idx3779 | |
| 805 | ADAMTS4 | NM_0050 | ADAMTS4 | AAGCAATTCTCCTGCCTCAGC | NM_007181_ | 3808 |
| | | 99 | mRNA | | idx2505 | |
| 806 | ADAMTS4 | NM_0050 | ADAMTS4 | AACTCCTGACCTTAGGTGATC | NM_005099_ | 3930 |
| | | 99 | mRNA | | idx3911 | |
| 807 | ADAMTS4 | NM_0050 | ADAMTS4 | ACTCCTGACCTTAGGTGATCC | NM_005099_ | 3931 |
| | | 99 | mRNA | | idx3912 | |
| 808 | ADAMTS4 | NM_0050 | ADAMTS4 | TCACGCCTGTAATCCCAGCAC | ENSG | 3970 |
| | | 99 | mRNA | | 00000116032_ | |
| | | | | | idx3384 | |
| 809 | ADAMTS4 | NM_0050 | ADAMTS4 | ACTGGGATTACAGGCGTGAGC | NM_024628_ | 3974 |
| | | 99 | mRNA | | idx2003 | |
| 810 | ADAMTS4 | NM_0050 | ADAMTS4 | ACGGTGAAACCCTGTCTCTAC | ENSG | 4033 |
| | | 99 | mRNA | | 00000115257_ | |
| | | | | | idx1012 | |
| 811 | ADAMTS4 | NM_0050 | ADAMTS4 | AACATGGTGAAACCCTGTCTC | NM_022973_ | 4036 |
| | | 99 | mRNA | | idx3029 | |
| 812 | ADAMTS4 | NM_0050 | ADAMTS4 | AACATGGTGAAACCCTGTCTC | NM_022974_ | 4036 |
| | | 99 | mRNA | | idx3032 | |
| 813 | ADAMTS4 | NM_0050 | ADAMTS4 | ACAGGGTTTCACCATGTTGGC | NM_024022_ | 4041 |
| | | 99 | mRNA | | idx1935 | |
| 814 | ADAMTS4 | NM_0050 | ADAMTS4 | CCTGGCCAACATGGTGAAACC | ENSG | 4043 |
| | | 99 | mRNA | | 00000116032_ | |
| | | | | | idx5371 | |
| 815 | ADAMTS4 | NM_0050 | ADAMTS4 | GCCTGGCCAACATGGTGAAAC | ENSG | 4044 |
| | | 99 | mRNA | | 00000116032_ | |
| | | | | idx5370 | | |
| 816 | ADAMTS4 | NM_0050 | ADAMTS4 | AACTCCTGACCTCAGGTAATC | NM_005099_ | 4075 |
| | | 99 | mRNA | | idx4056 | |
| 817 | ADAMTS4 | NM_0050 | ADAMTS4 | TCACACCTGTAATCCCAGCAC | 5580991CA2_ | 4115 |
| | | 99 | mRNA | | idx142 | |
| 818 | ADAMTS4 | NM_0050 | ADAMTS4 | ACTCACACCTGTAATCCCAGC | NM_001226_ | 4117 |
| | | 99 | mRNA | | idx1024 | |
| 819 | ADAMTS4 | NM_0050 | ADAMTS4 | GCTCACACCTGTAATCCCAGC | 5580991CM_ | 4117 |
| | | 99 | mRNA | | idx140 | |
| 820 | ADAMTS4 | NM_0050 | ADAMTS4 | TAGAGCTGGAGCAGGACTC | NM_005099_ | 706 |
| | | 99 | mRNA | | idx685 | |
| 821 | ADAMTS4 | NM_0050 | ADAMTS4 | CTACCTGACTGGCACCATC | NM_005099_ | 803 |
| | | 99 | mRNA | | idx782 | |
| 822 | ADAMTS4 | NM_0050 | ADAMTS4 | ATCCTACGCCGGAAGAGTC | NM_005099_ | 963 |
| | | 99 | mRNA | | idx942 | |
| 823 | ADAMTS4 | NM_0050 | ADAMTS4 | GAGCCAAGCGCTTTGCTTC | NM_005099_ | 1051 |
| | | 99 | mRNA | | idx1030 | |
| 824 | ADAMTS4 | NM_0050 | ADAMTS4 | TGAGTAGATITGTGGAGAC | NM_005099_ | 1072 |
| | | 99 | mRNA | | idx1051 | |
| 825 | ADAMTS4 | NM_0050 | ADAMTS4 | ACTGGTGGTGGCAGATGAC | NM_005099_ | 1091 |
| | | 99 | mRNA | | idx1070 | |
| 826 | ADAMTS4 | NM_0050 | ADAMTS4 | CAGTGATGGCAGCAGCAGC | NM_005099_ | 1156 |
| | | 99 | mRNA | | idx1135 | |
| 827 | ADAMTS4 | NM_0050 | ADAMTS4 | GGCCTTCAAGCACCCAAGC | NM_005099_ | 1178 |
| | | 99 | mRNA | | idx1157 | |
| 828 | ADAMTS4 | NM_0050 | ADAMTS4 | CACTTTGACACAGCCATTC | NM_005099_ | 1350 |
| | | 99 | mRNA | | idx1329 | |
| 829 | ADAMTS4 | NM_0050 | ADAMTS4 | ACAGCCATTCTGTTTACCC | NM_005099_ | 1359 |
| | | 99 | mRNA | | idx1338 | |
| 830 | ADAMTS4 | NM_0050 | ADAMTS4 | CATGCTCCATGACAACTCC | NM_005099_ | 1529 |
| | | 99 | mRNA | | idx1508 | |
| 831 | ADAMTS4 | NM_0050 | ADAMTS4 | TGACTTCCTGGACAATGGC | NM_005099_ | 1664 |
| | | 99 | mRNA | | idx1643 | |
| 832 | ADAMTS4 | NM_0050 | ADAMTS4 | AATGGCTATGGGCACTGTC | NM_005099_ | 1677 |
| | | 99 | mRNA | | idx1656 | |
| 833 | ADAMTS4 | NM_0050 | ADAMTS4 | TGGCTATGGGCACTGTCTC | NM_005099_ | 1679 |
| | | 99 | mRNA | | idx1658 | |
| 834 | ADAMTS4 | NM_0050 | ADAMTS4 | AAACCAGAGGCTCCATTGC | NM_005099_ | 1704 |
| | | 99 | mRNA | | idx1683 | |
| 835 | ADAMTS4 | NM_0050 | ADAMTS4 | CCAGAGGCTCCATTGCATC | NM_005099_ | 1707 |
| | | 99 | mRNA | | idx1686 | |
| 836 | ADAMTS4 | NM_0050 | ADAMTS4 | GGACTATGATGCTGACCGC | NM_005099_ | 1748 |
| | | 99 | mRNA | | idx1727 | |
| 837 | ADAMTS4 | NM_0050 | ADAMTS4 | TCACGCCATTGTCCACAGC | NM_005099_ | 1794 |
| | | 99 | mRNA | | idx1773 | |
| 838 | ADAMTS4 | NM_0050 | ADAMTS4 | TATTCCACAGGCTGGTGGC | NM_005099_ | 1973 |
| | | 99 | mRNA | | idx1952 | |
| 839 | ADAMTS4 | NM_0050 | ADAMTS4 | CGACCTCTTCAAGAGCTTC | NM_005099_ | 2207 |
| | | 99 | mRNA | | idx2186 | |
| 840 | ADAMTS4 | NM_0050 | ADAMTS4 | CAGTGCAAACTCACCTGCC | NM_005099_ | 2277 |
| | | 99 | mRNA | | idx2256 | |
| 841 | ADAMTS4 | NM_0050 | ADAMTS4 | TACTATGTGCTGGAGCCAC | NM_005099_ | 2316 |
| | | 99 | mRNA | | idx2295 | |
| 842 | ADAMTS4 | NM_0050 | ADAMTS4 | GGTTCTGGTTGCAGCAAGC | NM_005099_ | 2469 |
| | | 99 | mRNA | | idx2448 | |
| 843 | ADAMTS4 | NM_0050 | ADAMTS4 | AACAATGTGGTCACTATCC | NM_005099_ | 2523 |
| | | 99 | mRNA | | idx2502 | |
| 844 | ADAMTS4 | NM_0050 | ADAMTS4 | TACACGCTGATGCCCTCCC | NM_005099_ | 2649 |
| | | 99 | mRNA | | idx2628 | |
| 845 | ADAMTS4 | NM_0050 | ADAMTS4 | GTCCTAGTGGCTGGCAACC | NM_005099_ | 2778 |
| | | 99 | mRNA | | idx2757 | |
| 846 | ADAMTS4 | NM_0050 | ADAMTS4 | ACGCCTCCGATACAGCTTC | NM_005099_ | 2807 |
| | | 99 | mRNA | | idx2786 | |
| 847 | ADAMTS4 | NM_0050 | ADAMTS4 | ATAACCTCACTATCCCGGC | NM_005099_ | 2936 |
| | | 99 | mRNA | | idx2915 | |
| 848 | ADAMTS4 | NM_0050 | ADAMTS4 | AGCCCTCCATCTAAACTGC | NM_005099_ | 3158 |
| | | 99 | mRNA | | idx3137 | |
| 849 | ADAMTS4 | NM_0050 | ADAMTS4 | AACCTGTTCTGCTTTCCTC | NM_005099_ | 3437 |
| | | 99 | mRNA | | idx3418 | |
| 850 | ADAMTS4 | NM_0050 | ADAMTS4 | CTGTTCTGCTTTCCTCTTC | NM_005099_ | 3440 |
| | | 99 | mRNA | | idx3421 | |
| 851 | ADAMTS4 | NM_0050 | ADAMTS4 | AGTCAAGGGTAGGGTGGGC | NM_005099_ | 3486 |
| | | 99 | mRNA | | idx3467 | |
| 852 | ADAMTS4 | NM_0050 | ADAMTS4 | AGAATCTCGCTCTGTCGCC | NM_005099_ | 3570 |
| | | 99 | mRNA | | idx3551 | |
| 853 | ADAMTS4 | NM_0050 | ADAMTS4 | TGGCACAATCTCGGCTCAG | NM_005099_ | 3604 |
| | | 99 | mRNA | | idx3585 | |
| 854 | ADAMTS4 | NM_0050 | ADAMTS4 | AATCTCGGCTCACTGCATC | NM_005099_ | 3610 |
| | | 99 | mRNA | | idx3591 | |
| 855 | ADAMTS4 | NM_0050 | ADAMTS4 | TCACTTGAACCCGGGAGGC | XM | 3633 |
| | | 99 | mRNA | | 050147_ | |
| | | | | | idx3544 | |
| 856 | ADAMTS4 | NM_0050 | ADAMTS4 | GTGATTCTCATGCCTCAGC | NM_005099_ | 3648 |
| | | 99 | mRNA | | idx3629 | |
| 857 | ADAMTS4 | NM_0050 | ADAMTS4 | TCCCAGCTACTCAGGAGGC | NM_013276_ | 3665 |
| | | 99 | mRNA | | idx3070 | |
| 858 | ADAMTS4 | NM_0050 | ADAMTS4 | TCCCAGCTACTCAGGAGGC | NM_014395_ | 3665 |
| | | 99 | mRNA | | idx2606 | |
| 859 | ADAMTS4 | NM_0050 | ADAMTS4 | AGTAGCTGGGATTACAGGC | NM_016225_ | 3673 |
| | | 99 | mRNA | | idx1419 | |
| 860 | ADAMTS4 | NM_0050 | ADAMTS4 | AGAGTCTCGCTATTGTCAC | NM_005099_ | 3739 |
| | | 99 | mRNA | | idx3720 | |
| 861 | ADAMTS4 | NM_0050 | ADAMTS4 | CTGGGTTCCAGCAATTCTC | NM_005099_ | 3798 |
| | | 99 | mRNA | | idx3779 | |
| 862 | ADAMTS4 | NM_0050 | ADAMTS4 | GCAATTCTCCTGCCTCAGC | NM_007181_ | 3808 |
| | | 99 | mRNA | | idx2505 | |
| 863 | ADAMTS4 | NM_0050 | ADAMTS4 | CTCCTGACCTTAGGTGATC | NM_005099_ | 3930 |
| | | 99 | mRNA | | idx3911 | |
| 864 | ADAMTS4 | NM_0050 | ADAMTS4 | TCCTGACCTTAGGTGATCC | NM_005099_ | 3931 |
| | | 99 | mRNA | | idx3912 | |
| 865 | ADAMTS4 | NM_0050 | ADAMTS4 | ACGCCTGTAATCCCAGCAC | ENSG | 3970 |
| | | 99 | mRNA | | 00000116032_ | |
| | | | | | idx3384 | |
| 866 | ADAMTS4 | NM_0050 | ADAMTS4 | TGGGATTACAGGCGTGAGC | NM_024628_ | 3974 |
| | | 99 | mRNA | | idx2003 | |
| 867 | ADAMTS4 | NM_0050 | ADAMTS4 | GGTGAAACCCTGTCTCTAC | ENSG | 4033 |
| | | 99 | mRNA | | 100000115257_ | |
| | | | | | idx1012 | |
| 868 | ADAMTS4 | NM_0050 | ADAMTS4 | CATGGTGAAACCCTGTCTC | NM_022973_ | 4036 |
| | | 99 | mRNA | | idx3029 | |
| 869 | ADAMTS4 | NM_0050 | ADAMTS4 | CATGGTGAAACCCTGTCTC | NM_022974_ | 4036 |
| | | 99 | mRNA | | idx3032 | |
| 870 | ADAMTS4 | NM_0050 | ADAMTS4 | AGGGTTTCACCATGTTGGC | NM_024022_ | 4041 |
| | | 99 | mRNA | | idx1938 | |
| 871 | ADAMTS4 | NM_0050 | ADAMTS4 | TGGCCAACATGGTGAAACC | ENSG | 4043 |
| | | 99 | mRNA | | 00000116032_ | |
| | | | | | idx5371 | |
| 872 | ADAMTS4 | NM_0050 | ADAMTS4 | CTGGCCAACATGGTGAAAC | ENSG | 4044 |
| | | 99 | mRNA | | 00000116032_ | |
| | | | | | idx5370 | |
| 873 | ADAMTS4 | NM_0050 | ADAMTS4 | CTCCTGACCTCAGGTAATC | NM_005099_ | 4075 |
| | | 99 | mRNA | | idx4056 | |
| 874 | ADAMTS4 | NM_0050 | ADAMTS4 | ACACCTGTAATCCCAGCAC | 5580991CA2_ | 4115 |
| | | 99 | mRNA | | idx142 | |
| 875 | ADAMTS4 | NM_0050 | ADAMTS4 | TCACACCTGTAATCCCAGC | NM_001226_ | 4117 |
| | | 99 | mRNA | | idx1024 | |
| 876 | ADAMTS4 | NM_0050 | ADAMTS4 | GCTCACACCTGTAATCCCA | 5580991CA2_ | 4117 |
| | | 99 | mRNA | | idx140 | |

The loop sequence, 5′ UUGCUAUA-3′ (SEQ ID NO: 13) is used to make a self-complementing siRNA.

Adenoviral knock down constructs are used to transduce mouse, rat or human primary neuronal cells and/or cell lines (e.g. HEK293, SH-SY5Y, IMR-32, SK-N-SH, SK-N-MC, H4, CHO, COS, HeLa) stably over-expressing APPwt or not . 24 h later, the adenoviruses are removed and fresh medium is added to the cells. 96 h later, the medium of the cells is refreshed to allow the accumulation of amyloid beta 1-42 peptides. After 48 h, the conditioned medium of these cells is assayed using the amyloid beta 1-42 ELISA, which is performed as described in EXAMPLE 1. Co-infection of SH-SY5Y cells with adenoviruses expressing APPwt and a USP21, GZMM, USP2, or ADAMTS4 KD sequence reduces amyloid beta 1-42 levels in the conditioned medium compared to GL2 KD virus infected cells. In addition, RNA is isolated from these infected cells and USP21, GZMM, USP2, and ADAMTS4 RNA levels are determined via real time PCR. Determination of the levels of household keeping genes allows the normalization of RNA levels of the target gene between different RNA samples, represented as delta Ct values. USP21, GZMM, USP2, and ADAMTS4 RNA levels are reduced in cells infected with the USP21, GZMM, USP2, and ADAMTS4 adenoviral KD virus; accordingly, USP21, GZMM, USP2, and ADAMTS4 are effective for the reduction of secreted amyloid beta peptide 1-42 levels.

### Example 6

### Identification of Small Molecules that Inhibit Protease Activity

Compounds are screened for inhibition of the activity of the polypeptides of the present invention. The affinity of the compounds to the polypeptides is determined in an experiment detecting changes in levels of cleaved substrate. In brief, the polypeptides of the present invention are incubated with its substrate in an appropriate buffer. The combination of these components results in the cleavage of the substrate.

The polypeptides can be applied as complete polypeptides or as polypeptide fragments, which still comprise the catalytic activity of the polypeptide of the invention.

Cleavage of the substrate can be followed in several ways. In a first method, the substrate protein is heavily labeled with a fluorescent dye, like fluorescein, resulting in a complete quenching of the fluorescent signal. Cleavage of the substrate however, releases individual fragments, which contain less fluorescent labels. This results in the loss of quenching and the generation of a fluorescent signal, which correlates to the levels of cleaved substrate. Cleavage of the protein, which results in smaller peptide fragments, can also be measured using fluorescent polarization (FP). Alternatively, cleavage of the substrate can also be detected using fluorescence resonance energy transfer (FRET): a peptide substrate is labeled on both sides with either a quencher and fluorescent molecule, like DABCYL and EDANS. Upon cleavage of the substrate both molecules are separated resulting in fluorescent signal correlating to the levels of cleaved substrate. In addition, cleavage of a peptide substrate can also generate a new substrate for another enzymatic reaction, which is then detected via a fluorescent, chemiluminescent or colorimetric method.

Small molecules are randomly screened or are preselected based upon drug class, i.e. protease, or upon virtual ligand screening (VLS) results. VLS uses virtual docking technology to test large numbers of small molecules in silico for their binding to the polypeptide of the invention. Small molecules are added to the proteolytic reaction and their effect on levels of cleaved substrate is measured with the described technologies.

Small molecules that inhibit the protease activity are identified and are subsequently tested at different concentrations. IC50 values are calculated from these dose response curves. Strong binders have an IC50 in the nanomolar and even picomolar range. Compounds that have an IC50 of at least 10 micromol or better (nmol to pmol) are applied in amyloid beta secretion assay to check for their effect on the beta amyloid secretion and processing.

1. A method for identifying a compound that inhibits the processing of amyloid-beta precursor protein in a mammalian cell, comprising
(a) contacting a compound with a polypeptide comprising an amino acid sequence selected from the group consisting of SEQ ID NO: 7, 8, 9, and 10; and
(b) measuring a compound-polypeptide property related to the production of amyloid-beta peptide.

2. The method according to claim 1, wherein said polypeptide is in an in vitro cell-free preparation.

3. The method according to claim 2, wherein said polypeptide is present in a mammalian cell.

4. The method of claim 1, wherein said property is a binding affinity of said compound to said polypeptide.

5. The method of claim 3, wherein said property is activation of a biological pathway producing an indicator of the processing of amyloid-beta precursor protein.

6. The method of claim 5 wherein said indicator is amyloid-beta peptide.

7. The method of claim 6 wherein said amyloid-beta peptide is selected from the group consisting of one or more of amyloid-beta peptide 1-42, 1-40, 11-42 and 11-40.

8. The method of claim 7 wherein said amyloid-beta peptide is amyloid-beta peptide 1-42.

9. The method according to claim 2, wherein said compound is a peptide in a phage display library or an antibody fragment library.

10. The method according to claim 1, wherein said compound is an aggrecanase inhibitor.

11. An agent for the inhibition of amyloid-beta precursor processing selected from the group consisting of an antisense polynucleotide, a ribozyme, and a small interfering RNA (siRNA), wherein said agent comprises a nucleic acid sequence complementary to, or engineered from, a naturally-occurring polynucleotide sequence encoding a polypeptide comprising an amino acid sequence selected from the group consisting of SEQ ID NO: 7, 8, 9, and 10.

12. The agent according to claim 11, wherein a vector in a mammalian cell expresses said agent.

13. The agent according to claim 12, wherein said vector is an adenoviral, retroviral, adeno-associated viral, lentiviral, a herpes simplex viral or a sendaiviral vector.

14. The agent according to claim 13, wherein said antisense polynucleotide and said siRNA comprise an antisense strand of 17-25 nucleotides complementary to a sense strand, wherein said sense strand is selected from 17-25 continuous nucleotides of a nucleic acid sequence selected from the group consisting of SEQ ID NO: 14-32, 49-68, and 75-619.

15. The agent according to claim 14, wherein said siRNA further comprises said sense strand.

16. The agent according to claim 15, wherein said sense strand is selected from 17-25 continuous nucleotides of a nucleic acid sequence selected from the group consisting of SEQ ID NO: 1, 2, 3, and 4.

17. The agent according to claim 16, wherein said siRNA further comprises a loop region connecting said sense and said antisense strand.

18. The agent according to claim 17 wherein said loop region comprises a nucleic acid sequence defined of SEQ ID NO: 13.

19. The agent according to claim 11, wherein said agent is an antisense polynucleotide, ribozyme, or siRNA comprising a nucleic acid sequence selected from the group consisting of SEQ ID NO: 14-32, 49-68, and 75-619.

20. A cognitive enhancing pharmaceutical composition comprising a therapeutically effective amount of an agent of claim 11 in admixture with a pharmaceutically acceptable carrier.

21. The cognitive enhancing pharmaceutical composition according to claim 20 wherein said agent comprises a polynucleotide comprising a nucleic acid sequence selected from the group consisting of SEQ ID NO: 14-32, 49-68, and 75-619, a polynucleotide complementary to said nucleic acid sequence, and a combination thereof.

22. A method of inhibiting the processing of amyloid-beta precursor protein in a subject suffering or susceptible to the abnormal processing of said protein, comprising administering to said subject a pharmaceutical composition according to claim 21.

23. A method according to claim 22 for treatment or prevention of a condition involving cognitive impairment or a susceptibility to the condition.

24. The method according to claim 23 wherein the condition is Alzheimer's disease.

25. A pharmaceutical composition for the treatment or prevention of a condition involving cognitive impairment or a susceptibility to the condition, comprising an effective amyloid-beta precursor processing-inhibiting amount of a mitogen activated protein-protease inhibitor.

26. A composition according to claim 25, wherein said mitogen activated protein-protease inhibitor is selected from the group consisting of N1-(2(R)-hydroxy-1(S)-indanyl)-N4-hydroxy-2(R)-substituted-butanediamides, and pharmaceutically acceptable salts, hydrates, solvates, or prodrugs thereof in admixture with a pharmaceutically acceptable carrier.

27. A pharmaceutical composition according to claim 20, further comprising labeling indicating use of said composition for the treatment or prevention of a condition involving cognitive impairment or a susceptibility to said condition.

28. A pharmaceutical composition according to claim 25, further comprising labeling indicating use of said composition for the treatment or prevention of a condition involving cognitive impairment or a susceptibility to said condition.
